(19) United States
(12) Patent Application Publication
(10) Pub. No.: US 2019/0378994 A1
(43) Pub. Date: Dec. 12, 2019

(54) ORGANIC COMPOUND AND ORGANIC ELECTROLUMINESCENCE DEVICE USING THE SAME

(71) Applicant: LUMINESCENCE TECHNOLOGY CORPORATION, Hsinchu (TW)

(72) Inventors: FENG-WEN YEN, Taipei (TW); LI-CHIEH CHUANG, Nantou (TW); SHU-HUA YEH, Hsinchu (TW)

(73) Assignee: LUMINESCENCE TECHNOLOGY CORPORATION, Hsinchu (TW)

(21) Appl. No.: 16/005,677

(22) Filed: Jun. 12, 2018

(51) Int. Cl. H01L 51/00; C07D 491/048; C07D 487/04; C07D 495/04; C07D 333/76; C07D 307/91; C07D 209/86; C07D 409/04; C07D 409/10; C07D 403/10; C07D 405/04; H01L 51/50

(52) U.S. Cl. CPC H01L 51/0072; H01L 51/5206; C07D 487/04; C07D 495/04; C07D 333/76; C07D 307/91; C07D 209/86; C07D 409/04; C07D 409/10; C07D 403/10; C07D 405/04; H01L 51/5076; H01L 51/5024; H01L 51/5016; H01L 51/0074; H01L 51/0073; C07D 491/048

(57) ABSTRACT

An organic compound which can be used as the phosphorescent host material, the fluorescent host material, or the fluorescent dopant material of the light emitting layer, and/or the electron transporting material of the organic electroluminescence device is disclosed. The organic electroluminescence device employing the organic compound can lower driving voltage, prolong half-lifetime, and increase luminance and current efficiency.

### FIELD OF INVENTION

The present invention relates to a novel organic compound and, more particularly, to an organic electroluminescence device using the organic compound.

### BACKGROUND OF THE INVENTION

An organic electroluminescence (organic EL) device is an organic light-emitting diode (OLED) in which the light emitting layer is a film made from organic compounds, which emits light in response to the electric current. The light emitting layer containing the organic compound is sandwiched between two electrodes. The organic EL device is applied to flat panel displays due to its high illumination, low weight, ultra-thin profile, self-illumination without back light, low power consumption, wide viewing angle, high contrast, simple fabrication methods and rapid response time.

Typically, the organic EL device is composed of organic material layers sandwiched between two electrodes. The organic material layers include, e.g., hole injection layer (HIL), hole transporting layer (HTL), emitting layer (EML), electron transporting layer (ETL), and electron injection layer (EIL). The basic mechanism of organic EL involves the injection, transport, and recombination of carriers as well as exciton formation for emitting light. When an external voltage is applied across the organic EL device, electrons and holes are injected from the cathode and the anode, respectively. Electrons will be injected from the cathode into a LUMO (lowest unoccupied molecular orbital) and holes will be injected from the anode into a HOMO (highest occupied molecular orbital). Subsequently, the electrons recombine with holes in the light emitting layer to form excitons, which then deactivate to emit light. When luminescent molecules absorb energy to achieve an excited state, the exciton may either be in a singlet state or a triplet state, depending on how the spins of the electrons and holes have been combined. It is well known that the excitons formed under electrical excitation typically include 25% singlet excitons and 75% triplet excitons. In the fluorescence materials, however, the electrically generated energy in the 75% triplet excitons will be dissipated as heat for decay from the triplet state is spin forbidden. Therefore, a fluorescent electroluminescence device has only 25% internal quantum efficiency, which leads to the theoretically highest external quantum efficiency (EQE) of only 5% due to only ˜20% of the light out-coupling efficiency of the device. In contrast to fluorescent electroluminescence devices, phosphorescent organic EL devices make use of spin-orbit interactions to facilitate intersystem crossing between singlet and triplet states, thus obtaining emission from both singlet and triplet states and the internal quantum efficiency of electroluminescence devices from 25% to 100%.

For full-colored flat panel displays using organic EL devices, the organic materials used in the organic EL devices are still unsatisfactory in half-life time, power consumption, luminance, and current efficiency. Therefore, there is still a need for an organic compound that can lower the driving voltage, increase the current efficiency and luminance, and prolong the half-life time for the organic EL device.

### SUMMARY OF THE INVENTION

Accordingly, an object of the invention is to provide a novel organic compound and an organic EL device using the same, which can exhibit improved luminance, current efficiency, and half-life time.

Another object of the invention is to provide a novel organic compound and an organic EL device using the same, which can operate under reduced voltage and exhibit higher current efficiency and longer half-life time.

Still another object of the present invention is to provide an organic compound, which can be used as a phosphorescent host material, a fluorescent host material, or a fluorescent dopant material in the emitting layer, and/or an electron transporting material in an organic EL device to improve the power consumption, luminance, current efficiency, or life time.

According to the present invention, an organic compound which can be used in organic EL devices is disclosed. The organic compound is represented by the following formula (1) or formula (2):

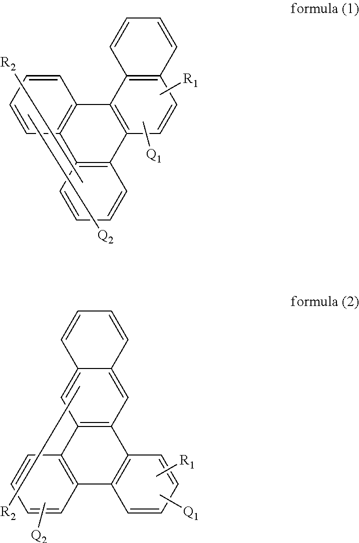

wherein one of Q₁ and Q₂ represents formula (3) below:

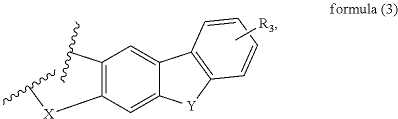

wherein X and Y are independently a divalent bridge selected from the group consisting of O, S, Se, NR₄, CR₅R₆, and SiR₇R₈; R₁ to R₃ are independently absent, a hydrogen atom, a halide, a substituted or unsubstituted aryl group having 5 to 50 ring atoms, a substituted or unsubstituted heteroaryl group having 5 to 50 ring atoms, a substituted or unsubstituted arylamine group having 5 to 50 ring atoms, or a substituted or unsubstituted heteroarylamine group having 5 to 50 ring atoms; R₄ is a hydrogen atom, a halide, a substituted or unsubstituted aryl group having 5 to 50 ring atoms, a substituted or unsubstituted heteroaryl group having 5 to 50 ring atoms, a substituted or unsubstituted arylamine group having 5 to 50 ring atoms, or a substituted or unsubstituted heteroarylamine group having 5 to 50 ring atoms; and R₅ to R₈ are independently a hydrogen atom, a halide, a substituted or unsubstituted alkyl group having 1 to 30 carbon atoms, a substituted or unsubstituted aryl group having 6 to 30 carbon atoms, a substituted or unsubstituted aralkyl group having 6 to 30 carbon atoms, or a substituted or unsubstituted heteroaryl group having 3 to 30 carbon atoms.

The present invention further discloses an organic electroluminescence device. The organic electroluminescence device comprises a pair of electrodes composed of a cathode and an anode, and a light emitting layer and one or more organic thin film layers between the pair of electrodes. At least one of the light emitting layer and the organic thin film layer comprises the organic compound of formula (1) or formula (2).

### BRIEF DESCRIPTION OF THE DRAWINGS

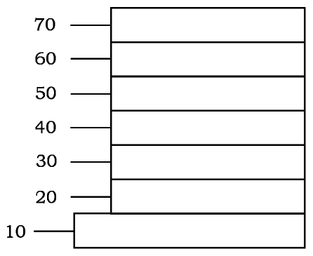

The FIG. 1s a schematic view showing an organic EL device according to an embodiment of the present invention.

### DESCRIPTION OF THE PREFERRED EMBODIMENTS

What probed into the invention is the organic compound and organic EL device using the organic compound. Detailed descriptions of the production, structure and elements will be provided as follows such that the invention can be fully understood. Obviously, the application of the invention is not confined to specific details familiar to those skilled in the art. On the other hand, the common elements and procedures that are known to everyone are not described in details to avoid unnecessary limits of the invention. Some preferred embodiments of the present invention will now be described in greater detail as follows. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

In one embodiment of the present invention, an organic compound which can be used as the phosphorescent host material, the fluorescent host material, or the fluorescent dopant material of the light emitting layer, and/or the electron transporting material of the organic EL device is disclosed. The organic compound is represented by the following formula (1) or formula (2):

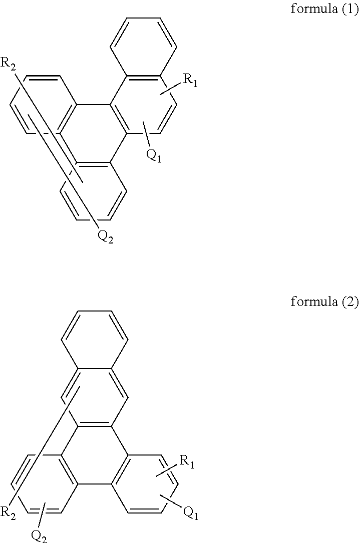

wherein one of Q₁ and Q₂ represents formula (3) below:

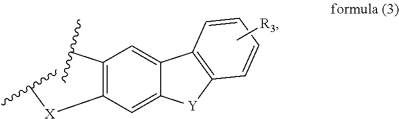

wherein X and Y are independently a divalent bridge selected from the group consisting of O, S, Se, NR₄, CR₅R₆, and SiR₇R₈; R₁ to R₃ are independently absent, a hydrogen atom, a halide, a substituted or unsubstituted aryl group having 5 to 50 ring atoms, a substituted or unsubstituted heteroaryl group having 5 to 50 ring atoms, a substituted or unsubstituted arylamine group having 5 to 50 ring atoms, or a substituted or unsubstituted heteroarylamine group having 5 to 50 ring atoms; R₄ is a hydrogen atom, a halide, a substituted or unsubstituted aryl group having 5 to 50 ring atoms, a substituted or unsubstituted heteroaryl group having 5 to 50 ring atoms, a substituted or unsubstituted arylamine group having 5 to 50 ring atoms, or a substituted or unsubstituted heteroarylamine group having 5 to 50 ring atoms; and R₅ to R₈ are independently a hydrogen atom, a halide, a substituted or unsubstituted alkyl group having 1 to 30 carbon atoms, a substituted or unsubstituted aryl group having 6 to 30 carbon atoms, a substituted or unsubstituted aralkyl group having 6 to 30 carbon atoms, or a substituted or unsubstituted heteroaryl group having 3 to 30 carbon atoms.

In some embodiments, the organic compound is represented by one of the following formula (4) to formula (11):

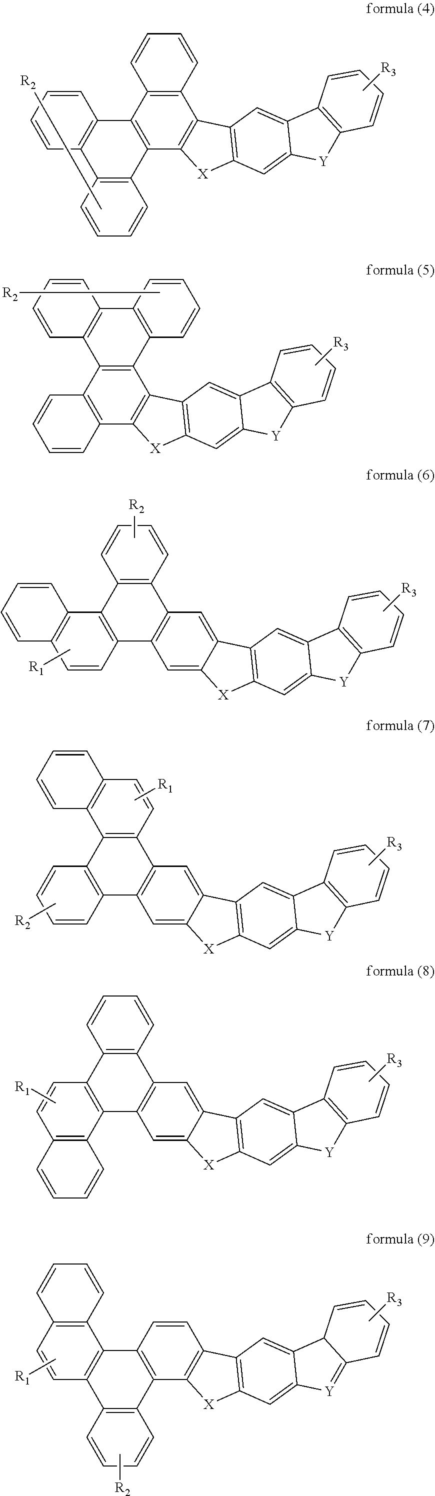

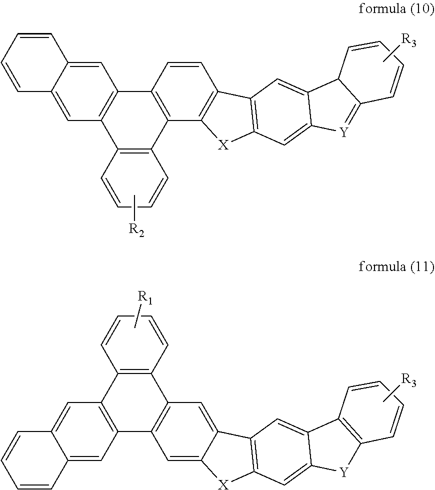

wherein X and Y are independently a divalent bridge selected from the group consisting of O, S, Se, NR₄, CR₅R₆, and SiR₇R₈; R₁ to R₃ are independently absent, a hydrogen atom, a halide, a substituted or unsubstituted aryl group having 5 to 50 ring atoms, a substituted or unsubstituted heteroaryl group having 5 to 50 ring atoms, a substituted or unsubstituted arylamine group having 5 to 50 ring atoms, or a substituted or unsubstituted heteroarylamine group having 5 to 50 ring atoms; R₄ is a hydrogen atom, a halide, a substituted or unsubstituted aryl group having 5 to 50 ring atoms, a substituted or unsubstituted heteroaryl group having 5 to 50 ring atoms, a substituted or unsubstituted arylamine group having 5 to 50 ring atoms, or a substituted or unsubstituted heteroarylamine group having 5 to 50 ring atoms; and R₅ to R₈ are independently a hydrogen atom, a halide, a substituted or unsubstituted alkyl group having 1 to 30 carbon atoms, a substituted or unsubstituted aryl group having 6 to 30 carbon atoms, a substituted or unsubstituted aralkyl group having 6 to 30 carbon atoms, or a substituted or unsubstituted heteroaryl group having 3 to 30 carbon atoms.

In some embodiments, R₁ to R₄ are independently a substituted or unsubstituted phenyl group, a substituted or unsubstituted biphenyl group, a substituted or unsubstituted fluorene group, a substituted or unsubstituted benzofluorene group, a substituted or unsubstituted naphthyl group, a substituted or unsubstituted anthracenyl group, a substituted or unsubstituted phenanthrenyl group, a substituted or unsubstituted pyrenyl group, a substituted or unsubstituted chrysenyl group, a substituted or unsubstituted triphenylenyl group, a substituted or unsubstituted benzimidazole group, a substituted or unsubstituted carbazolyl group, a substituted or unsubstituted biscarbazolyl group, a substituted or unsubstituted dibenzofuranyl group, a substituted or unsubstituted dibenzothiophenyl group, a substituted or unsubstituted diphenylphosphine oxide group, a substituted or unsubstituted triazinyl group, a substituted or unsubstituted diazinyl group, a substituted or unsubstituted pyridinyl group, a substituted or unsubstituted phenanthroline group, a substituted or unsubstituted dihydroacridine group, a substituted or unsubstituted phenothiazine group, a substituted or unsubstituted phenoxazine group, a substituted or unsubstituted dihydrophenazine group, a substituted or unsubstituted diphenylamine group, a substituted or unsubstituted triphenylamine group, a substituted or unsubstituted phenyldibenzofuranylamine group, or a substituted or unsubstituted phenyldibenzothiophenylamine group.

In some embodiments, R₁ to R₄ independently represent one of the following substituents:

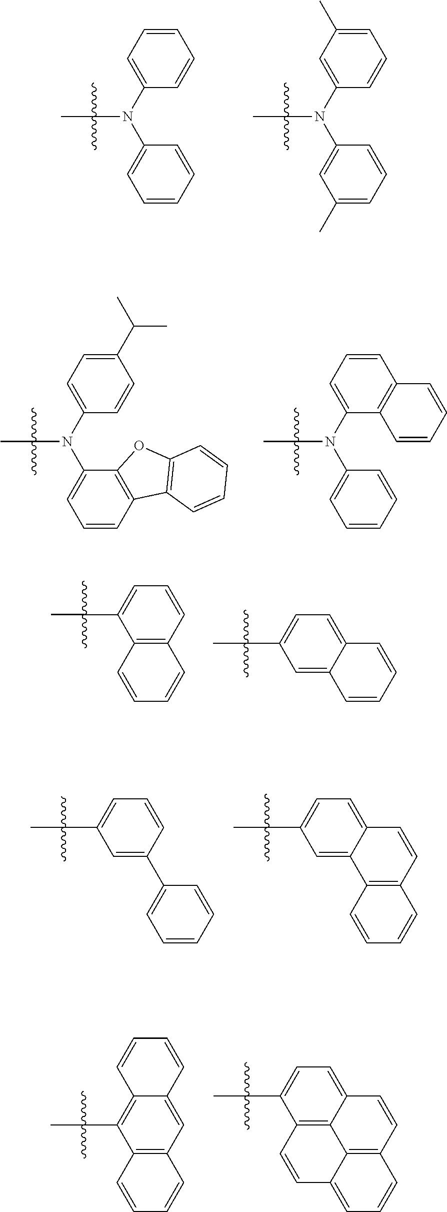

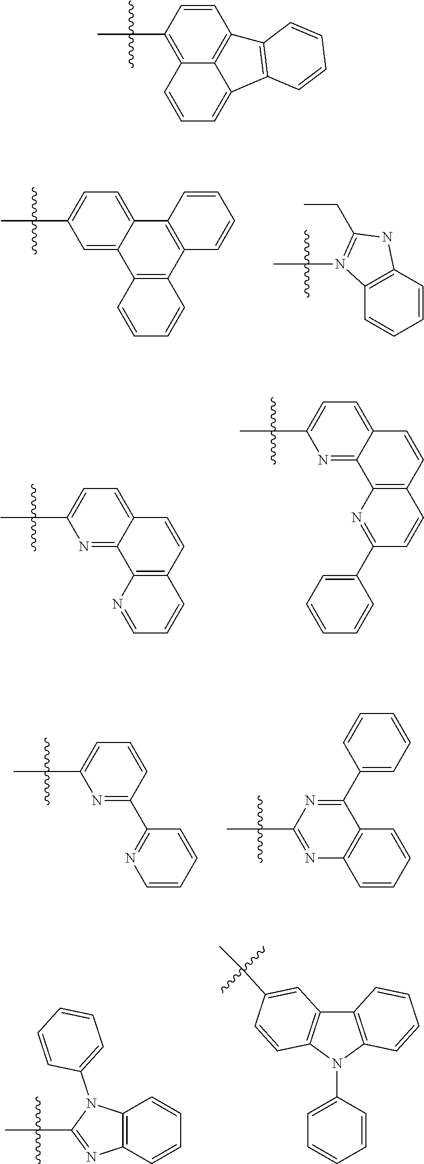

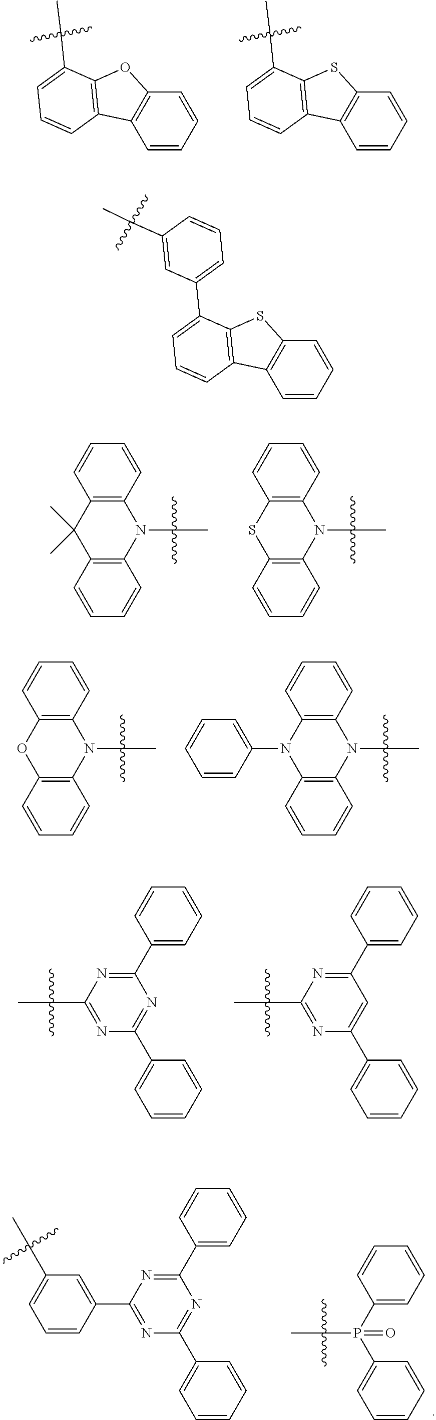

Preferably, the organic compound is one of the following compounds:

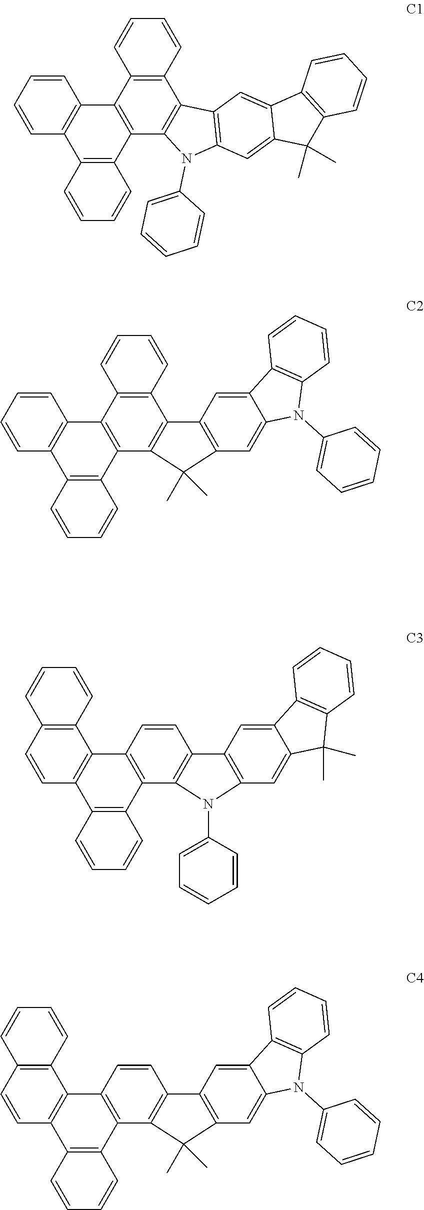 [SMILES: CC1(C)c2ccccc2-c2cc3c4c5ccccc5c5c6ccccc6c6ccccc6c5c4n(-c4ccccc4)c3cc21 ; CC1(C)c2cc3c(cc2-c2c1c1c4ccccc4c4ccccc4c1c1ccccc21)c1ccccc1n3-c1ccccc1 ; CC1(C)c2ccccc2-c2cc3c4ccc5c6c7ccccc7ccc6c6ccccc6c5c4n(-c4ccccc4)c3cc21 ; CC1(C)c2cc3c(cc2-c2ccc4c(c21)c1ccccc1c1ccc2ccccc2c14)c1ccccc1n3-c1ccccc1]

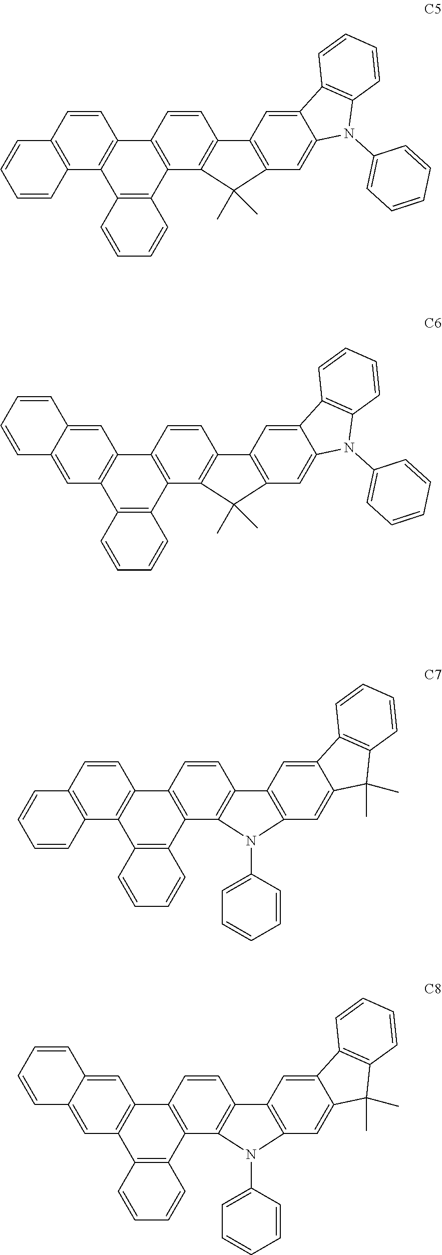 [SMILES: CC1(C)c2cc3c(cc2-c2ccc4c5ccc6ccccc6c5c5ccccc5c4c21)c1ccccc1n3-c1ccccc1 ; CC1(C)c2cc3c(cc2-c2ccc4c5cc6ccccc6cc5c5ccccc5c4c21)c1ccccc1n3-c1ccccc1 ; CC1(C)c2ccccc2-c2cc3c4ccc5c6ccc7ccccc7c6c6ccccc6c5c4n(-c4ccccc4)c3cc21 ; CC1(C)c2ccccc2-c2cc3c4ccc5c6cc7ccccc7cc6c6ccccc6c5c4n(-c4ccccc4)c3cc21]

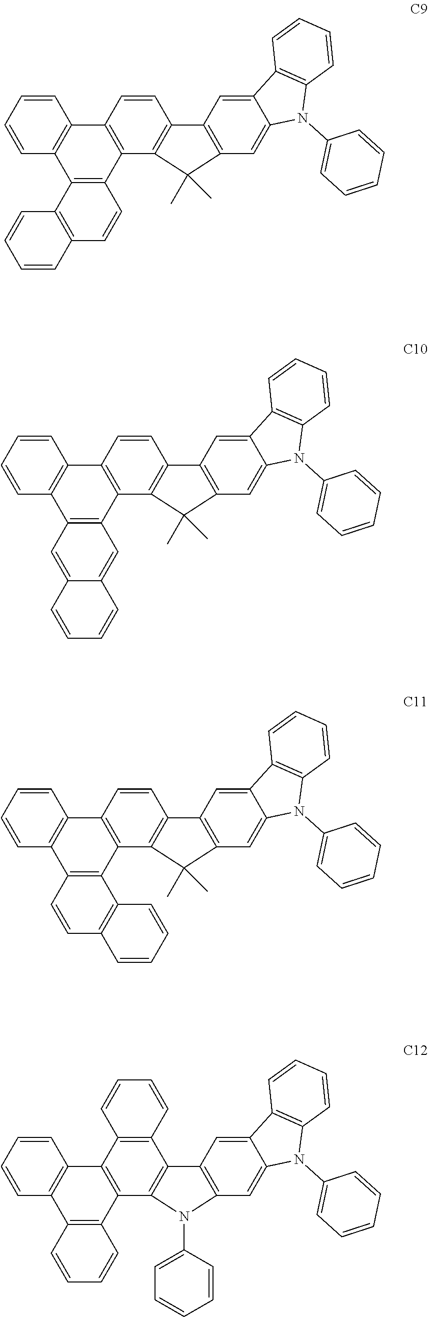 [SMILES: CC1(C)c2cc3c(cc2-c2ccc4c5ccccc5c5c6ccccc6ccc5c4c21)c1ccccc1n3-c1ccccc1 ; CC1(C)c2cc3c(cc2-c2ccc4c5ccccc5c5cc6ccccc6cc5c4c21)c1ccccc1n3-c1ccccc1 ; CC1(C)c2cc3c(cc2-c2ccc4c5ccccc5c5ccc6ccccc6c5c4c21)c1ccccc1n3-c1ccccc1 ; c1ccc(-n2c3ccccc3c3cc4c5c6ccccc6c6c7ccccc7c7ccccc7c6c5n(-c5ccccc5)c4cc32)cc1]

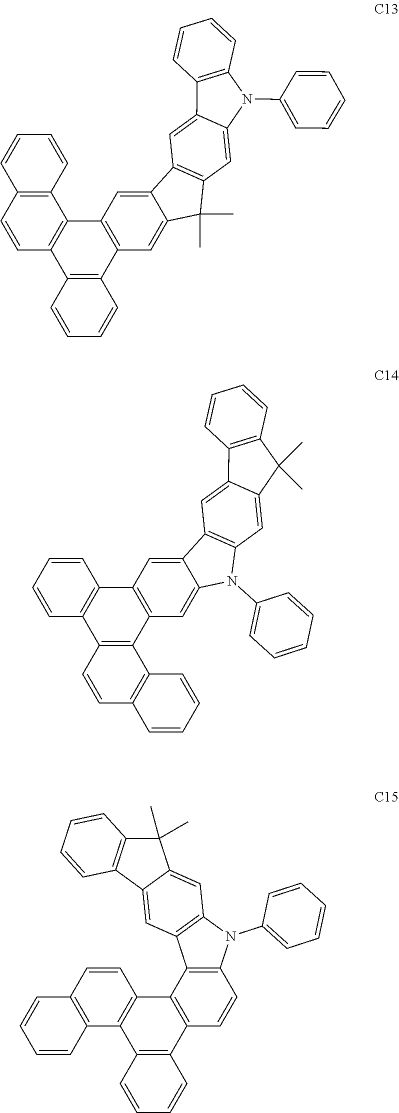 [SMILES: CC1(C)c2cc3c4ccccc4c4ccc5ccccc5c4c3cc2-c2cc3c4ccccc4n(-c4ccccc4)c3cc21 ; CC1(C)c2ccccc2-c2cc3c4cc5c6ccccc6c6ccc7ccccc7c6c5cc4n(-c4ccccc4)c3cc21 ; CC1(C)c2ccccc2-c2cc3c4c5c(ccc4n(-c4ccccc4)c3cc21)c1ccccc1c1c2ccccc2ccc15]

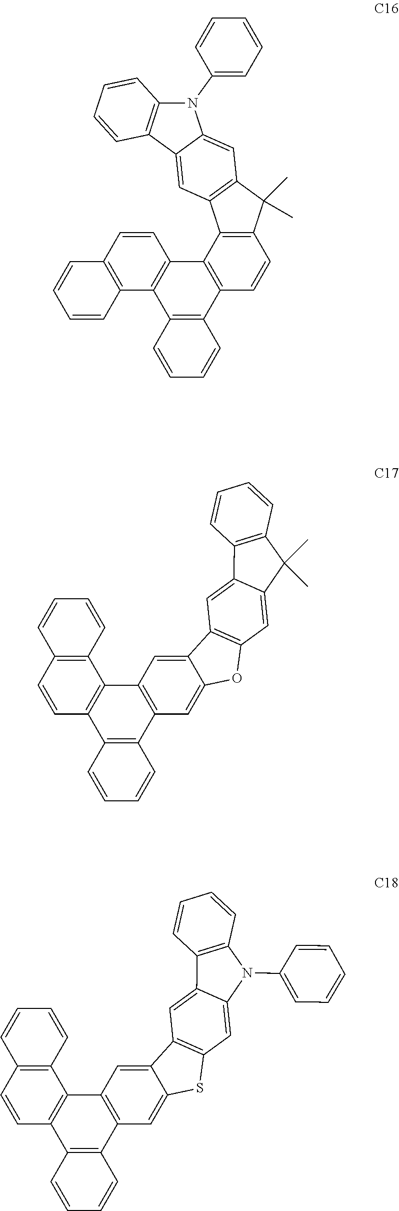 [SMILES: CC1(C)c2cc3c(cc2-c2c1ccc1c4ccccc4c4c5ccccc5ccc4c21)c1ccccc1n3-c1ccccc1 ; CC1(C)c2ccccc2-c2cc3c(cc21)oc1cc2c4ccccc4c4ccc5ccccc5c4c2cc13 ; c1ccc(-n2c3ccccc3c3cc4c(cc32)sc2cc3c5ccccc5c5ccc6ccccc6c5c3cc24)cc1]

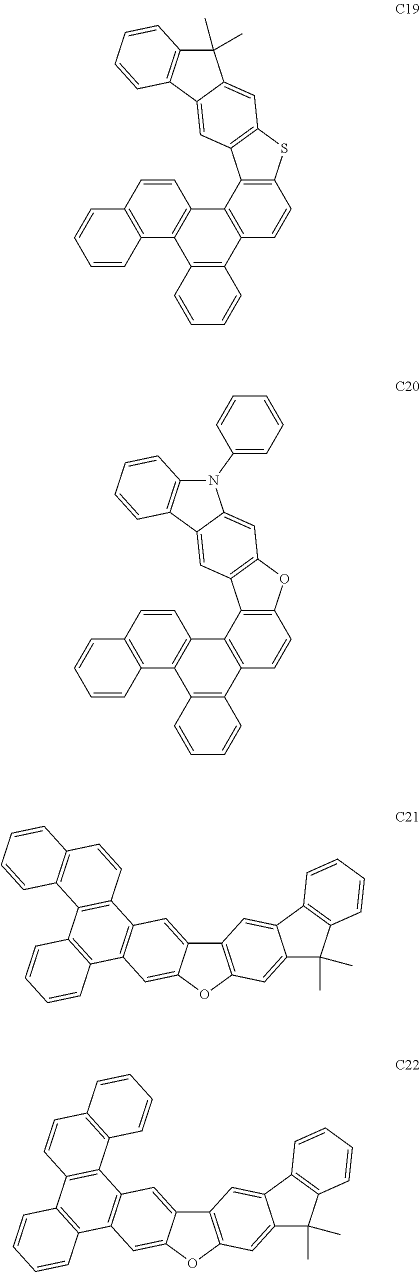 [SMILES: CC1(C)c2ccccc2-c2cc3c(cc21)sc1ccc2c4ccccc4c4c5ccccc5ccc4c2c13 ; c1ccc(-n2c3ccccc3c3cc4c(cc32)oc2ccc3c5ccccc5c5c6ccccc6ccc5c3c24)cc1 ; CC1(C)c2ccccc2-c2cc3c(cc21)oc1cc2c4ccccc4c4c5ccccc5ccc4c2cc13 ; CC1(C)c2ccccc2-c2cc3c(cc21)oc1cc2c4ccccc4c4ccc5ccccc5c4c2cc13]

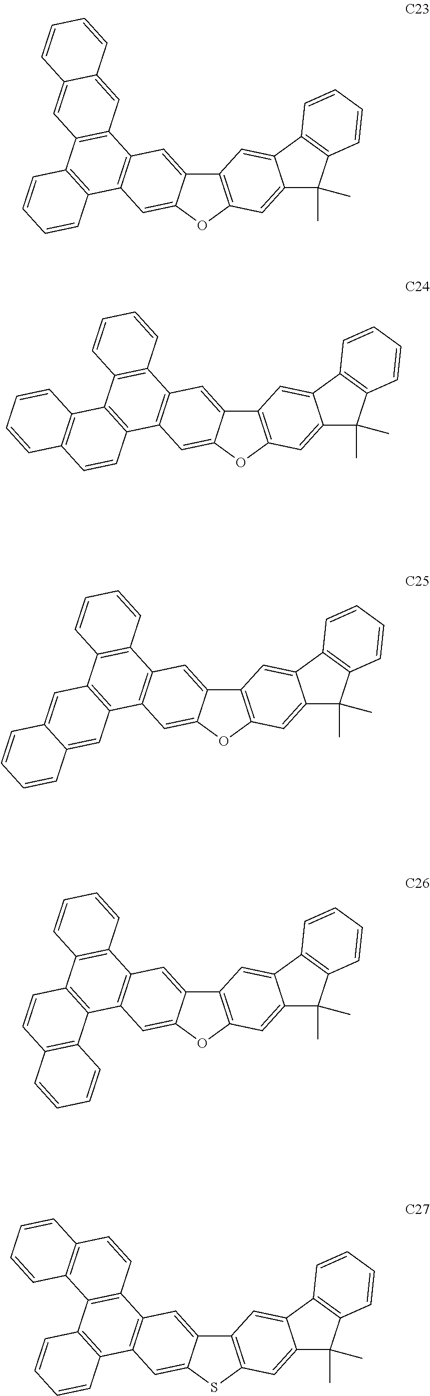 [SMILES: CC1(C)c2ccccc2-c2cc3c(cc21)oc1cc2c4ccccc4c4cc5ccccc5cc4c2cc13 ; CC1(C)c2ccccc2-c2cc3c(cc21)oc1cc2c(cc13)c1ccccc1c1c3ccccc3ccc21 ; CC1(C)c2ccccc2-c2cc3c(cc21)oc1cc2c4cc5ccccc5cc4c4ccccc4c2cc13 ; CC1(C)c2ccccc2-c2cc3c(cc21)oc1cc2c(cc13)c1ccccc1c1ccc3ccccc3c12 ; CC1(C)c2ccccc2-c2cc3c(cc21)sc1cc2c4ccccc4c4c5ccccc5ccc4c2cc13]

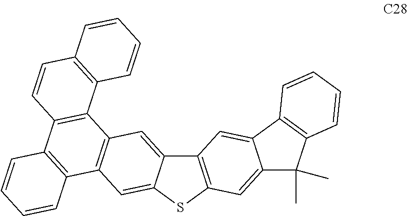 [SMILES: CC1(C)c2ccccc2-c2cc3c(cc21)sc1cc2c4ccccc4c4ccc5ccccc5c4c2cc13]

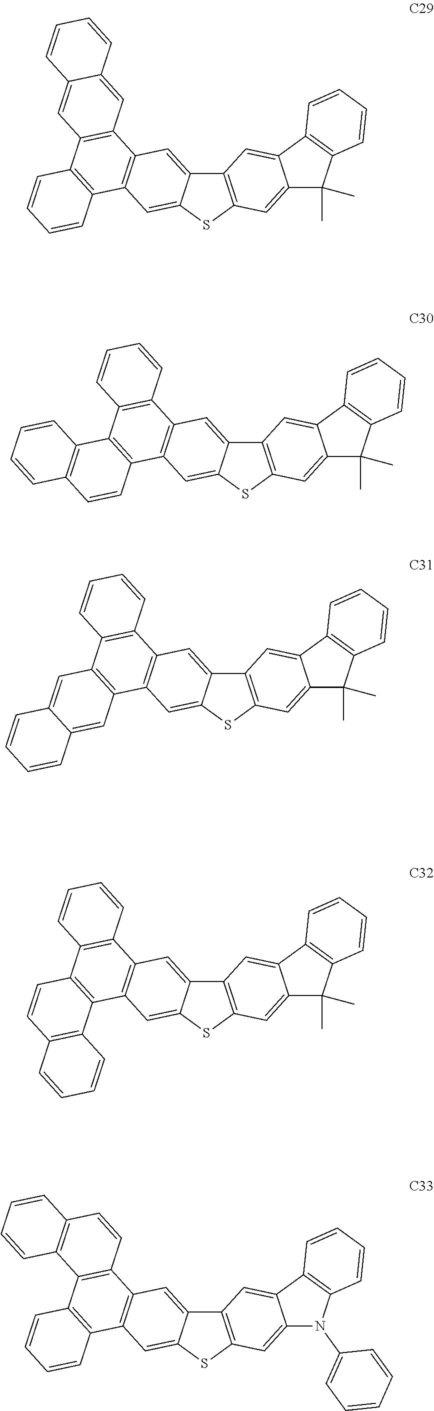 [SMILES: CC1(C)c2ccccc2-c2cc3c(cc21)sc1cc2c4ccccc4c4cc5ccccc5cc4c2cc13 ; CC1(C)c2ccccc2-c2cc3c(cc21)sc1cc2c(cc13)c1ccccc1c1c3ccccc3ccc21 ; CC1(C)c2ccccc2-c2cc3c(cc21)sc1cc2c4cc5ccccc5cc4c4ccccc4c2cc13 ; CC1(C)c2ccccc2-c2cc3c(cc21)sc1cc2c(cc13)c1ccccc1c1ccc3ccccc3c12 ; c1ccc(-n2c3ccccc3c3cc4c(cc32)sc2cc3c5ccccc5c5c6ccccc6ccc5c3cc24)cc1]

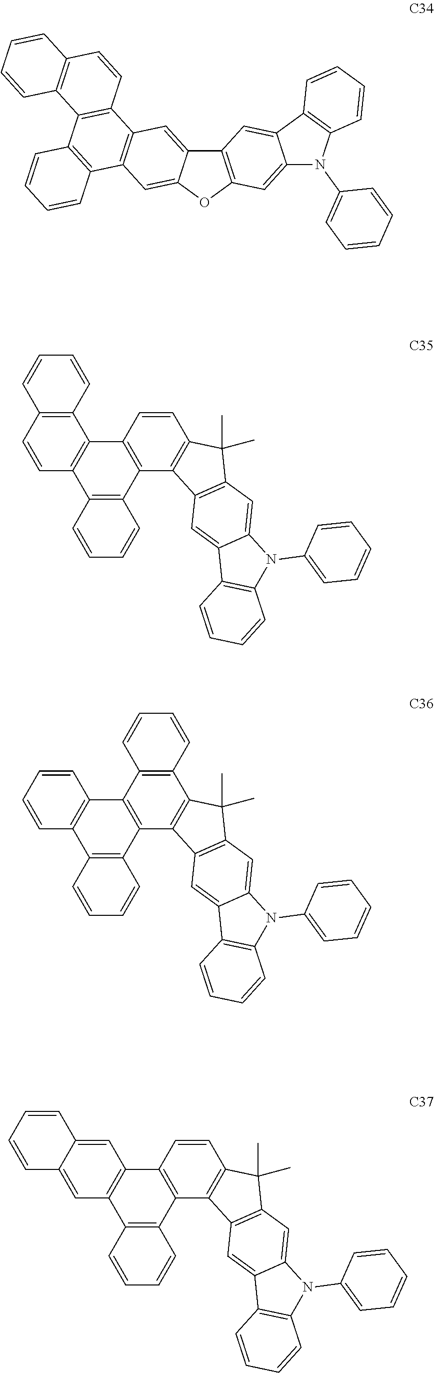 [SMILES: c1ccc(-n2c3ccccc3c3cc4c(cc32)oc2cc3c5ccccc5c5c6ccccc6ccc5c3cc24)cc1 ; CC1(C)c2cc3c(cc2-c2c1ccc1c2c2ccccc2c2ccc4ccccc4c21)c1ccccc1n3-c1ccccc1 ; CC1(C)c2cc3c(cc2-c2c1c1ccccc1c1c4ccccc4c4ccccc4c21)c1ccccc1n3-c1ccccc1 ; CC1(C)c2cc3c(cc2-c2c1ccc1c4cc5ccccc5cc4c4ccccc4c21)c1ccccc1n3-c1ccccc1]

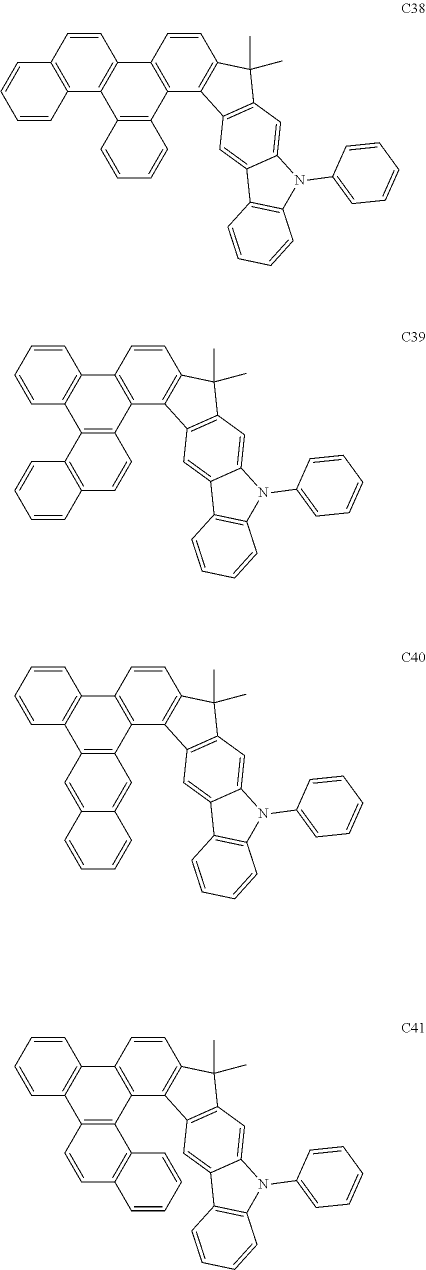 [SMILES: CC1(C)c2cc3c(cc2-c2c1ccc1c4ccc5ccccc5c4c4ccccc4c21)c1ccccc1n3-c1ccccc1 ; CC1(C)c2cc3c(cc2-c2c1ccc1c4ccccc4c4c5ccccc5ccc4c21)c1ccccc1n3-c1ccccc1 ; CC1(C)c2cc3c(cc2-c2c1ccc1c4ccccc4c4cc5ccccc5cc4c21)c1ccccc1n3-c1ccccc1 ; CC1(C)c2cc3c(cc2-c2c1ccc1c4ccccc4c4ccc5ccccc5c4c21)c1ccccc1n3-c1ccccc1]

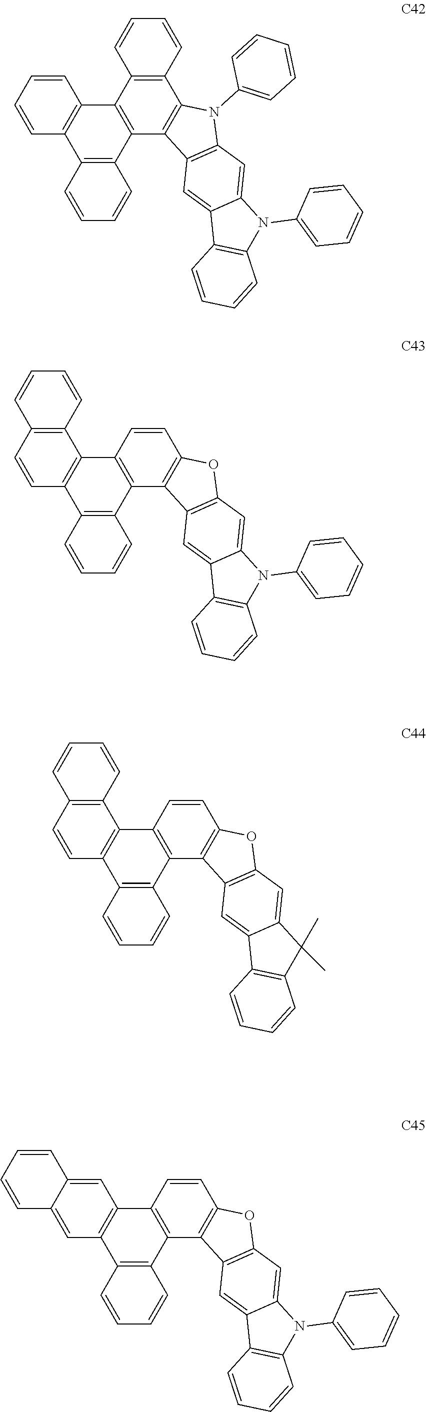 [SMILES: c1ccc(-n2c3ccccc3c3cc4c5c6c7ccccc7c7ccccc7c6c6ccccc6c5n(-c5ccccc5)c4cc32)cc1 ; c1ccc(-n2c3ccccc3c3cc4c(cc32)oc2ccc3c5c6ccccc6ccc5c5ccccc5c3c24)cc1 ; CC1(C)c2ccccc2-c2cc3c(cc21)oc1ccc2c4c5ccccc5ccc4c4ccccc4c2c13 ; c1ccc(-n2c3ccccc3c3cc4c(cc32)oc2ccc3c5cc6ccccc6cc5c5ccccc5c3c24)cc1]

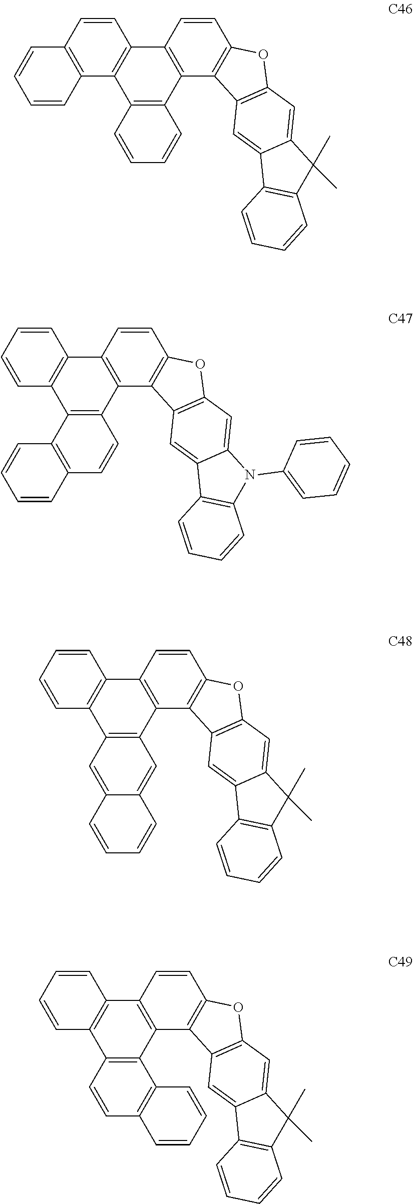 [SMILES: CC1(C)c2ccccc2-c2cc3c(cc21)oc1ccc2c4ccc5ccccc5c4c4ccccc4c2c13 ; c1ccc(-n2c3ccccc3c3cc4c(cc32)oc2ccc3c5ccccc5c5c6ccccc6ccc5c3c24)cc1 ; CC1(C)c2ccccc2-c2cc3c(cc21)oc1ccc2c4ccccc4c4cc5ccccc5cc4c2c13 ; CC1(C)c2ccccc2-c2cc3c(cc21)oc1ccc2c4ccccc4c4ccc5ccccc5c4c2c13]

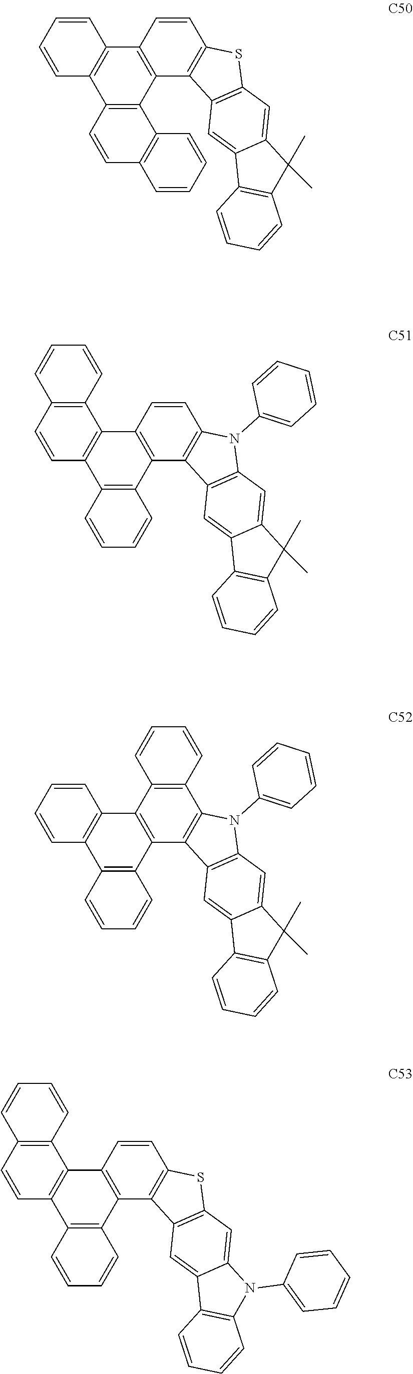 [SMILES: CC1(C)c2ccccc2-c2cc3c(cc21)sc1ccc2c4ccccc4c4ccc5ccccc5c4c2c13 ; CC1(C)c2ccccc2-c2cc3c4c5c6ccccc6c6ccc7ccccc7c6c5ccc4n(-c4ccccc4)c3cc21 ; CC1(C)c2ccccc2-c2cc3c4c5c6ccccc6c6ccccc6c5c5ccccc5c4n(-c4ccccc4)c3cc21 ; c1ccc(-n2c3ccccc3c3cc4c(cc32)sc2ccc3c5c6ccccc6ccc5c5ccccc5c3c24)cc1]

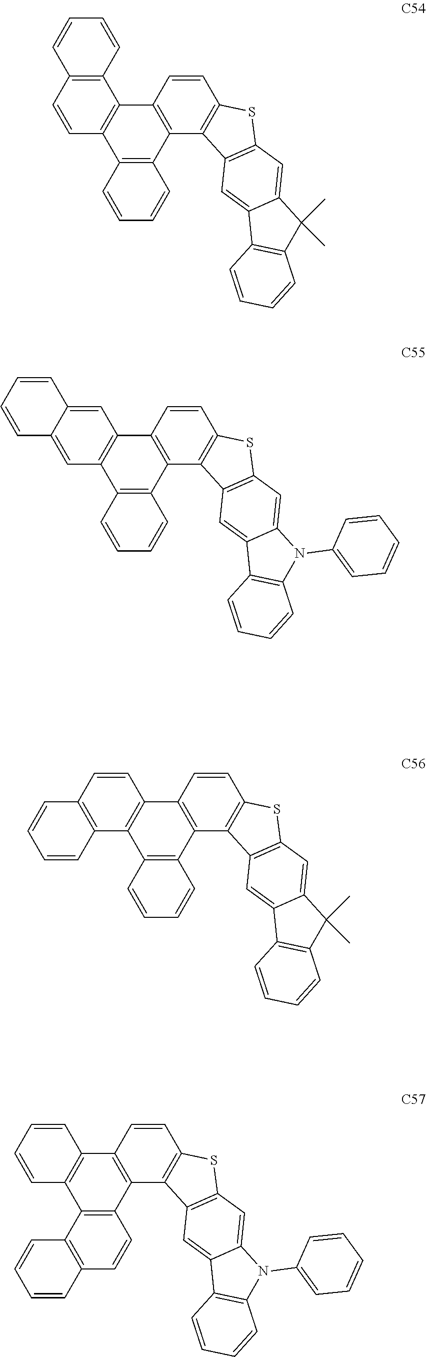 [SMILES: CC1(C)c2ccccc2-c2cc3c(cc21)sc1ccc2c4c5ccccc5ccc4c4ccccc4c2c13 ; c1ccc(-n2c3ccccc3c3cc4c(cc32)sc2ccc3c5cc6ccccc6cc5c5ccccc5c3c24)cc1 ; CC1(C)c2ccccc2-c2cc3c(cc21)sc1ccc2c4ccc5ccccc5c4c4ccccc4c2c13 ; c1ccc(-n2c3ccccc3c3cc4c(cc32)sc2ccc3c5ccccc5c5c6ccccc6ccc5c3c24)cc1]

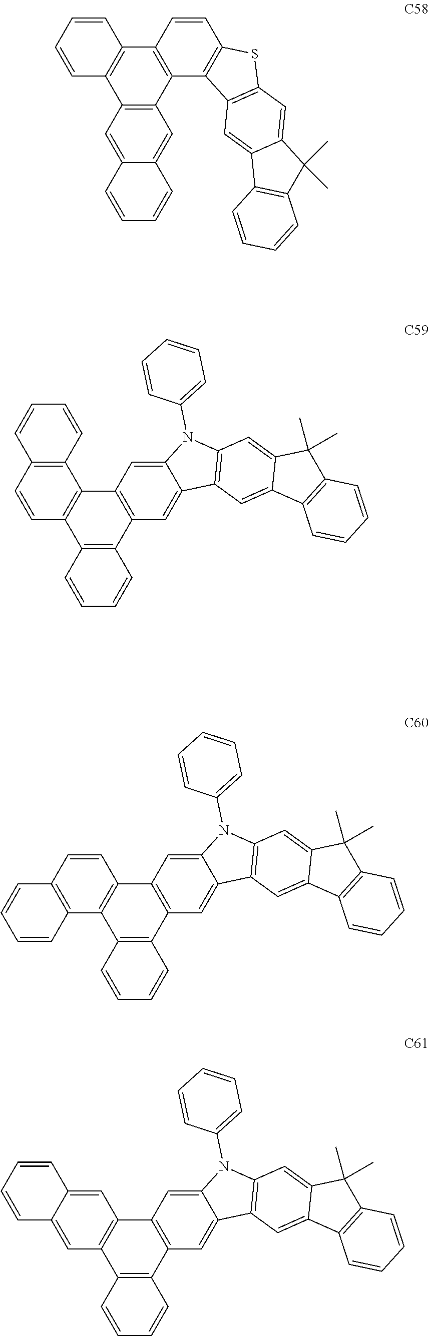 [SMILES: CC1(C)c2ccccc2-c2cc3c(cc21)sc1ccc2c4ccccc4c4cc5ccccc5cc4c2c13 ; CC1(C)c2ccccc2-c2cc3c4cc5c6ccccc6c6ccc7ccccc7c6c5cc4n(-c4ccccc4)c3cc21 ; CC1(C)c2ccccc2-c2cc3c4cc5c6ccccc6c6c7ccccc7ccc6c5cc4n(-c4ccccc4)c3cc21 ; CC1(C)c2ccccc2-c2cc3c4cc5c6ccccc6c6cc7ccccc7cc6c5cc4n(-c4ccccc4)c3cc21]

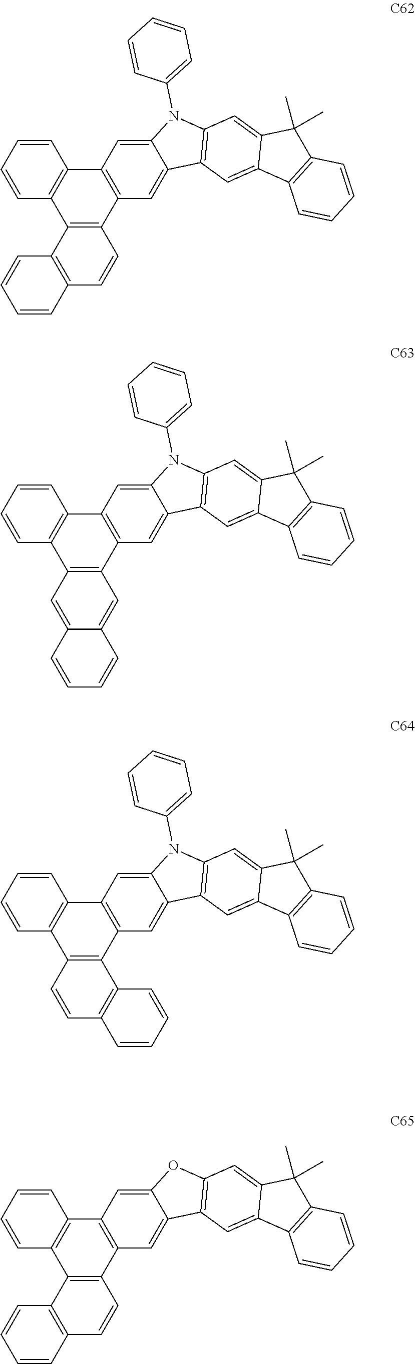 [SMILES: CC1(C)c2ccccc2-c2cc3c4cc5c(cc4n(-c4ccccc4)c3cc21)c1ccccc1c1c2ccccc2ccc51 ; CC1(C)c2ccccc2-c2cc3c4cc5c6cc7ccccc7cc6c6ccccc6c5cc4n(-c4ccccc4)c3cc21 ; CC1(C)c2ccccc2-c2cc3c4cc5c(cc4n(-c4ccccc4)c3cc21)c1ccccc1c1ccc2ccccc2c15 ; CC1(C)c2ccccc2-c2cc3c(cc21)oc1cc2c4ccccc4c4c5ccccc5ccc4c2cc13]

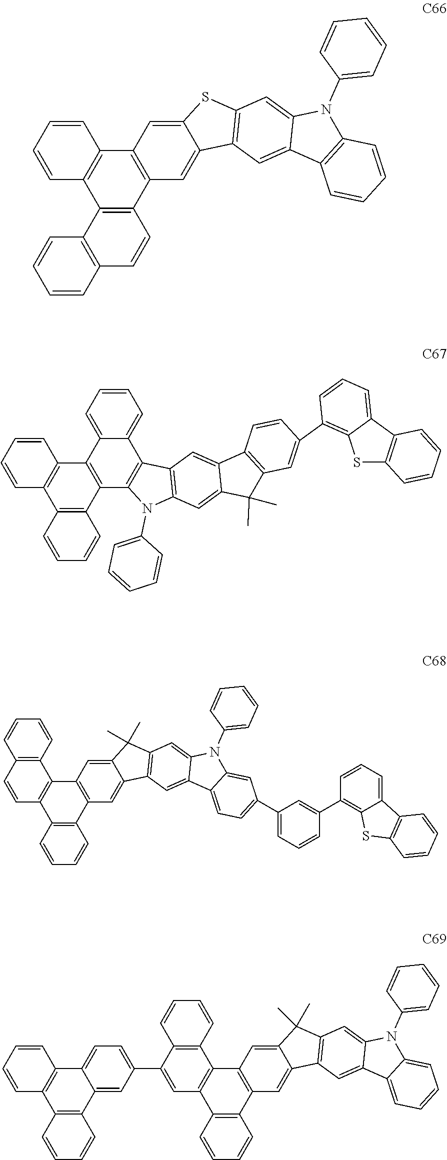 [SMILES: c1ccc(-n2c3ccccc3c3cc4c(cc32)sc2cc3c5ccccc5c5c6ccccc6ccc5c3cc24)cc1 ; CC1(C)c2cc(-c3cccc4c3sc3ccccc34)ccc2-c2cc3c4c5ccccc5c5c6ccccc6c6ccccc6c5c4n(-c4ccccc4)c3cc21 ; CC1(C)c2cc3c(cc2-c2cc4c5ccc(-c6cccc(-c7cccc8c7sc7ccccc78)c6)cc5n(-c5ccccc5)c4cc21)c1ccccc1c1ccc2ccccc2c13 ; CC1(C)c2cc3c(cc2-c2cc4c5ccccc5n(-c5ccccc5)c4cc21)c1ccccc1c1cc(-c2ccc4c5ccccc5c5ccccc5c4c2)c2ccccc2c31]

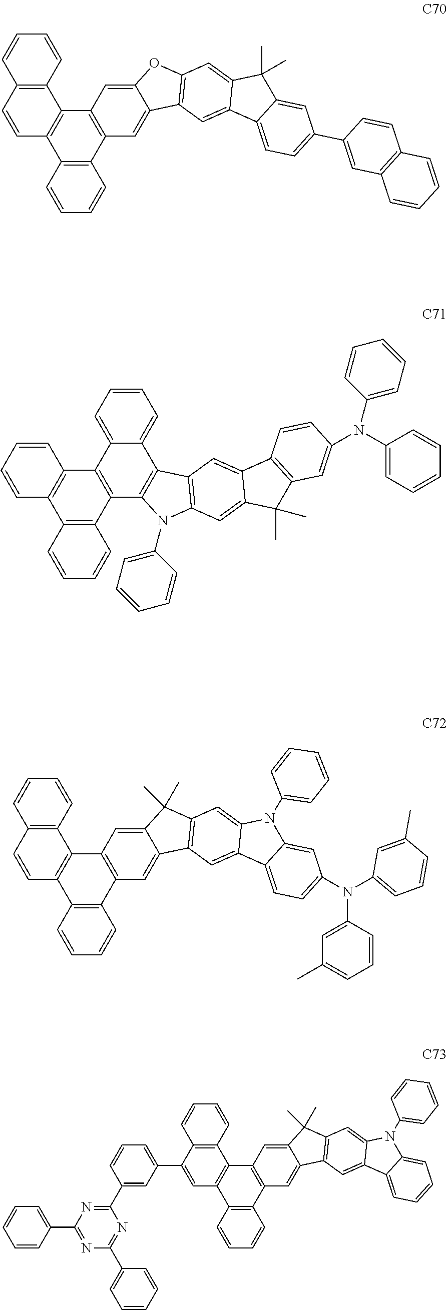 [SMILES: CC1(C)c2cc(-c3ccc4ccccc4c3)ccc2-c2cc3c(cc21)oc1cc2c(cc13)c1ccccc1c1ccc3ccccc3c12 ; CC1(C)c2cc(N(c3ccccc3)c3ccccc3)ccc2-c2cc3c4c5ccccc5c5c6ccccc6c6ccccc6c5c4n(-c4ccccc4)c3cc21 ; Cc1cccc(N(c2cccc(C)c2)c2ccc3c4cc5c(cc4n(-c4ccccc4)c3c2)C(C)(C)c2cc3c(cc2-5)c2ccccc2c2ccc4ccccc4c23)c1 ; CC1(C)c2cc3c(cc2-c2cc4c5ccccc5n(-c5ccccc5)c4cc21)c1ccccc1c1cc(-c2cccc(-c4nc(-c5ccccc5)nc(-c5ccccc5)n4)c2)c2ccccc2c31]

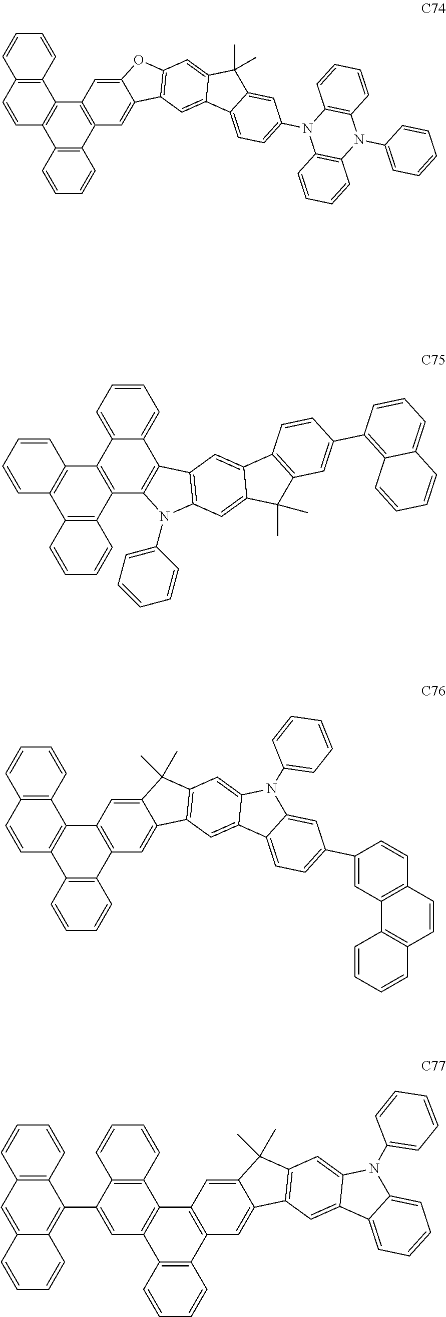 [SMILES: CC1(C)c2cc(N3c4ccccc4N(c4ccccc4)c4ccccc43)ccc2-c2cc3c(cc21)oc1cc2c(cc13)c1ccccc1c1ccc3ccccc3c12 ; CC1(C)c2cc(-c3cccc4ccccc34)ccc2-c2cc3c4c5ccccc5c5c6ccccc6c6ccccc6c5c4n(-c4ccccc4)c3cc21 ; CC1(C)c2cc3c(cc2-c2cc4c5ccc(-c6ccc7ccc8ccccc8c7c6)cc5n(-c5ccccc5)c4cc21)c1ccccc1c1ccc2ccccc2c13 ; CC1(C)c2cc3c(cc2-c2cc4c5ccccc5n(-c5ccccc5)c4cc21)c1ccccc1c1cc(-c2c4ccccc4cc4ccccc24)c2ccccc2c31]

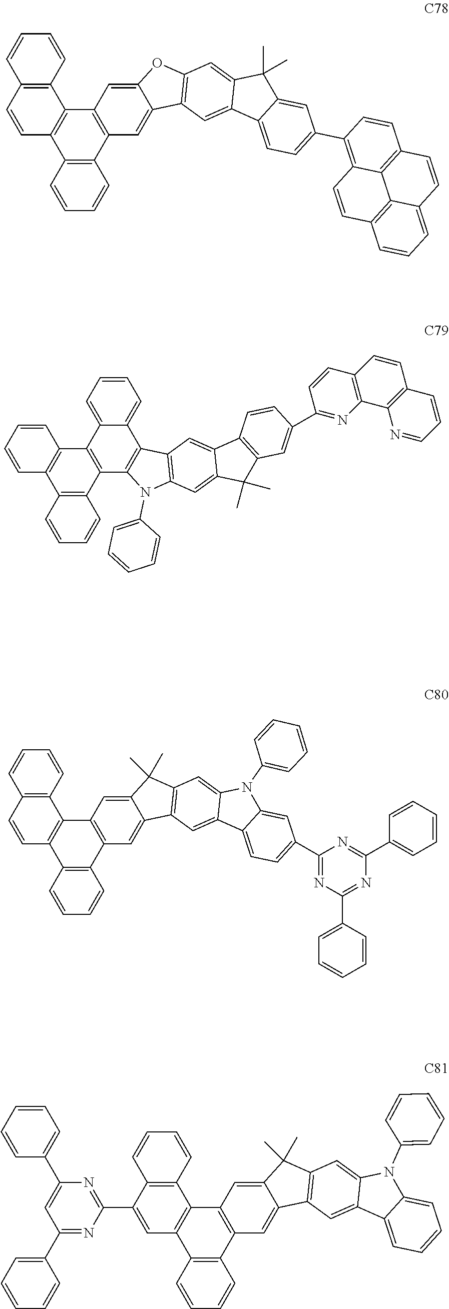 [SMILES: CC1(C)c2cc(-c3ccc4ccc5cccc6ccc3c4c56)ccc2-c2cc3c(cc21)oc1cc2c(cc13)c1ccccc1c1ccc3ccccc3c12 ; CC1(C)c2cc(-c3ccc4ccc5cccnc5c4n3)ccc2-c2cc3c4c5ccccc5c5c6ccccc6c6ccccc6c5c4n(-c4ccccc4)c3cc21 ; CC1(C)c2cc3c(cc2-c2cc4c5ccc(-c6nc(-c7ccccc7)nc(-c7ccccc7)n6)cc5n(-c5ccccc5)c4cc21)c1ccccc1c1ccc2ccccc2c13 ; CC1(C)c2cc3c(cc2-c2cc4c5ccccc5n(-c5ccccc5)c4cc21)c1ccccc1c1cc(-c2nc(-c4ccccc4)cc(-c4ccccc4)n2)c2ccccc2c31]

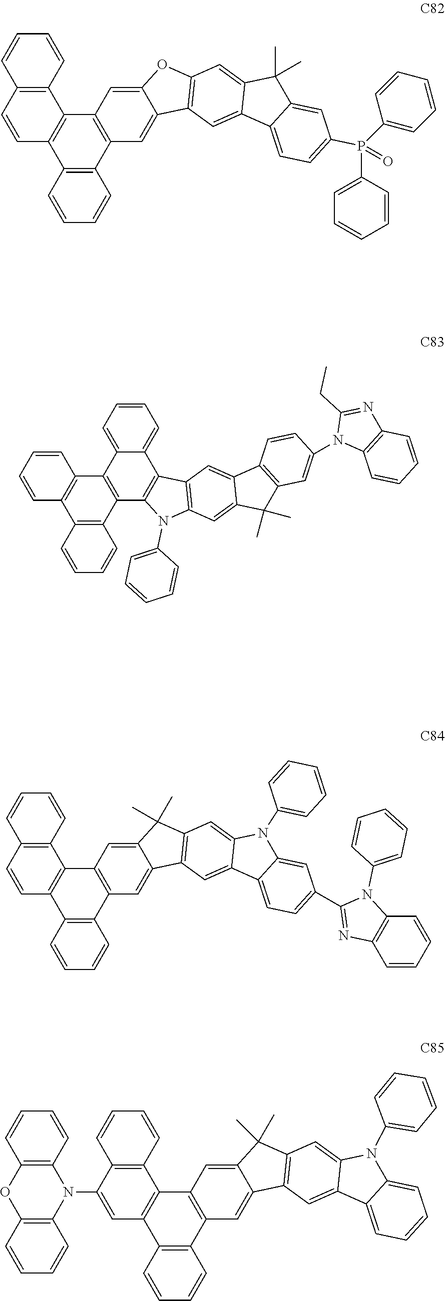 [SMILES: CC1(C)c2cc(P(=O)(c3ccccc3)c3ccccc3)ccc2-c2cc3c(cc21)oc1cc2c(cc13)c1ccccc1c1ccc3ccccc3c12 ; CCc1nc2ccccc2n1-c1ccc2c(c1)C(C)(C)c1cc3c(cc1-2)c1c2ccccc2c2c4ccccc4c4ccccc4c2c1n3-c1ccccc1 ; CC1(C)c2cc3c(cc2-c2cc4c5ccc(-c6nc7ccccc7n6-c6ccccc6)cc5n(-c5ccccc5)c4cc21)c1ccccc1c1ccc2ccccc2c13 ; CC1(C)c2cc3c(cc2-c2cc4c5ccccc5n(-c5ccccc5)c4cc21)c1ccccc1c1cc(N2c4ccccc4Oc4ccccc42)c2ccccc2c31]

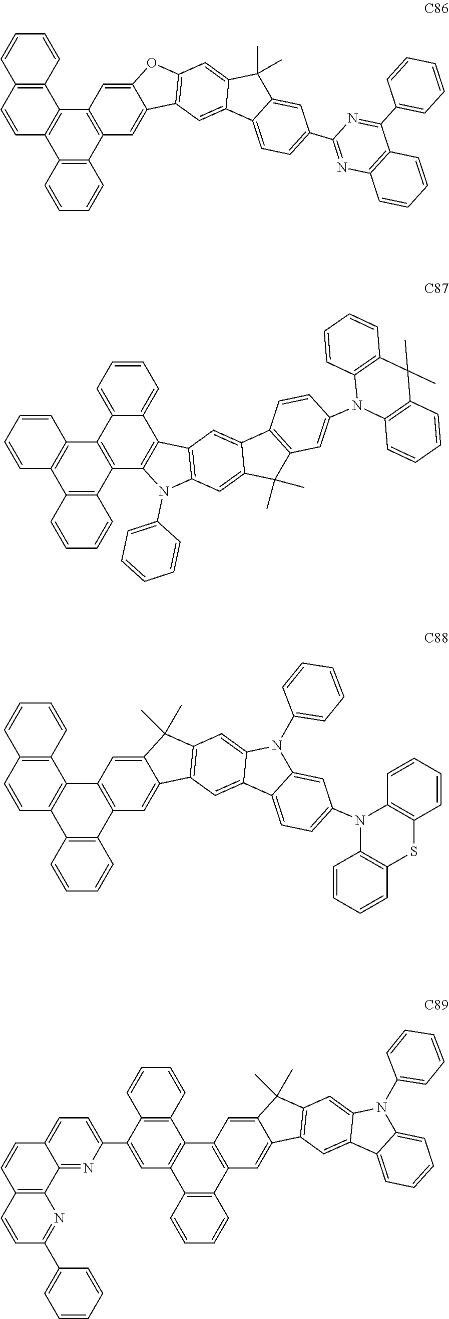 [SMILES: CC1(C)c2cc(-c3nc(-c4ccccc4)c4ccccc4n3)ccc2-c2cc3c(cc21)oc1cc2c(cc13)c1ccccc1c1ccc3ccccc3c12 ; CC1(C)c2cc(N3c4ccccc4C(C)(C)c4ccccc43)ccc2-c2cc3c4c5ccccc5c5c6ccccc6c6ccccc6c5c4n(-c4ccccc4)c3cc21 ; CC1(C)c2cc3c(cc2-c2cc4c5ccc(N6c7ccccc7Sc7ccccc76)cc5n(-c5ccccc5)c4cc21)c1ccccc1c1ccc2ccccc2c13 ; CC1(C)c2cc3c(cc2-c2cc4c5ccccc5n(-c5ccccc5)c4cc21)c1ccccc1c1cc(-c2ccc4ccc5ccc(-c6ccccc6)nc5c4n2)c2ccccc2c31]

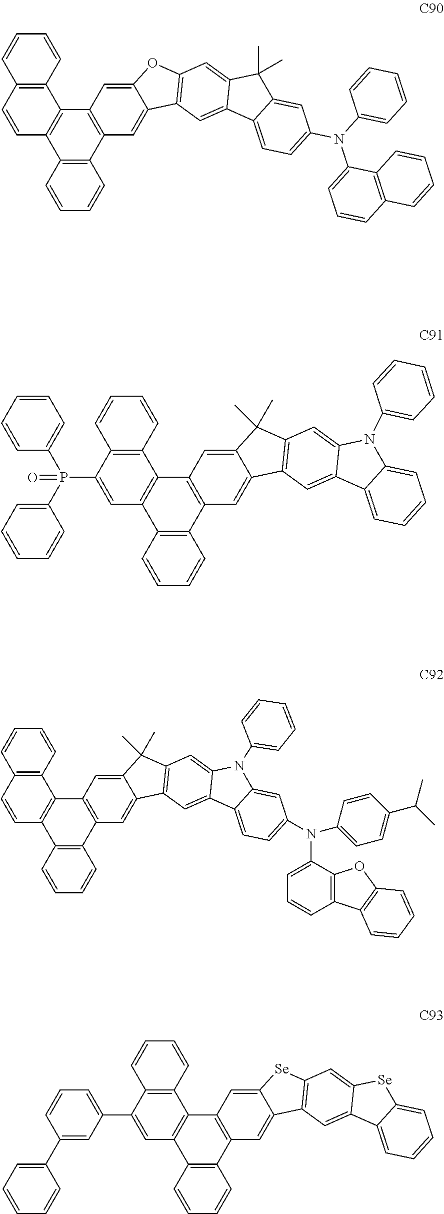 [SMILES: CC1(C)c2cc(N(c3ccccc3)c3cccc4ccccc34)ccc2-c2cc3c(cc21)oc1cc2c(cc13)c1ccccc1c1ccc3ccccc3c12 ; CC1(C)c2cc3c(cc2-c2cc4c5ccccc5n(-c5ccccc5)c4cc21)c1ccccc1c1cc(P(=O)(c2ccccc2)c2ccccc2)c2ccccc2c31 ; CC(C)c1ccc(N(c2ccc3c4cc5c(cc4n(-c4ccccc4)c3c2)C(C)(C)c2cc3c(cc2-5)c2ccccc2c2ccc4ccccc4c23)c2cccc3c2oc2ccccc23)cc1 ; c1ccc(-c2cccc(-c3cc4c5ccccc5c5cc6c(cc5c4c4ccccc34)[se]c3cc4[se]c5ccccc5c4cc36)c2)cc1]

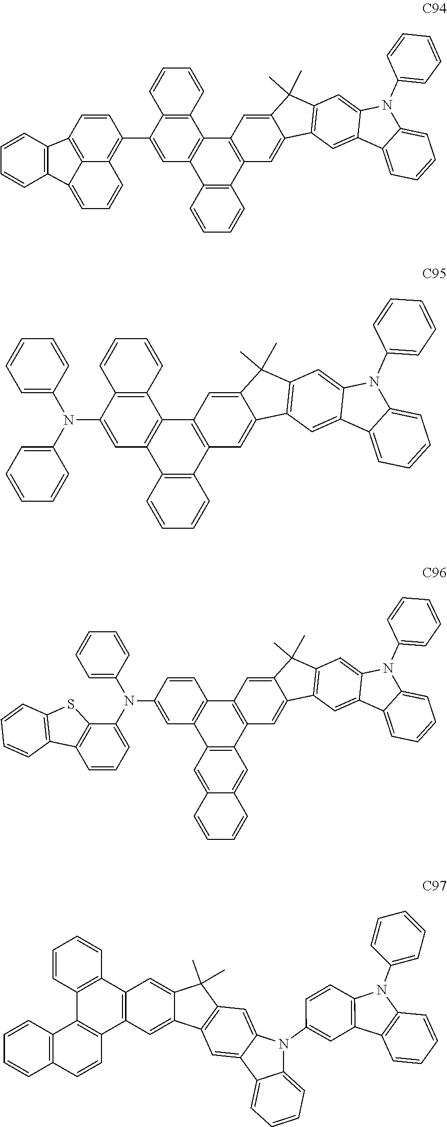 [SMILES: CC1(C)c2cc3c(cc2-c2cc4c5ccccc5n(-c5ccccc5)c4cc21)c1ccccc1c1cc(-c2ccc4c5c(cccc25)-c2ccccc2-4)c2ccccc2c31 ; CC1(C)c2cc3c(cc2-c2cc4c5ccccc5n(-c5ccccc5)c4cc21)c1ccccc1c1cc(N(c2ccccc2)c2ccccc2)c2ccccc2c31 ; CC1(C)c2cc3c4ccc(N(c5ccccc5)c5cccc6c5sc5ccccc56)cc4c4cc5ccccc5cc4c3cc2-c2cc3c4ccccc4n(-c4ccccc4)c3cc21 ; CC1(C)c2cc3c4ccccc4c4c5ccccc5ccc4c3cc2-c2cc3c4ccccc4n(-c4ccc5c(c4)c4ccccc4n5-c4ccccc4)c3cc21]

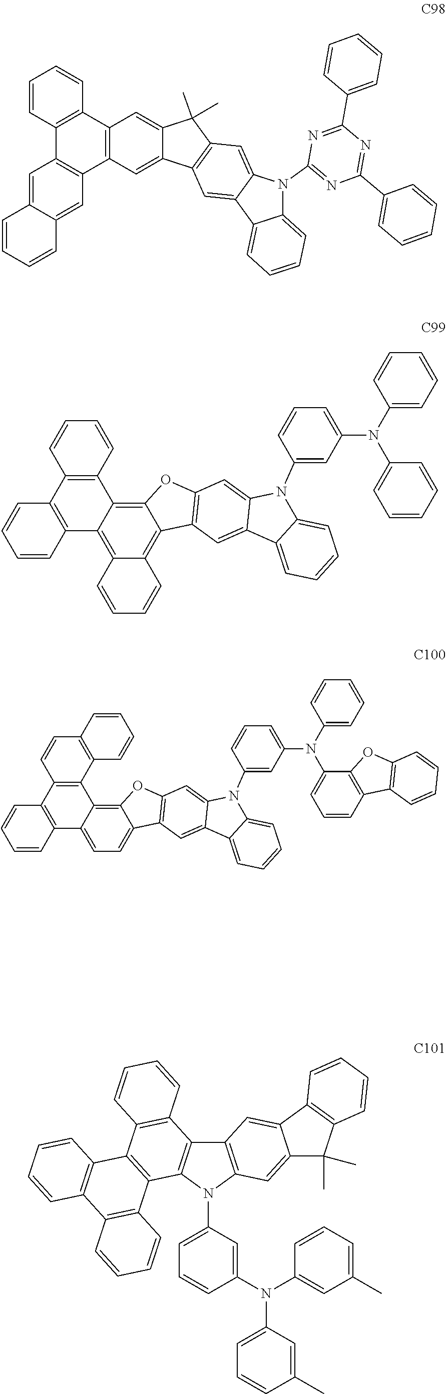 [SMILES: CC1(C)c2cc3c4ccccc4c4cc5ccccc5cc4c3cc2-c2cc3c4ccccc4n(-c4nc(-c5ccccc5)nc(-c5ccccc5)n4)c3cc21 ; c1ccc(N(c2ccccc2)c2cccc(-n3c4ccccc4c4cc5c(cc43)oc3c5c4ccccc4c4c5ccccc5c5ccccc5c34)c2)cc1 ; c1ccc(N(c2cccc(-n3c4ccccc4c4cc5c(cc43)oc3c5ccc4c5ccccc5c5ccc6ccccc6c5c43)c2)c2cccc3c2oc2ccccc23)cc1 ; Cc1cccc(N(c2cccc(C)c2)c2cccc(-n3c4cc5c(cc4c4c6ccccc6c6c7ccccc7c7ccccc7c6c43)-c3ccccc3C5(C)C)c2)c1]

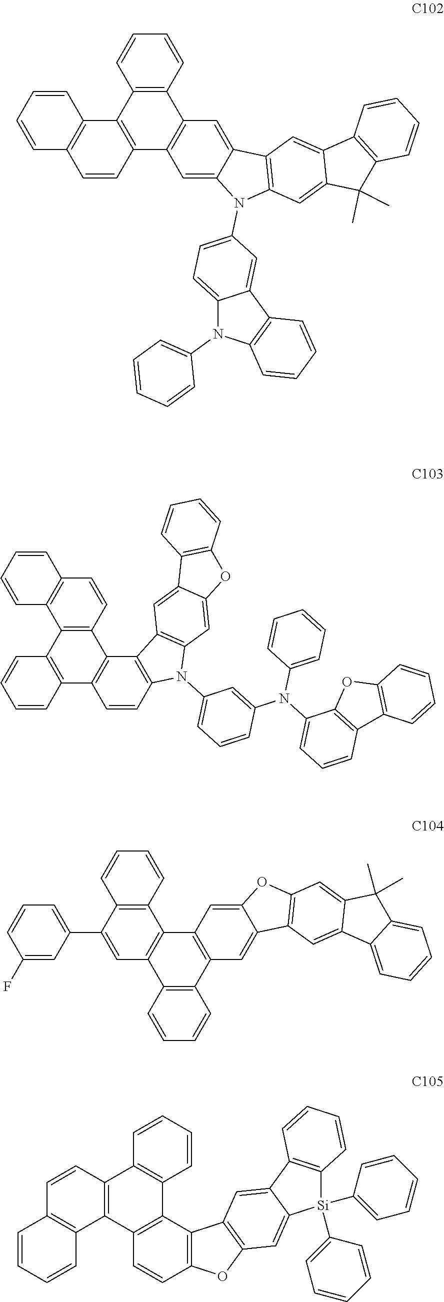 [SMILES: CC1(C)c2ccccc2-c2cc3c4cc5c6ccccc6c6c7ccccc7ccc6c5cc4n(-c4ccc5c(c4)c4ccccc4n5-c4ccccc4)c3cc21 ; c1ccc(N(c2cccc(-n3c4cc5oc6ccccc6c5cc4c4c5c(ccc43)c3ccccc3c3c4ccccc4ccc35)c2)c2cccc3c2oc2ccccc23)cc1 ; CC1(C)c2ccccc2-c2cc3c(cc21)oc1cc2c(cc13)c1ccccc1c1cc(-c3cccc(F)c3)c3ccccc3c12 ; c1ccc([Si]2(c3ccccc3)c3ccccc3-c3cc4c(cc32)oc2ccc3c5c6ccccc6ccc5c5ccccc5c3c24)cc1]

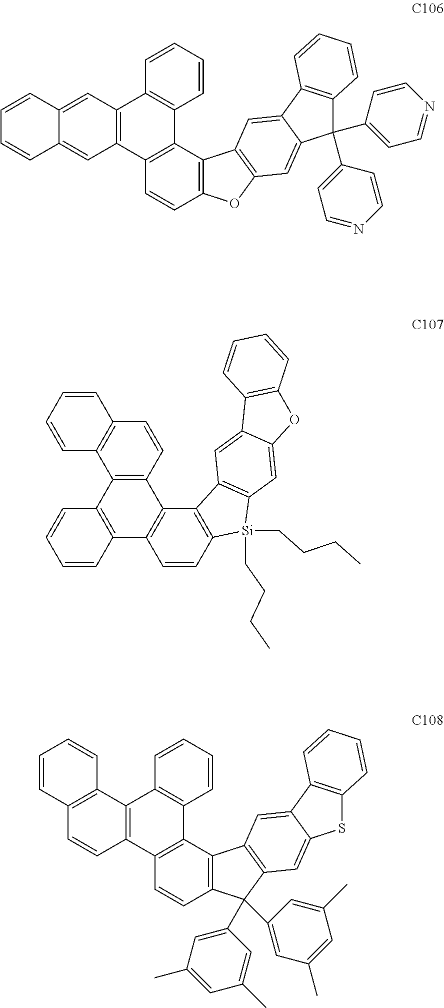 [SMILES: c1ccc2c(c1)-c1cc3c(cc1C2(c1ccncc1)c1ccncc1)oc1ccc2c4cc5ccccc5cc4c4ccccc4c2c13 ; CCCC[Si]1(CCCC)c2cc3oc4ccccc4c3cc2-c2c1ccc1c3ccccc3c3c4ccccc4ccc3c21 ; Cc1cc(C)cc(C2(c3cc(C)cc(C)c3)c3cc4sc5ccccc5c4cc3-c3c2ccc2c4ccc5ccccc5c4c4ccccc4c32)c1]

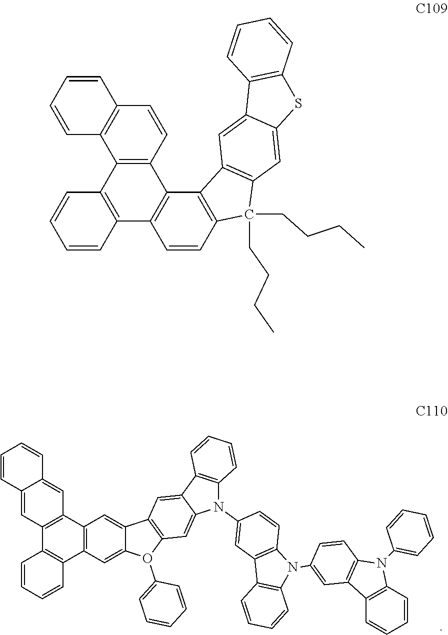

In another embodiment of the present invention, an organic electroluminescence device is disclosed. The organic electroluminescence device comprises a pair of electrodes composed of a cathode and an anode, and a light emitting layer and one or more organic thin film layers between the pair of electrodes. At least one of the light emitting layer and the organic thin film layer comprises the organic compound of formula (1) or formula (2).

In some embodiments, the light emitting layer comprising the organic compound of formula (1) or formula (2) is a host material. The host material may be a phosphorescent host material or a fluorescent host material. In certain embodiments, the light emitting layer comprising the organic compound of formula (1) or formula (2) is used as a fluorescent dopant material.

In some embodiments, the organic thin film layer comprising the organic compound of formula (1) or formula (2) is an electron transporting layer.

In a further embodiment of the present invention, the organic electroluminescence device is a lighting panel. In other embodiment of the present invention, the organic electroluminescence device is a backlight panel.

Detailed preparation of the organic compounds of the present invention will be clarified by exemplary embodiments below, but the present invention is not limited thereto. EXAMPLES 1 to 15 show the preparation of the organic compounds of the present invention, and EXAMPLES 16 to 18 show the fabrication and test reports of the organic EL devices.

### Example 1

### Synthesis of Intermediate A1

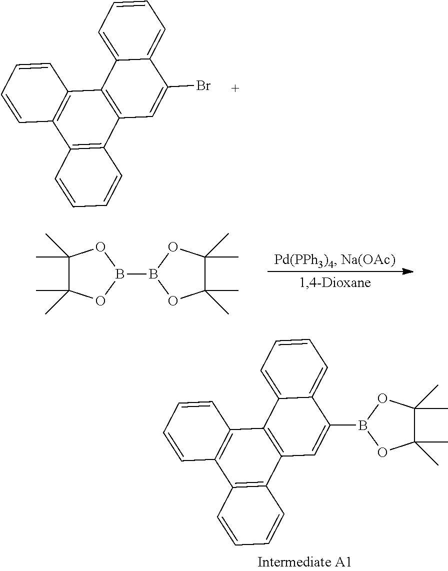

A mixture of 3 g (8.4 mmol) of 10-bromobenzo[g]chrysene, 2.5 g (10.1 mmol) of 4,4,4′,4′,5,5,5′,5′-octamethyl-2,2′-bi(1,3,2-dioxaborolane), 0.12 g (0.1 mmol) of Pd(PPh₃)₄, 1.0 g (12.6 mmol) of sodium acetate, and 60 ml of 1,4-dioxane was degassed and placed under nitrogen, and then heated at 100° C. for 6 hrs. After the reaction finished, the mixture was allowed to cool to room temperature. Subsequently, the organic layer was extracted with ethyl acetate and water, and then dried with anhydrous magnesium sulfate. After the solvent was removed, the residue was purified by column chromatography on silica to give Intermediate A1 (2.8 g, 85%).

### Synthesis of Intermediate A2

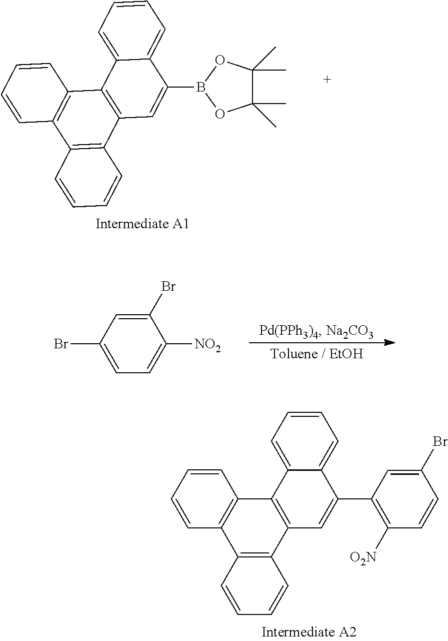 [SMILES: CC1(C)OB(c2cc3c4ccccc4c4ccccc4c3c3ccccc23)OC1(C)C ; O=[N+]([O-])c1ccc(Br)cc1Br ; O=[N+]([O-])c1ccc(Br)cc1-c1cc2c3ccccc3c3ccccc3c2c2ccccc12]

A mixture of 2 g (5.0 mmol) of Intermediate A1, 1.4 g (5.0 mmol) of 2,4-dibromonitrobenzene, 0.06 g (0.05 mmol) of Pd(PPh₃)₄, 10 ml of 2M Na₂CO_{3(aq)}, 10 ml of EtOH, and 30 ml of toluene was degassed and placed under nitrogen, and then heated at 100° C. for 12 hrs. After the reaction finished, the mixture was allowed to cool to room temperature. Subsequently, the organic layer was extracted with dichloromethane and water, and then dried with anhydrous MgSO₄. After the solvent was removed, the residue was purified by column chromatography on silica to give Intermediate A2 (1.2 g, 50%).

### Synthesis of Intermediate A3

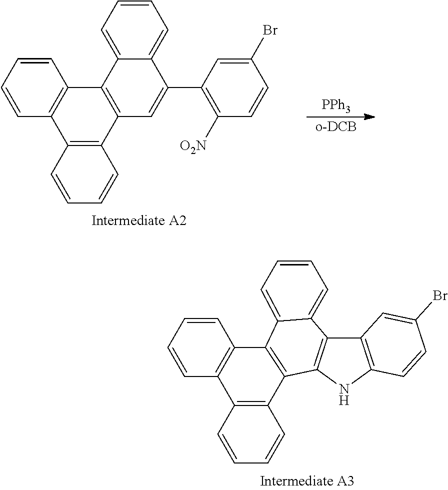 [SMILES: O=[N+]([O-])c1ccc(Br)cc1-c1cc2c3ccccc3c3ccccc3c2c2ccccc12 ; "o-DCB" ; Brc1ccc2[nH]c3c(c2c1)c1ccccc1c1c2ccccc2c2ccccc2c31 ; "PPh3"]

A mixture of 1 g (2.1 mmol) of Intermediate A2, 5.5 g (21.0 mmol) of Triphenylphosphine, and 30 ml of oDCB was placed under nitrogen gas, and then heated at 180° C. for 8 hrs. After the reaction finished, the mixture was allowed to cool to room temperature. The mixture was poured into water, and then filtered to give Intermediate A3 (0.5 g, 50%).

### Synthesis of Intermediate A4

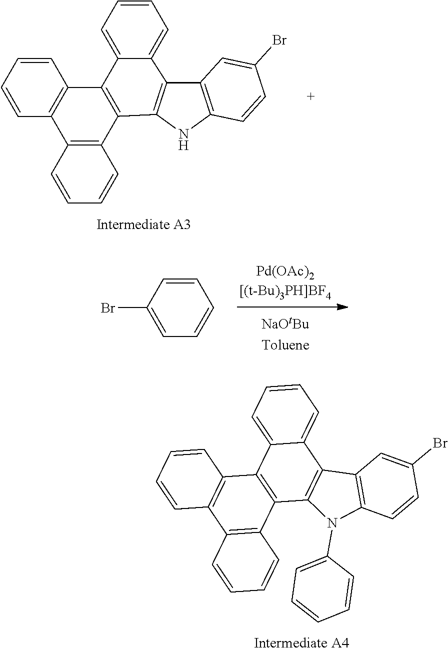

A mixture of 2.0 g (4.5 mmol) of Intermediate A3, 1.1 g (6.7 mmol) of bromobenzene, 0.05 g (0.2 mmol) of Pd(OAc)₂, 0.1 g (0.4 mmol) of tri-tert-butylphosphonium tetrafluoroborate, 0.9 g (9.0 mmol) of sodium tert-butoxide, and 50 ml of toluene was degassed and placed under nitrogen gas, and then heated at 120° C. for 16 hrs. After the reaction finished, the mixture was allowed to cool to room temperature. Subsequently, the organic layer was extracted with dichloromethane and water, and then dried with anhydrous MgSO₄. After the solvent was removed, the residue was purified by column chromatography on silica to give Intermediate A4 (1.3 g, 55%). MS(m/z, FAB+): 523.5.

### Synthesis of Intermediate A5

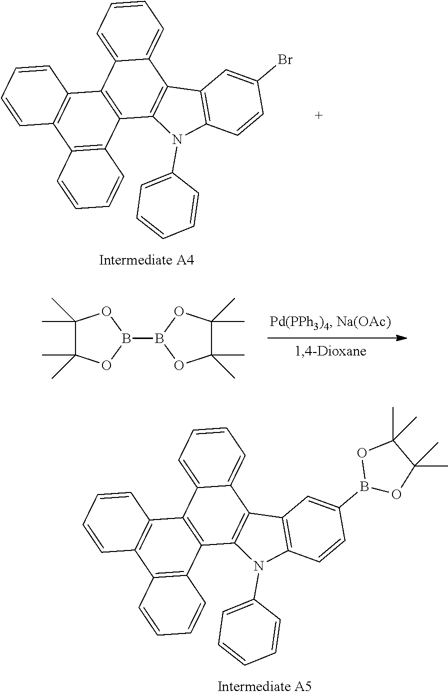

A mixture of 2 g (3.8 mmol) of Intermediate A4, 1.5 g (5.7 mmol) of 4,4,4′,4′,5,5,5′,5′-octamethyl-2,2′-bi(1,3,2-dioxaborolane), 0.09 g (0.076 mmol) of Pd(PPh₃)₄, 0.7 g (7.6 mmol) of sodium acetate, and 60 ml of 1,4-dioxane was degassed and placed under nitrogen, and then heated at 100° C. for 6 hrs. After the reaction finished, the mixture was allowed to cool to room temperature. Subsequently, the organic layer was extracted with ethyl acetate and water, and then dried with anhydrous magnesium sulfate. After the solvent was removed, the residue was purified by column chromatography on silica to give Intermediate A5 (1.7 g, 80%).

### Synthesis of Intermediate A6

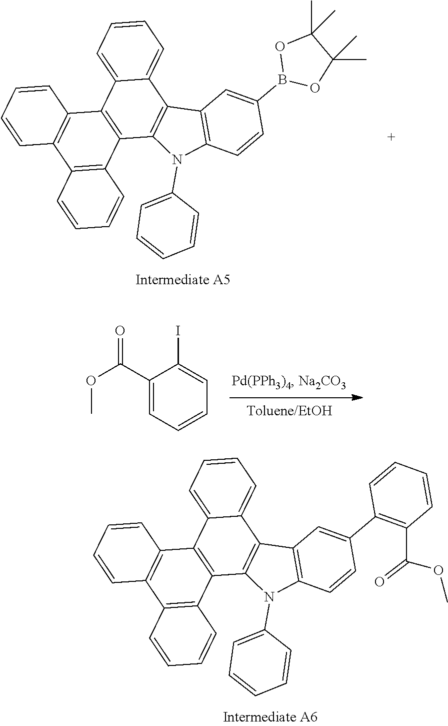

A mixture of 2 g (3.5 mmol) of Intermediate A5, 1.0 g (3.5 mmol) of methyl 2-iodobenzoate, 0.04 g (0.04 mmol) of Pd(PPh₃)₄, 10 ml of 2M Na₂CO_{3(aq)}, 10 ml of EtOH, and 30 ml of toluene was degassed and placed under nitrogen, and then heated at 100° C. for 12 hrs. After the reaction finished, the mixture was allowed to cool to room temperature. Subsequently, the organic layer was extracted with dichloromethane and water, and then dried with anhydrous MgSO₄. After the solvent was removed, the residue was purified by column chromatography on silica to give Intermediate A6 (1.6 g, 82.1%).

### Synthesis of Compound C1

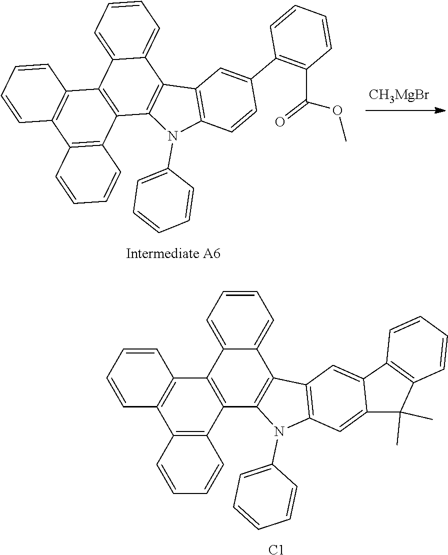

Under the nitrogen gas, while 1.6 g (2.8 mmol) of Intermediate A6 was stirred in dry THF, methyl magnesium bromide (6 equivalent) was slowly added dropwise thereto. The mixture was stirred for 16 hrs at room temperature. After completion of the reaction, a little distilled water was slowly added, and then the mixture was extracted with ethyl acetate and washed with water sequentially. The organic layer was then dried with anhydrous MgSO₄ to remove the water for obtaining a residue. Subsequently, excess phosphoric acid solvent (˜10 ml) was added to the residue, which was then stirred at room temperature for more than 16 hrs. Afterwards, distilled water (˜50 ml) was slowly added and then stirred for 1 hour. After the precipitated solids were filtered, the filtered solids were extracted with dichloromethane solvent and then washed with sodium hydroxide aqueous solution. Subsequently, the dichloromethane solvent layer was taken out and then the moisture was removed by using magnesium sulfate. Finally, the residual solvent was removed to obtain the Compound C1 (0.6 g, 43%). MS(m/z, FAB+): 559.25.

### Example 2

### Synthesis of Intermediate A7

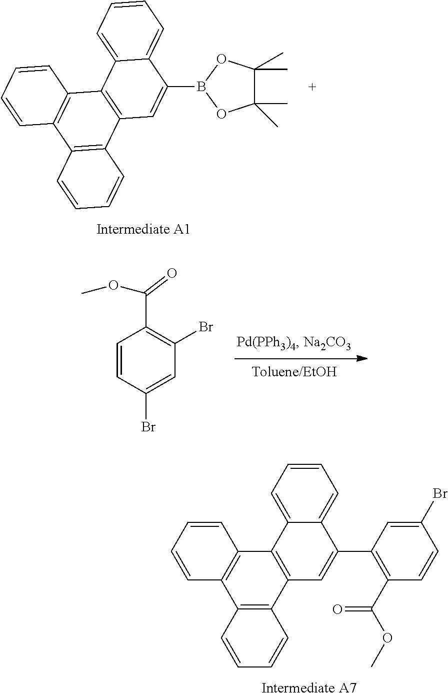

The same synthesis procedure as in Synthesis of Intermediate A6 was used, except that 2 g of Intermediate A1 was used instead of Intermediate A5 and 1.4 g of methyl 2,4-dibromobenzoate was used instead of methyl 2-iodobenzoate to obtain the desired Intermediate A7 (1.9 g, yield=81%).

### Synthesis of Intermediate A8

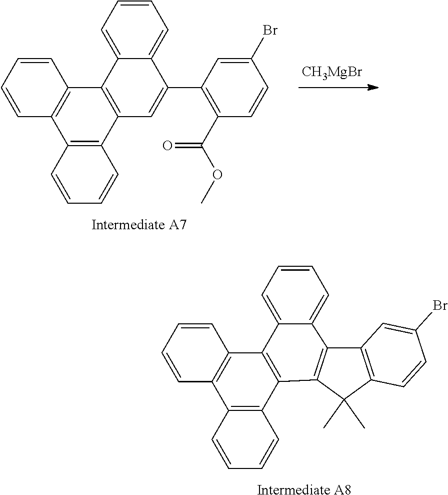

The same synthesis procedure as in Synthesis of Compound C1 was used, except that 1.9 g of Intermediate A7 was used instead of Intermediate A6 to obtain the desired Intermediate A8 (1.1 g, yield=61%).

### Synthesis of Intermediate A9

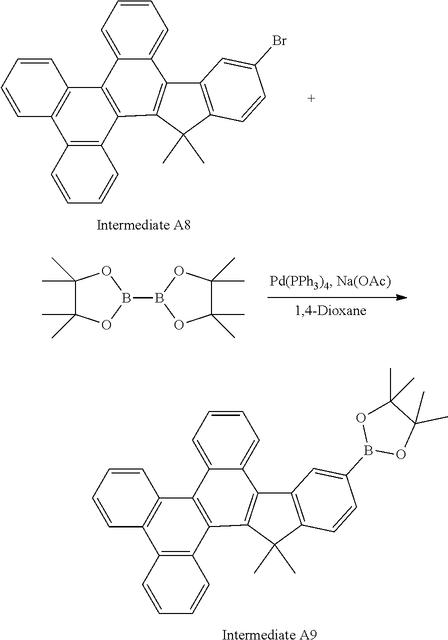

The same synthesis procedure as in Synthesis of Intermediate A1 was used, except that 3 g of Intermediate A8 was used instead of 10-bromobenzo[g]chrysene to obtain the desired Intermediate A9 (2.3 g, yield=72.1%).

### Synthesis of Intermediate A10

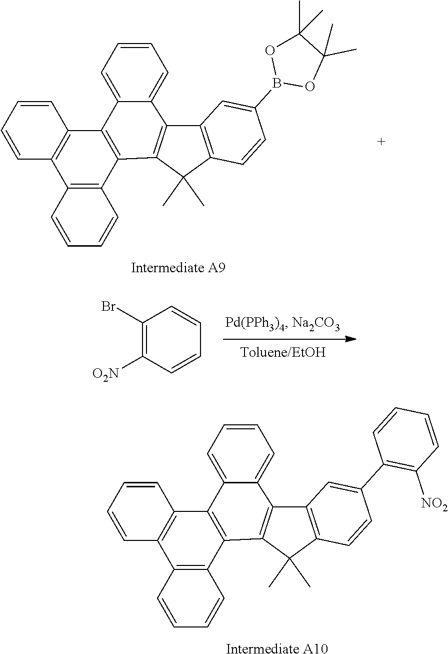

The same synthesis procedure as in Synthesis of Intermediate A2 was used, except that 2 g of Intermediate A9 was used instead of Intermediate A1 and 1.4 g of 1-bromo-2-nitrobenzene was used instead of 2,4-dibromonitrobenzene to obtain the desired Intermediate A10 (1.0 g, yield=53%).

### Synthesis of Intermediate A11

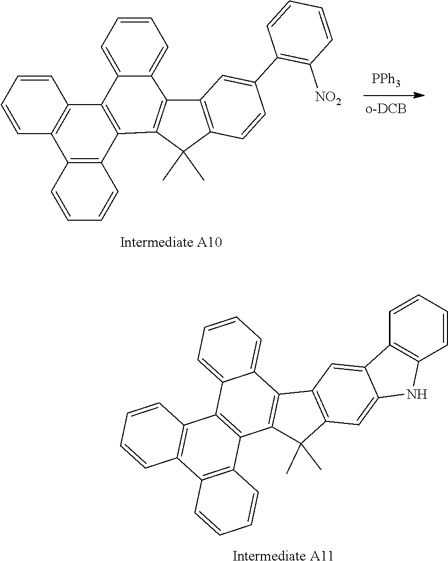

The same synthesis procedure as in Synthesis of Intermediate A3 was used, except that 3 g of Intermediate A10 was used instead of Intermediate A2 to obtain the desired Intermediate A11 (1.9 g, yield=67.6%).

### Synthesis of Compound C2

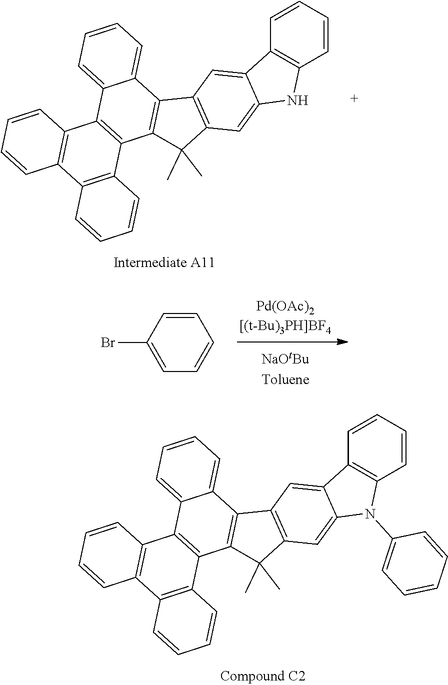

The same synthesis procedure as in Synthesis of Intermediate A4 was used, except that 2 g of Intermediate A11 was used instead of Intermediate A3 to obtain the desired Compound C2 (1.2 g, yield=53%).

### Example 3

### Synthesis of Intermediate A12

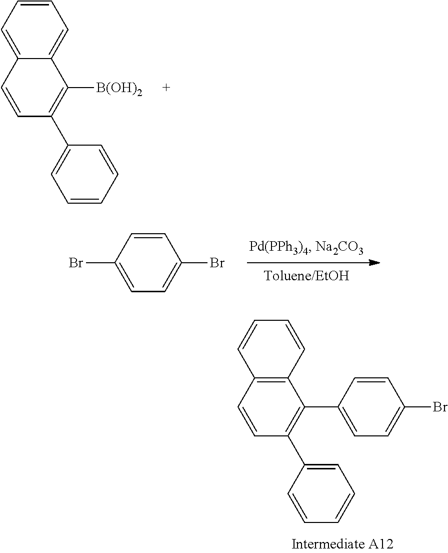

The same synthesis procedure as in Synthesis of Intermediate A2 was used, except that 3 g of 2-phenylnaphthalen-1-ylboronic acid was used instead of Intermediate A1 and 2.8 g of 1,4-dibromobenzene was used instead of 2,4-dibromonitrobenzene to obtain the desired Intermediate A12 (2.1 g, yield=50%).

### Synthesis of Intermediate A13

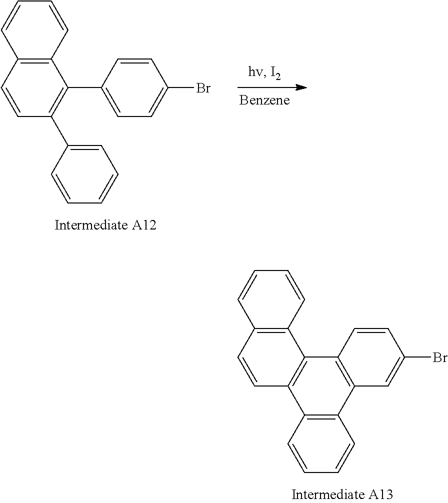 [SMILES: Brc1ccc(-c2c(-c3ccccc3)ccc3ccccc23)cc1 ; Brc1ccc2c(c1)c1ccccc1c1ccc3ccccc3c12]

A mixture of 10 g (27.8 mmol) of Intermediate A12, 0.07 g (0.28 mmol) of Iodine, and 1000 ml of benzene was degassed and placed under nitrogen, and then exposed to UV light for 4 hrs. After the reaction finished, the solvent was removed, and then the residue was recrystallized 3 times to give Intermediate A13 (2.3 g, 23%).

### Synthesis of Intermediate A14

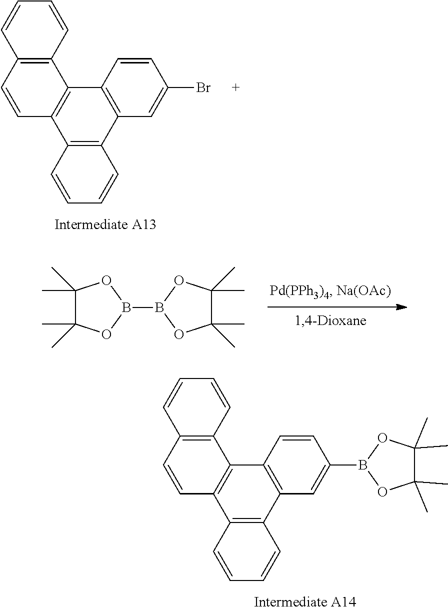

The same synthesis procedure as in Synthesis of Intermediate A5 was used, except that 2 g of Intermediate A13 was used instead of Intermediate A4 to obtain the desired Intermediate A14 (1.5 g, yield=68%).

### Synthesis of Intermediate A15

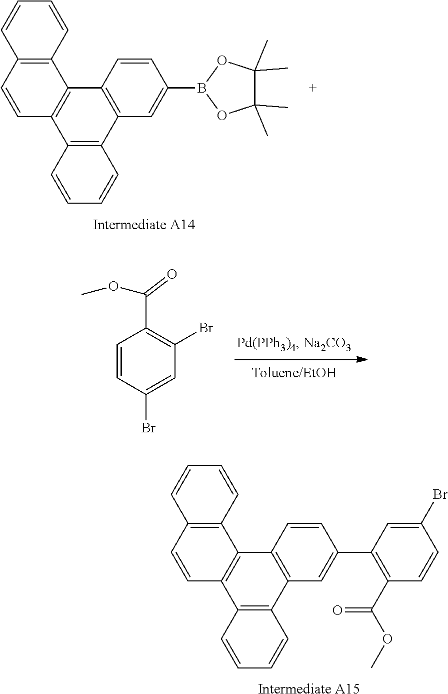

The same synthesis procedure as in Synthesis of Intermediate A7 was used, except that 2 g of Intermediate A14 was used instead of Intermediate A1 to obtain the desired Intermediate A15 (1.6 g, yield=68.3%).

### Synthesis of Intermediate A16

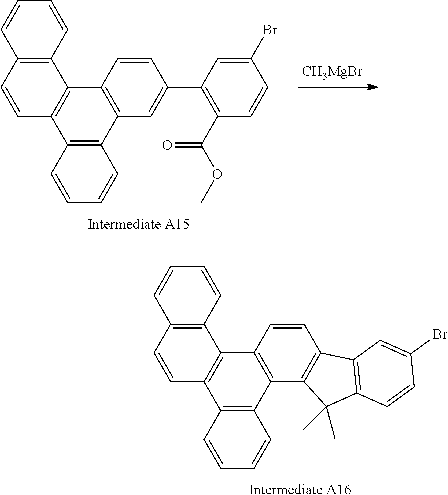

The same synthesis procedure as in Synthesis of Intermediate A8 was used, except that 2.5 g of Intermediate A15 was used instead of Intermediate A7 to obtain the desired Intermediate A16 (1.1 g, yield=42.3%).

### Synthesis of Intermediate A17

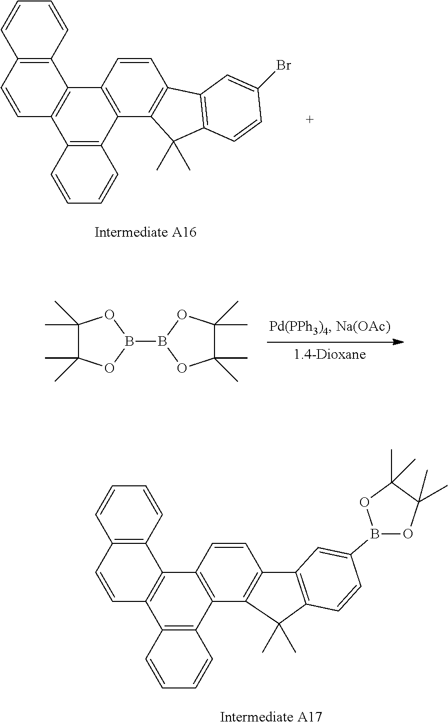

The same synthesis procedure as in Synthesis of Intermediate A9 was used, except that 2 g of Intermediate A16 was used instead of Intermediate A8 to obtain the desired Intermediate A17 (1.5 g, yield=66.7%).

### Synthesis of Intermediate A18

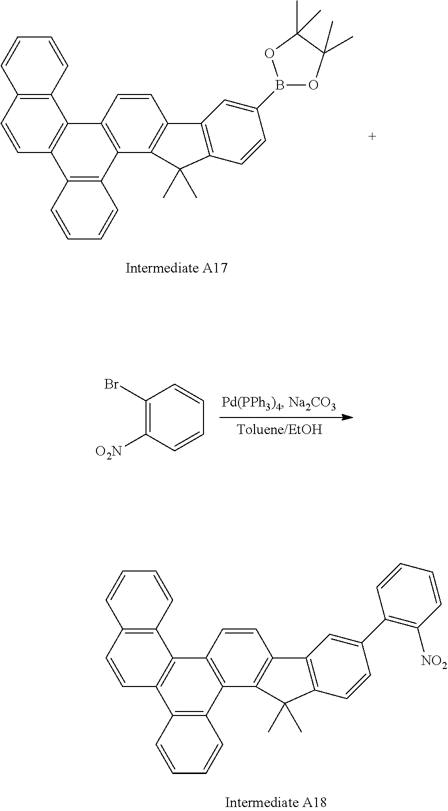 [SMILES: CC1(C)c2ccc(B3OC(C)(C)C(C)(C)O3)cc2-c2ccc3c(c21)c1ccccc1c1ccc2ccccc2c13 ; O=[N+]([O-])c1ccccc1Br ; CC1(C)c2ccc(-c3ccccc3[N+](=O)[O-])cc2-c2ccc3c(c21)c1ccccc1c1ccc2ccccc2c13]

The same synthesis procedure as in Synthesis of Intermediate A10 used, except that 3 g of Intermediate A17 was used instead of Intermediate A9 to obtain the desired Intermediate A18 (1.6 g, yield=56.2%).

### Synthesis of Intermediate A19

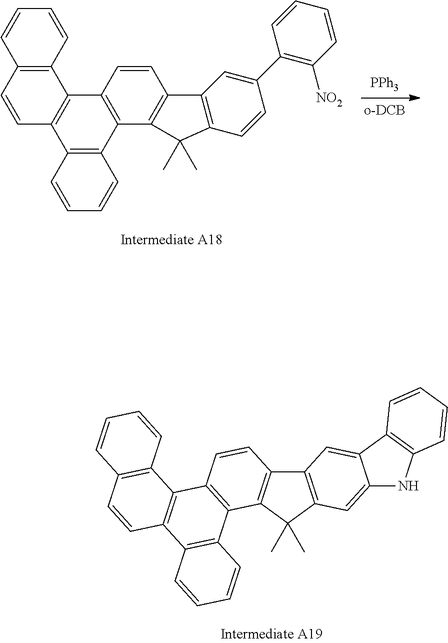 [SMILES: CC1(C)c2ccc(-c3ccccc3[N+](=O)[O-])cc2-c2ccc3c(c21)c1ccccc1c1ccc2ccccc2c13 ; CC1(C)c2cc3[nH]c4ccccc4c3cc2-c2ccc3c(c21)c1ccccc1c1ccc2ccccc2c13 ; "PPh3"]

The same synthesis procedure as in Synthesis of Intermediate A11 used, except that 3 g of Intermediate A18 was used instead of Intermediate A10 to obtain the desired Intermediate A19 (1.2 g, yield=64.2%).

### Synthesis of Compound C4

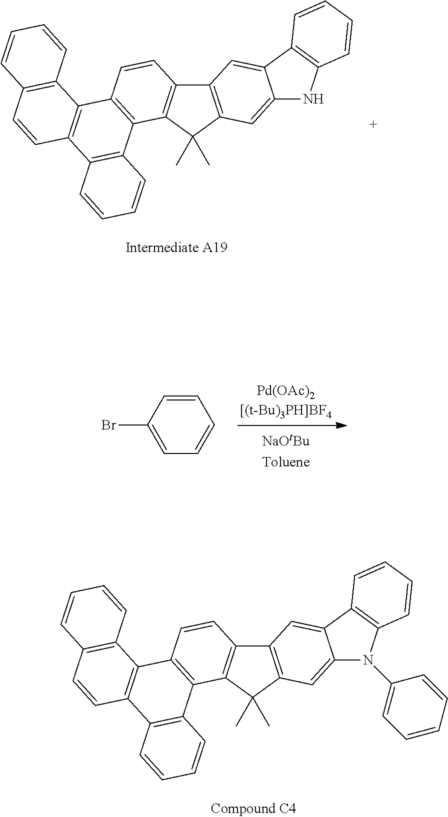 [SMILES: CC1(C)c2cc3[nH]c4ccccc4c3cc2-c2ccc3c(c21)c1ccccc1c1ccc2ccccc2c13 ; Brc1ccccc1 ; CC1(C)c2cc3c(cc2-c2ccc4c(c21)c1ccccc1c1ccc2ccccc2c14)c1ccccc1n3-c1ccccc1]

The same synthesis procedure as in Synthesis of Compound C2 was used, except that 2 g of Intermediate A19 was used instead of Intermediate A11 to obtain the desired Compound C4 (1.2 g, yield=53%).

### Example 4

### Synthesis of Intermediate A20

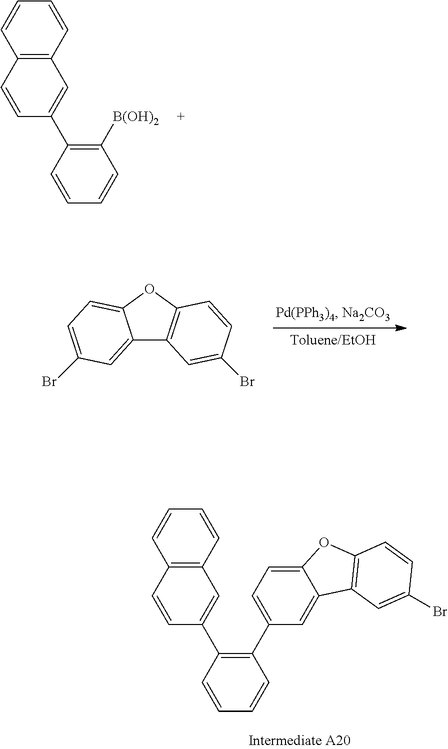 [SMILES: OB(O)c1ccccc1-c1ccc2ccccc2c1 ; Brc1ccc2oc3ccc(Br)cc3c2c1 ; Brc1ccc2oc3ccc(-c4ccccc4-c4ccc5ccccc5c4)cc3c2c1]

The same synthesis procedure as in Synthesis of Intermediate A12 was used, except that 2 g of 2-(naphthalen-2-yl)phenylboronic acid was used instead of 2-phenylnaphthalen-1-ylboronic acid and 1.4 g of 2,8-dibromodibenzo[b,d]furan was used instead of 1,4-dibromobenzene to obtain the desired Intermediate A20 (1.7 g, yield=43.1%).

### Synthesis of Intermediate A21

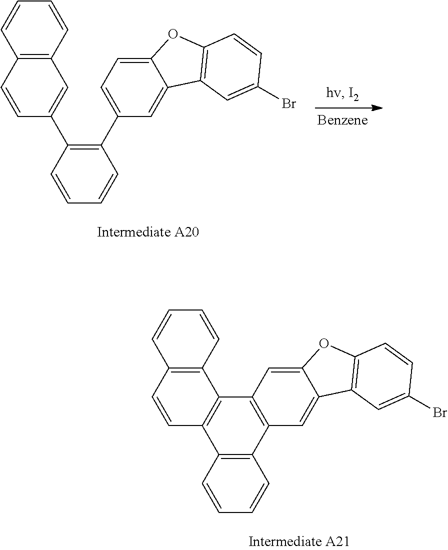 [SMILES: Brc1ccc2oc3ccc(-c4ccccc4-c4ccc5ccccc5c4)cc3c2c1 ; Brc1ccc2oc3cc4c(cc3c2c1)c1ccccc1c1ccc2ccccc2c14]

A mixture of 10 g (22.3 mmol) of Intermediate A20, 0.06 g (0.23 mmol) of Iodine, and 1000 ml of benzene was degassed and placed under nitrogen, and then exposed to UV light for 4 hrs. After the reaction finished, the solvent was removed, and then the residue was recrystallized 3 times to give Intermediate A21 (2.3 g, 23%).

### Synthesis of Intermediate A22

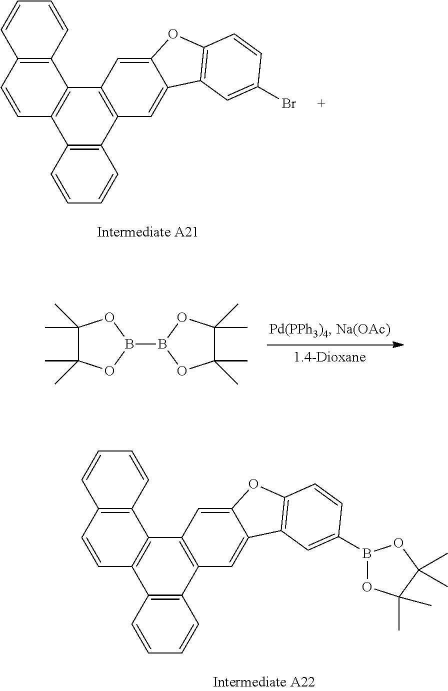

The same synthesis procedure as in Synthesis of Intermediate A1 was used, except that 2 g of Intermediate A21 was used instead of 10-bromobenzo[g]chrysene to obtain the desired Intermediate A22 (1.4 g, yield=63.4%).

### Synthesis of Intermediate A23

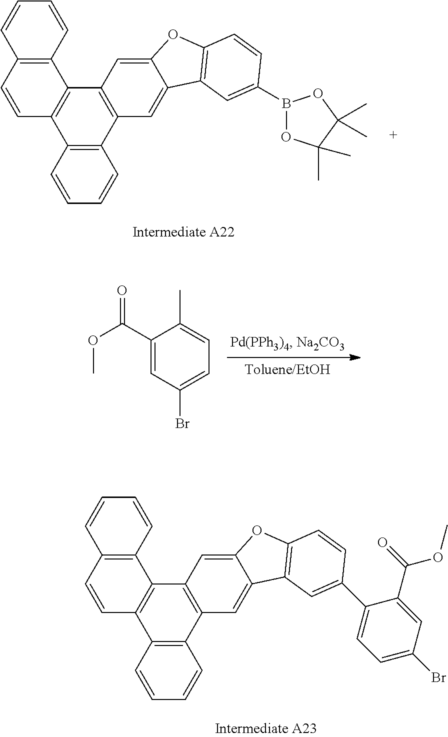 [SMILES: CC1(C)OB(c2ccc3oc4cc5c(cc4c3c2)c2ccccc2c2ccc3ccccc3c25)OC1(C)C ; COC(=O)c1cc(Br)ccc1C ; COC(=O)c1cc(Br)ccc1-c1ccc2oc3cc4c(cc3c2c1)c1ccccc1c1ccc2ccccc2c14]

The same synthesis procedure as in Synthesis of Intermediate A6 was used, except that 3 g of Intermediate A22 was used instead of Intermediate A5 and 2 g of methyl 5-bromo-2-iodobenzoate was used instead of methyl 2-iodobenzoate to obtain the desired Intermediate A23 (2.4 g, yield=78.3%).

### Synthesis of Intermediate A24

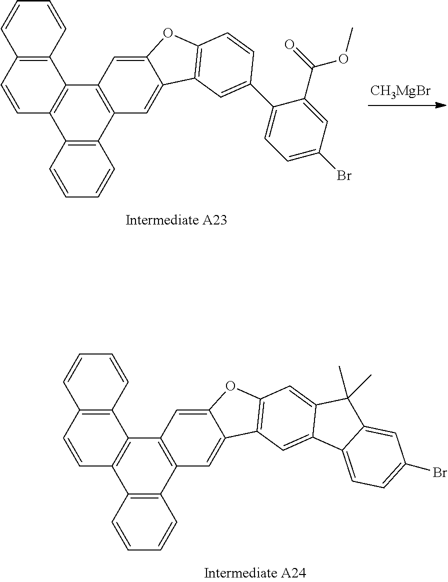

The same synthesis procedure as in Synthesis of Compound C1 was used, except that 2 g of Intermediate A23 was used instead of Intermediate A6 to obtain the desired Intermediate A24 (0.92 g, yield=48.3%).

### Synthesis of Intermediate A25

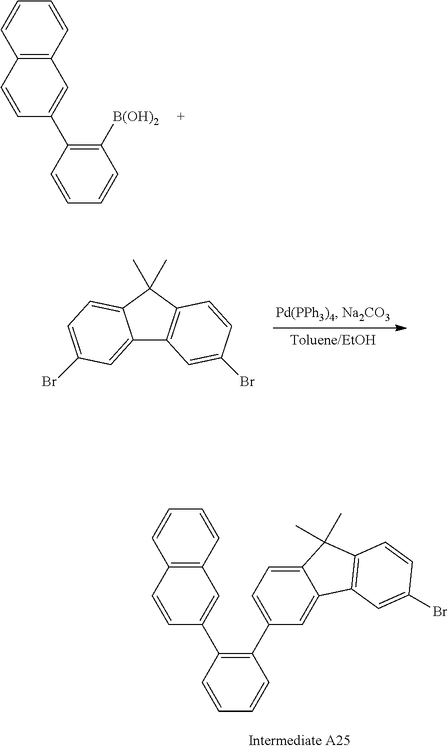 [SMILES: OB(O)c1ccccc1-c1ccc2ccccc2c1 ; CC1(C)c2ccc(Br)cc2-c2cc(Br)ccc21 ; CC1(C)c2ccc(Br)cc2-c2cc(-c3ccccc3-c3ccc4ccccc4c3)ccc21]

The same synthesis procedure as in Synthesis of Intermediate A20 was used, except that 2.8 g of 3,6-dibromo-9,9-dimethyl-9H-fluorene was used instead of 2,8-dibromodibenzo[b,d]furan to obtain the desired Intermediate A25 (1.7 g, yield=45.1%).

### Synthesis of Intermediate A26

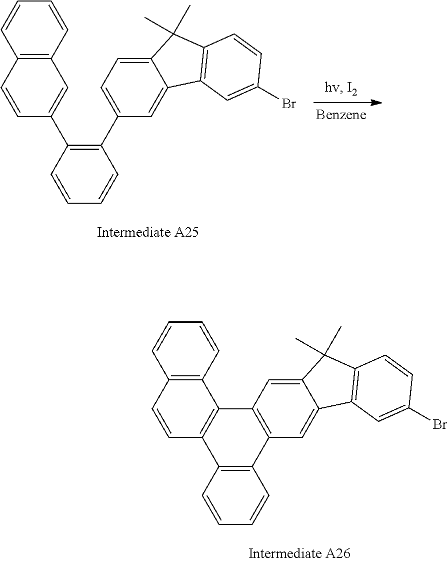 [SMILES: CC1(C)c2ccc(Br)cc2-c2cc(-c3ccccc3-c3ccc4ccccc4c3)ccc21 ; CC1(C)c2ccc(Br)cc2-c2cc3c4ccccc4c4ccc5ccccc5c4c3cc21]

The same synthesis procedure as in Synthesis of Intermediate A21 was used, except that 8 g of Intermediate A25 was used instead of Intermediate A20 to obtain the desired Intermediate A26 (2.3 g, yield=29%).

### Synthesis of Intermediate A27

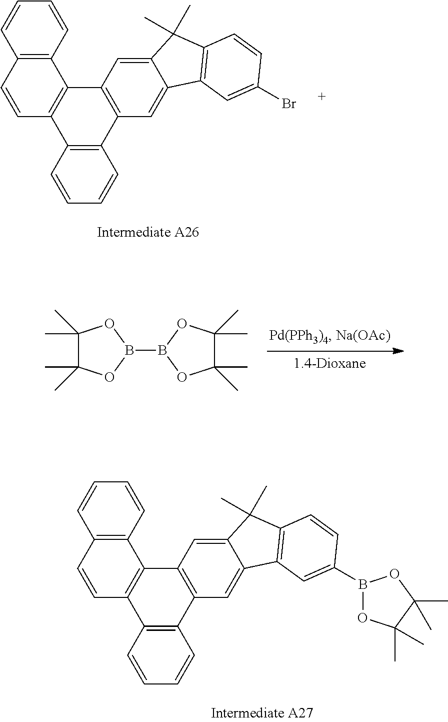

The same synthesis procedure as in Synthesis of Intermediate A1 was used, except that 8 g of Intermediate A26 was used instead of 10-bromobenzo[g]chrysene to obtain the desired Intermediate A27 (6 g, yield=68.1%).

### Synthesis of Intermediate A28

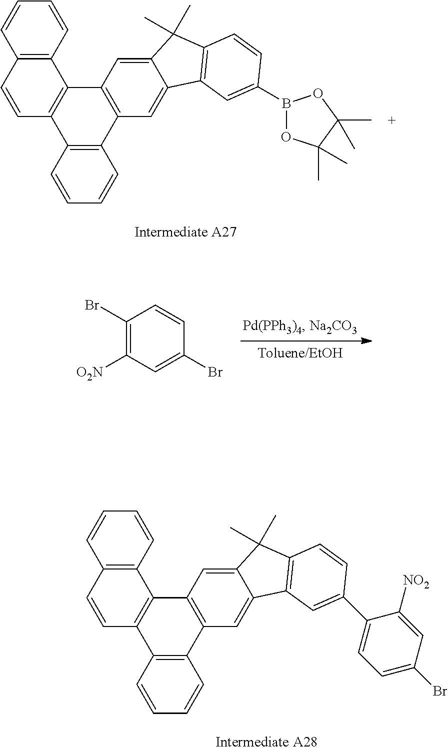 [SMILES: CC1(C)c2ccc(B3OC(C)(C)C(C)(C)O3)cc2-c2cc3c4ccccc4c4ccc5ccccc5c4c3cc21 ; O=[N+]([O-])c1cc(Br)ccc1Br ; CC1(C)c2ccc(-c3ccc(Br)cc3[N+](=O)[O-])cc2-c2cc3c4ccccc4c4ccc5ccccc5c4c3cc21]

The same synthesis procedure as in Synthesis of Intermediate A23 was used, except that 6 g of Intermediate A27 was used instead of Intermediate A22 and 4.1 g of 1,4-dibromo-2-nitrobenzene was used instead of methyl 5-bromo-2-iodobenzoate to obtain the desired Intermediate A28 (3.8 g, yield=54.1%).

### Synthesis of Intermediate A29

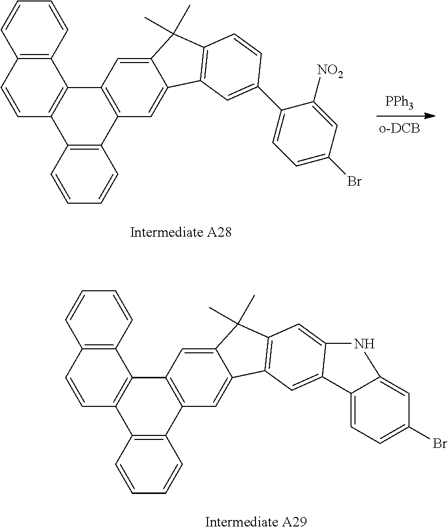 [SMILES: CC1(C)c2ccc(-c3ccc(Br)cc3[N+](=O)[O-])cc2-c2cc3c4ccccc4c4ccc5ccccc5c4c3cc21 ; CC1(C)c2cc3[nH]c4cc(Br)ccc4c3cc2-c2cc3c4ccccc4c4ccc5ccccc5c4c3cc21 ; "PPh3"]

The same synthesis procedure as in Synthesis of Intermediate A3 was used, except that 5 g of Intermediate A28 was used instead of Intermediate A2 to obtain the desired Intermediate A29 (2.3 g, yield=50%).

### Synthesis of Intermediate A30

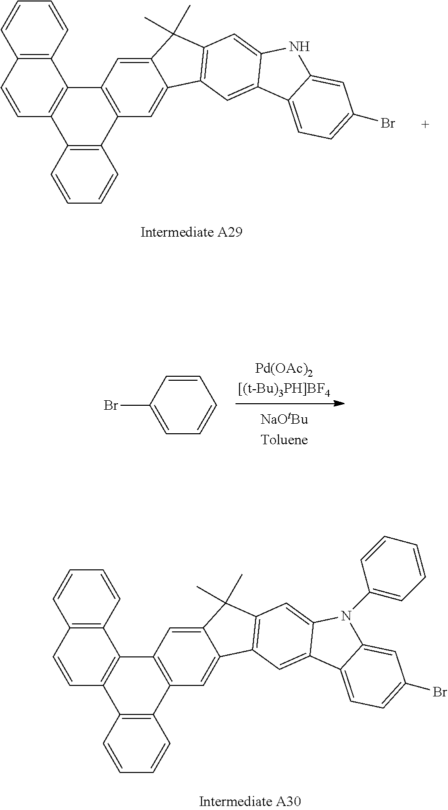 [SMILES: CC1(C)c2cc3[nH]c4cc(Br)ccc4c3cc2-c2cc3c4ccccc4c4ccc5ccccc5c4c3cc21 ; Brc1ccccc1 ; CC1(C)c2cc3c(cc2-c2cc4c5ccc(Br)cc5n(-c5ccccc5)c4cc21)c1ccccc1c1ccc2ccccc2c13]

The same synthesis procedure as in Synthesis of Compound C2 was used, except that 3 g of Intermediate A29 was used instead of Intermediate A11 to obtain the desired Intermediate A30 (1.5 g, yield=43.3%).

### Synthesis of Intermediate A31

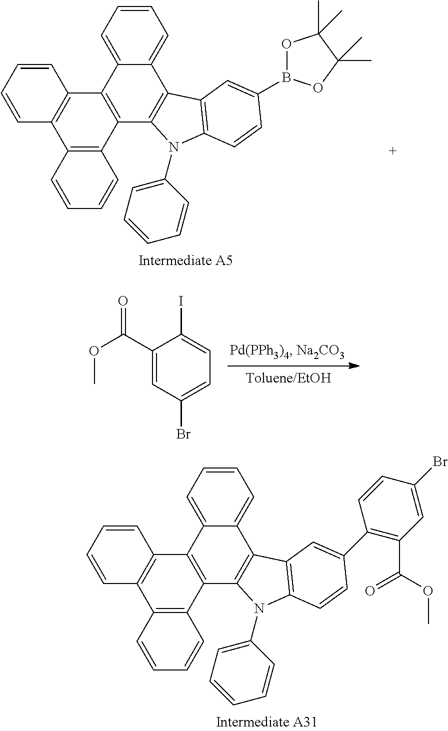

The same synthesis procedure as in Synthesis of Intermediate A6 was used, except that 6 g of methyl 5-bromo-2-iodobenzoate was used instead of methyl 2-iodobenzoate to obtain the desired Intermediate A31 (7.8 g, yield=67.8%).

### Synthesis of Intermediate A32

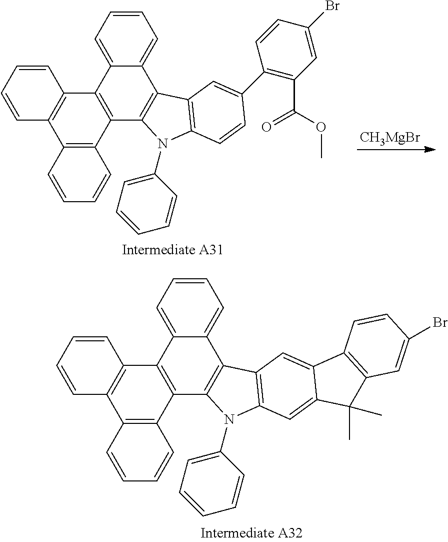

The same synthesis procedure as in Synthesis of Compound C1 was used, except that 5 g of Intermediate A31 was used instead of Intermediate A6 to obtain the desired Intermediate A32 (2.3 g, yield=47.3%).

### Synthesis of Intermediate A33

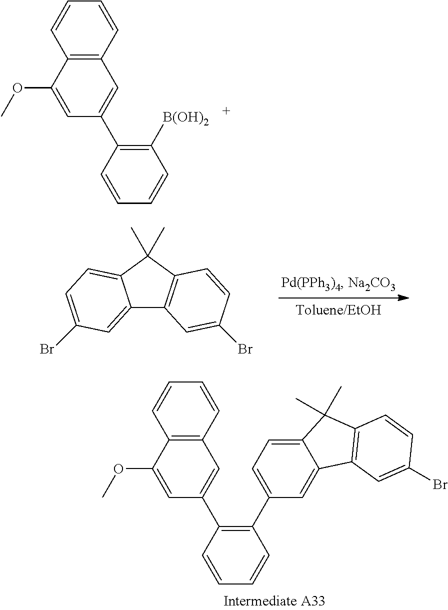

The same synthesis procedure as in Synthesis of Intermediate A25 was used, except that 3 g of 2-(4-methoxynaphthalen-2-yl)phenylboronic acid was used instead of 2-(naphthalen-2-yl)phenylboronic acid to obtain the desired Intermediate A33 (2.3 g, yield=43.1%).

### Synthesis of Intermediate A34

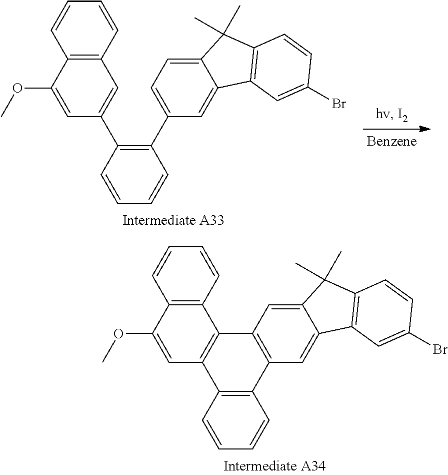 [SMILES: COc1cc(-c2ccccc2-c2ccc3c(c2)-c2cc(Br)ccc2C3(C)C)cc2ccccc12 ; COc1cc2c3ccccc3c3cc4c(cc3c2c2ccccc12)C(C)(C)c1ccc(Br)cc1-4]

The same synthesis procedure as in Synthesis of Intermediate A26 was used, except that 8 g of Intermediate A33 was used instead of Intermediate A24 to obtain the desired Intermediate A34 (1.8 g, yield=30%).

### Synthesis of Intermediate A35

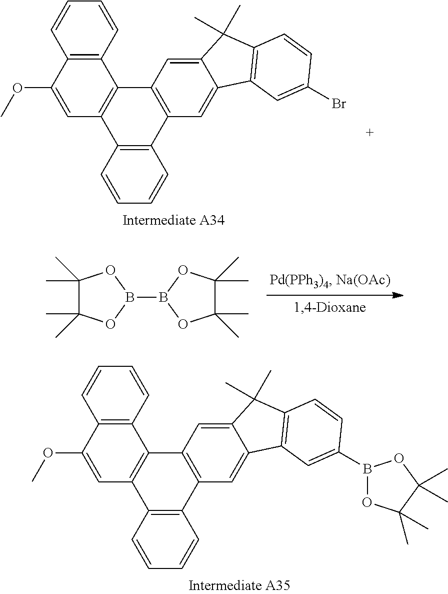

The same synthesis procedure as in Synthesis of Intermediate A27 was used, except that 6 g of Intermediate A34 was used instead of Intermediate A26 to obtain the desired Intermediate A35 (4.5 g, yield=69.2%).

### Synthesis of Intermediate A36

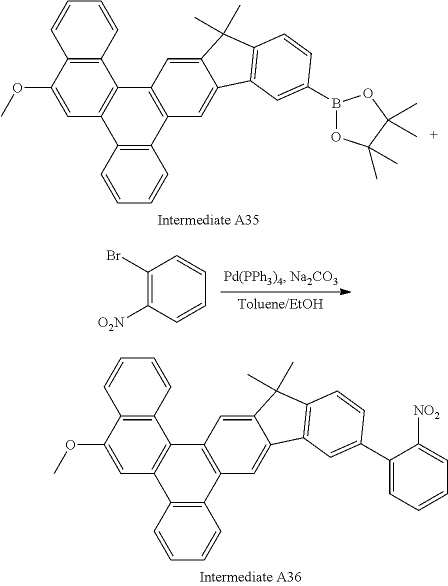

The same synthesis procedure as in Synthesis of Intermediate A28 was used, except that 6 g of Intermediate A35 was used instead of Intermediate A27 and 2.2 g of 1-bromo-2-nitrobenzene was used instead of 1,4-dibromo-2-nitrobenzene to obtain the desired Intermediate A36 to obtain the desired Intermediate A36 (3.8 g, yield=63.5%).

### Synthesis of Intermediate A37

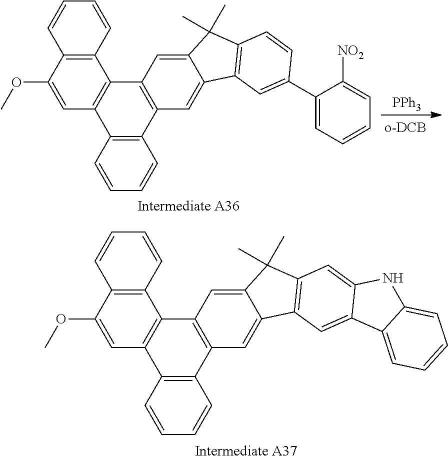

The same synthesis procedure as in Synthesis of Intermediate A29 was used, except that 5 g of Intermediate A36 was used instead of Intermediate A28 to obtain the desired Intermediate A37 (2.3 g, yield=48.3%).

### Synthesis of Intermediate A38

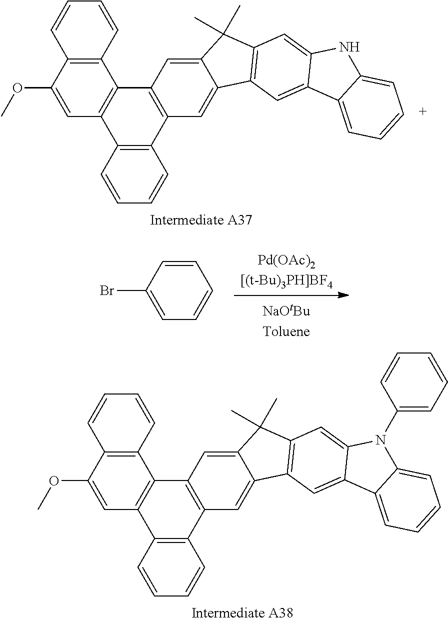 [SMILES: COc1cc2c3ccccc3c3cc4c(cc3c2c2ccccc12)C(C)(C)c1cc2[nH]c3ccccc3c2cc1-4 ; Brc1ccccc1 ; COc1cc2c3ccccc3c3cc4c(cc3c2c2ccccc12)C(C)(C)c1cc2c(cc1-4)c1ccccc1n2-c1ccccc1]

The same synthesis procedure as in Synthesis of Intermediate A30 was used, except that 4 g of Intermediate A37 was used instead of Intermediate A29 to obtain the desired Intermediate A38 (2.5 g, yield=55.3%).

### Synthesis of Intermediate A39

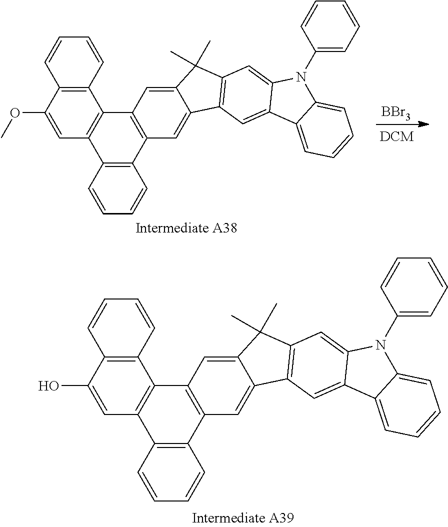

A mixture of 3 g (5.08 mmol) of Intermediate A38 and 60 ml of dichloromethane was placed into the reactor under nitrogen. Boron tribromide (1 eq.) was added thereto and then stirred for 2 hrs until the reaction finished. The reaction mixture was extracted with dichloromethane and water, and then dried with anhydrous MgSO₄. The solvent was removed to give Intermediate A39 (2.6 g, yield=89.1%).

### Synthesis of Intermediate A40

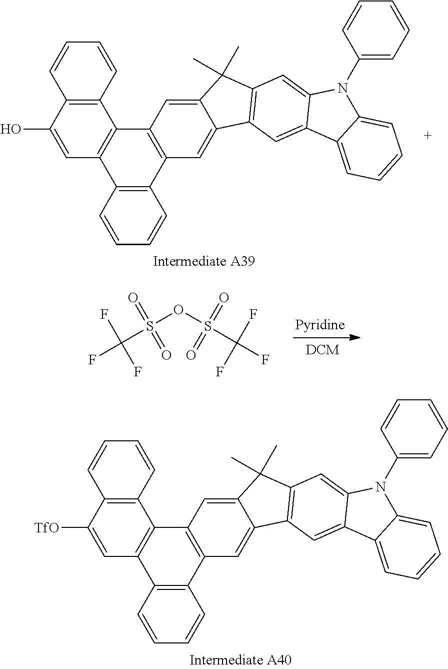 [SMILES: CC1(C)c2cc3c(cc2-c2cc4c5ccccc5n(-c5ccccc5)c4cc21)c1ccccc1c1cc(O)c2ccccc2c31 ; O=S(=O)(OS(=O)(=O)C(F)(F)F)C(F)(F)F ; CC1(C)c2cc3c(cc2-c2cc4c5ccccc5n(-c5ccccc5)c4cc21)c1ccccc1c1cc(OS(=O)(=O)C(F)(F)F)c2ccccc2c31]

A mixture of 2.6 g (4.51 mmol) of Intermediate A39 and 60 ml of dichloromethane was placed into the reactor under nitrogen. Pyridine (1.5 eq.) and trifluoromethanesulfonic anhydride (1.7 eq) was added thereto and then stirred for 12 hrs until the reaction finished. The reaction mixture was extracted with dichloromethane and water, and then dried with anhydrous MgSO₄. The solvent was removed to give Intermediate A40 (2.9 g, yield=92.8%).

### Synthesis of Intermediate A41

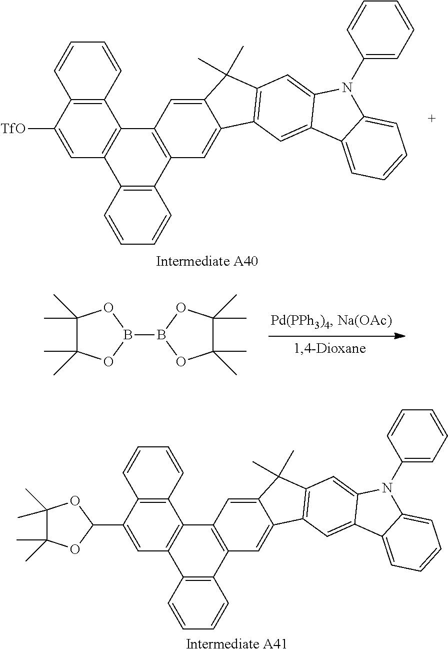

The same synthesis procedure as in Synthesis of Intermediate A35 was used, except that 5 g of Intermediate A40 was used instead of Intermediate A34 to obtain the desired Intermediate A41 (3.4 g, yield=71.2%).

### Synthesis of Intermediate A42

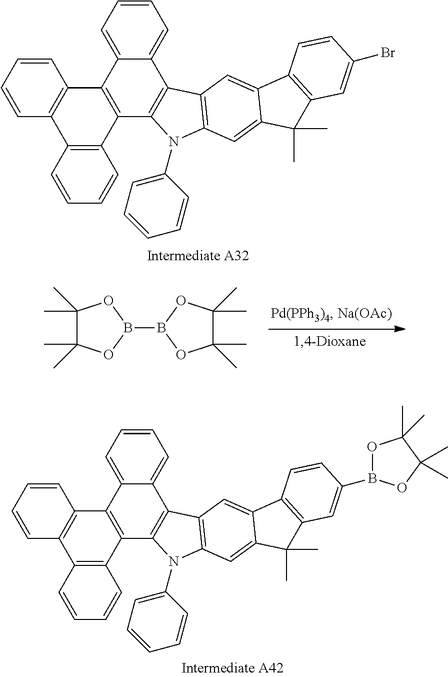

The same synthesis procedure as in Synthesis of Intermediate A41 was used, except that 4 g of Intermediate A32 was used instead of Intermediate A40 to obtain the desired Intermediate A42 (2.8 g, yield=65.2%).

### Synthesis of Intermediate A43

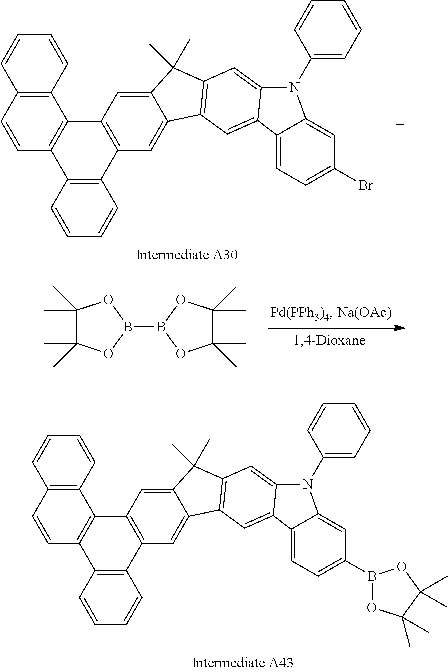

The same synthesis procedure as in Synthesis of Intermediate A41 was used, except that 4 g of Intermediate A30 was used instead of Intermediate A40 to obtain the desired Intermediate A43 (3.1 g, yield=72.3%).

### Synthesis of Compound C71

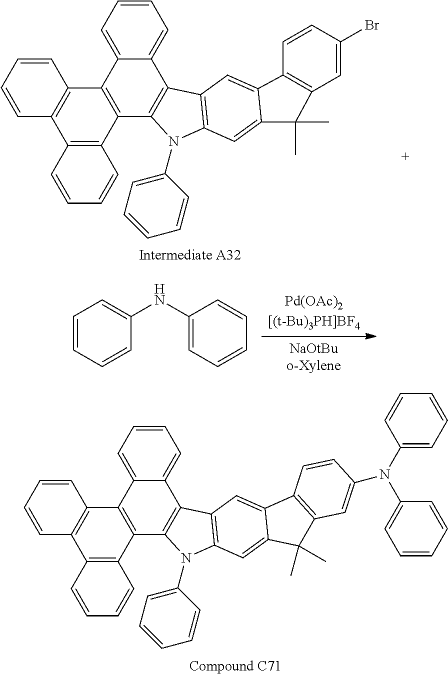 [SMILES: CC1(C)c2cc(Br)ccc2-c2cc3c4c5ccccc5c5c6ccccc6c6ccccc6c5c4n(-c4ccccc4)c3cc21 ; c1ccc(Nc2ccccc2)cc1 ; CC1(C)c2cc(N(c3ccccc3)c3ccccc3)ccc2-c2cc3c4c5ccccc5c5c6ccccc6c6ccccc6c5c4n(-c4ccccc4)c3cc21]

A mixture of 3.0 g (4.7 mmol) of Intermediate A32, 0.9 g (5.6 mmol) of diphenylamine, 0.04 g (0.18 mmol) of Pd(OAc)₂, 0.1 g (0.47 mmol) of tri-tert-butylphosphonium tetrafluoroborate, 1.3 g (14 mmol) of sodium tert-butoxide, and 60 ml of o-xylene was degassed and placed under nitrogen gas, and then heated at 150° C. for 8 hrs. After the reaction finished, the mixture was allowed to cool to room temperature. Subsequently, the organic layer was extracted with dichloromethane and water, and then dried with anhydrous MgSO₄. After the solvent was removed, the residue was purified by column chromatography on silica to give compound C71 (2.4 g, 72.3%). MS(m/z, FAB+): 726.3.

### Example 5

### Synthesis of Compound C72

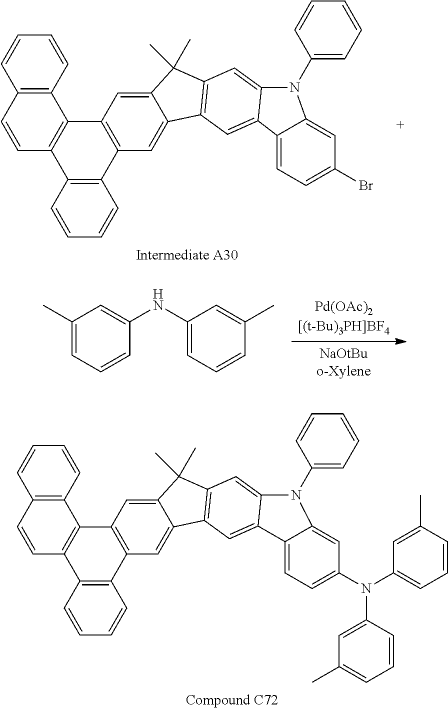

The same synthesis procedure as in Synthesis of compound C71 was used, except that 3 g of Intermediate A30 was used instead of Intermediate A32 and dim-tolylamine was used instead of diphenylamine to obtain the desired compound C72 (2.4 g, 68.3%). MS(m/z, FAB+): 754.4.

### Example 6

### Synthesis of Compound C80

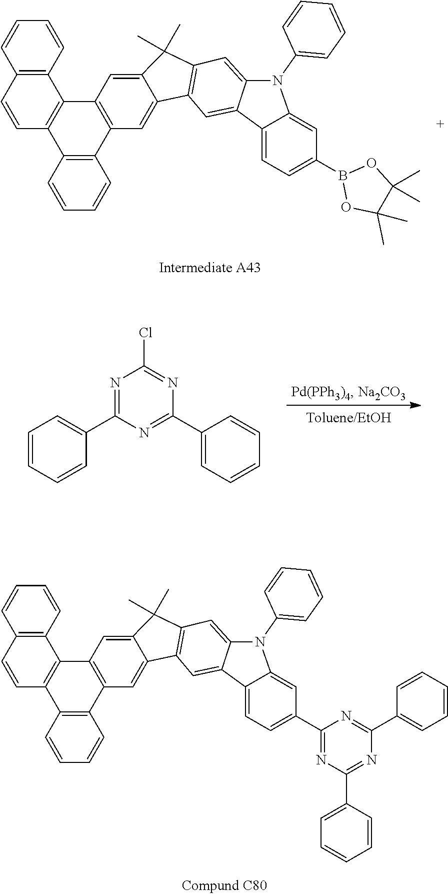 [SMILES: CC1(C)c2cc3c(cc2-c2cc4c5ccc(B6OC(C)(C)C(C)(C)O6)cc5n(-c5ccccc5)c4cc21)c1ccccc1c1ccc2ccccc2c13 ; Clc1nc(-c2ccccc2)nc(-c2ccccc2)n1 ; CC1(C)c2cc3c(cc2-c2cc4c5ccc(-c6nc(-c7ccccc7)nc(-c7ccccc7)n6)cc5n(-c5ccccc5)c4cc21)c1ccccc1c1ccc2ccccc2c13]

A mixture of 1 g (1.5 mmol) of Intermediate A43, 1.1 g (1.8 mmol) of 2-chloro-4,6-diphenyl-1,3,5-triazine, 0.01 g (0.03 mmol) of Pd(PPh₃)₄, 4 ml of 2M Na₂CO_{3(aq)}, 10 ml of EtOH, and 30 ml of toluene was degassed and placed under nitrogen, and then heated at 100° C. for 12 hrs. After the reaction finished, the mixture was allowed to cool to room temperature. Subsequently, the organic layer was extracted with dichloromethane and water, and then dried with anhydrous MgSO₄. After the solvent was removed, the residue was purified by column chromatography on silica to give compound C80 (0.6 g, 53%). MS(m/z, FAB+): 790.31.

### Example 7

### Synthesis of Compound C77

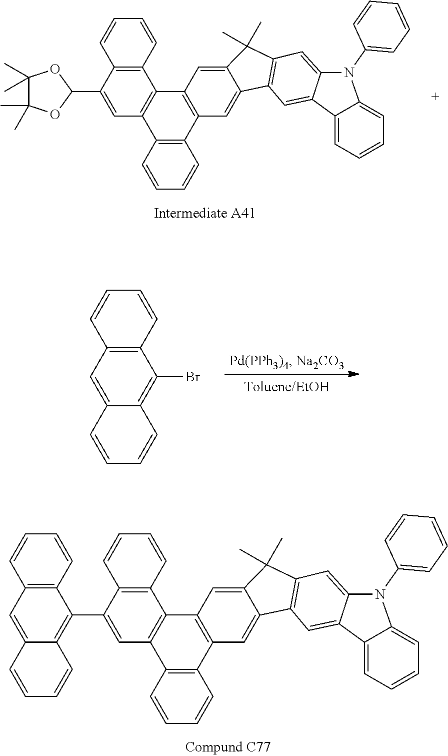 [SMILES: CC1(C)c2cc3c(cc2-c2cc4c5ccccc5n(-c5ccccc5)c4cc21)c1ccccc1c1cc(C2OC(C)(C)C(C)(C)O2)c2ccccc2c31 ; Brc1c2ccccc2cc2ccccc12 ; CC1(C)c2cc3c(cc2-c2cc4c5ccccc5n(-c5ccccc5)c4cc21)c1ccccc1c1cc(-c2c4ccccc4cc4ccccc24)c2ccccc2c31]

The same synthesis procedure as in Synthesis of compound C80 was used, except that 3 g of Intermediate A41 was used instead of Intermediate A43 and 9-bromoanthracene was used instead of 2-chloro-4,6-diphenyl-1,3,5-triazine to obtain the desired compound C77 (2.2 g, 68.3%). MS(m/z, FAB+): 735.28.

### Example 8

### Synthesis of Compound C78

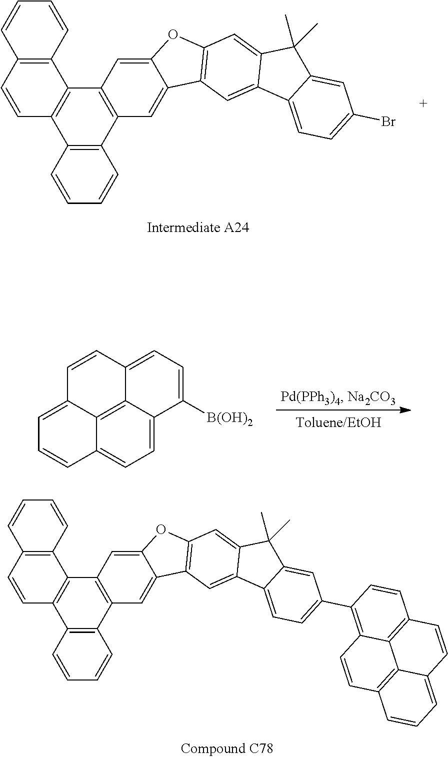

The same synthesis procedure as in Synthesis of Intermediate A20 was used, except that 3 g of Intermediate A24 was used instead of 2,8-dibromodibenzo[b,d]furan and 1.4 g of pyren-1-ylboronic acid was used instead of 2-(naphthalen-2-yl)phenylboronic acid to obtain the desired Compound C78 (1.8 g, yield=50.3%).

### Example 9

### Synthesis of Compound C79

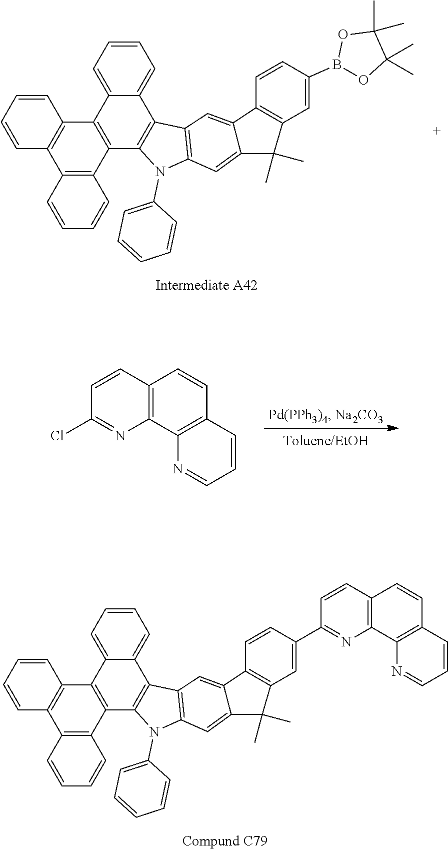 [SMILES: CC1(C)c2cc(B3OC(C)(C)C(C)(C)O3)ccc2-c2cc3c4c5ccccc5c5c6ccccc6c6ccccc6c5c4n(-c4ccccc4)c3cc21 ; Clc1ccc2ccc3cccnc3c2n1 ; CC1(C)c2cc(-c3ccc4ccc5cccnc5c4n3)ccc2-c2cc3c4c5ccccc5c5c6ccccc6c6ccccc6c5c4n(-c4ccccc4)c3cc21]

The same synthesis procedure as in Synthesis of Intermediate A36 was used, except that 3 g of Intermediate A42 was used instead of Intermediate A35 and 1.1 g of 2-chloro-1,10-phenanthroline was used instead of 1-bromo-2-nitrobenzene to obtain the desired Compound C79 (2.1 g, yield=66.2%). MS(m/z, FAB+): 737.6.

### Example 10

### Synthesis of Compound C85

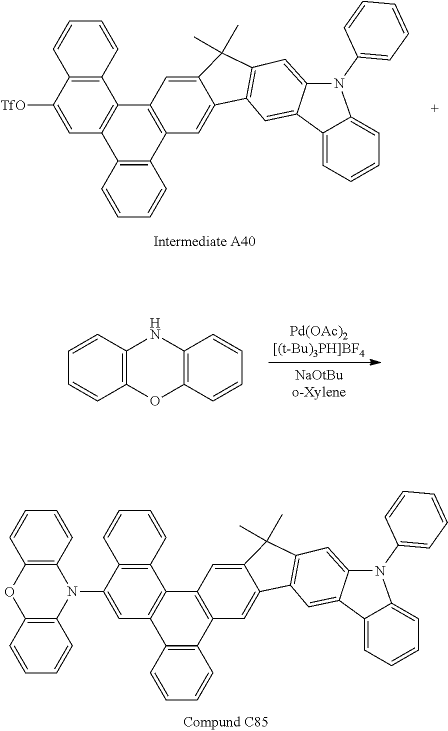 [SMILES: CC1(C)c2cc3c(cc2-c2cc4c5ccccc5n(-c5ccccc5)c4cc21)c1ccccc1c1cc(OS(=O)(=O)C(F)(F)F)c2ccccc2c31 ; c1ccc2c(c1)Nc1ccccc1O2 ; CC1(C)c2cc3c(cc2-c2cc4c5ccccc5n(-c5ccccc5)c4cc21)c1ccccc1c1cc(N2c4ccccc4Oc4ccccc42)c2ccccc2c31]

The same synthesis procedure as in Synthesis of compound C71 was used, except that 3 g of Intermediate A40 was used instead of Intermediate A32 and 10H-phenoxazine was used instead of diphenylamine to obtain the desired compound C85 (2.4 g, 68.3%). MS(m/z, FAB+): 740.29.

### Example 11

### Synthesis of Compound C89

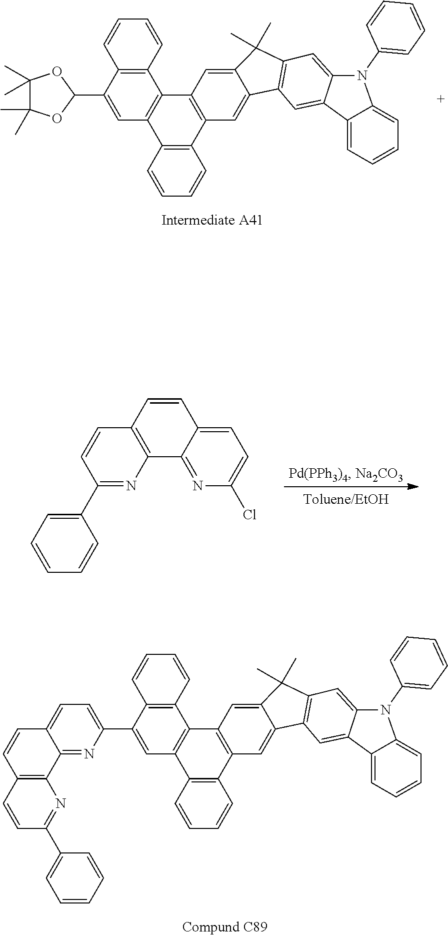 [SMILES: CC1(C)c2cc3c(cc2-c2cc4c5ccccc5n(-c5ccccc5)c4cc21)c1ccccc1c1cc(C2OC(C)(C)C(C)(C)O2)c2ccccc2c31 ; Clc1ccc2ccc3ccc(-c4ccccc4)nc3c2n1 ; CC1(C)c2cc3c(cc2-c2cc4c5ccccc5n(-c5ccccc5)c4cc21)c1ccccc1c1cc(-c2ccc4ccc5ccc(-c6ccccc6)nc5c4n2)c2ccccc2c31]

The same synthesis procedure as in Synthesis of compound C80 was used, except that 3 g of Intermediate A41 was used instead of Intermediate A43 and 2-chloro-9-phenyl-1,10-phenanthroline was used instead of 2-chloro-4,6-diphenyl-1,3,5-triazine to obtain the desired compound C89 (2.3 g, 64.3%). MS(m/z, FAB+): 813.32.

### Example 12

### Synthesis of Intermediate A44

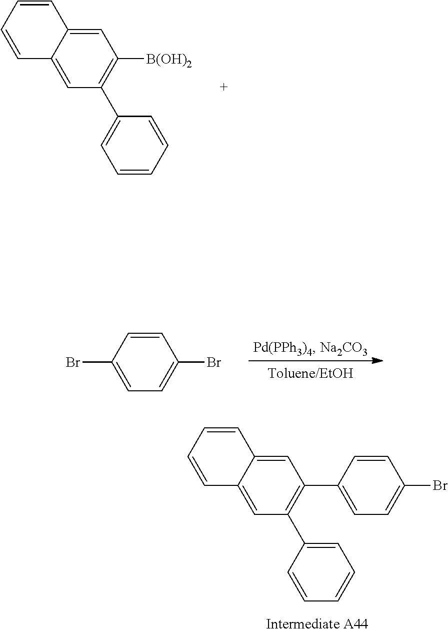 [SMILES: OB(O)c1cc2ccccc2cc1-c1ccccc1 ; Brc1ccc(Br)cc1 ; Brc1ccc(-c2cc3ccccc3cc2-c2ccccc2)cc1]

The same synthesis procedure as in Synthesis of Intermediate A2 was used, except that 3 g of 3-phenylnaphthalen-2-ylboronic acid was used instead of Intermediate A1 and 2.8 g of 1,4-dibromobenzene was used instead of 2,5-dibromonitrobenzene to obtain the desired Intermediate A44 (2.1 g, yield=50%).

### Synthesis of Intermediate A45

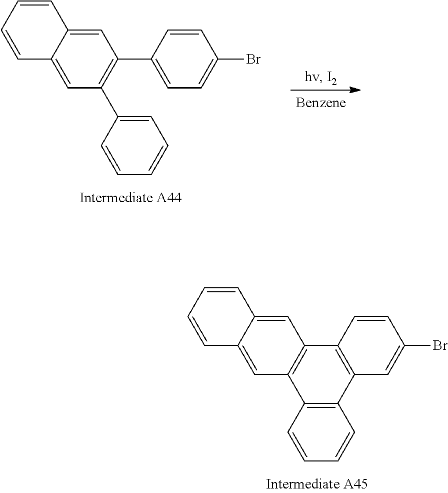 [SMILES: Brc1ccc(-c2cc3ccccc3cc2-c2ccccc2)cc1 ; Brc1ccc2c(c1)c1ccccc1c1cc3ccccc3cc21]

A mixture of 10 g (27.8 mmol) of Intermediate A44, 0.07 g (0.28 mmol) of Iodine, and 1000 ml of benzene was degassed and placed under nitrogen, and then exposed to UV light for 4 hrs. After the reaction finished, the solvent was removed, and then the residue was recrystallized 3 times to give Intermediate A45 (2.3 g, 23%).

### Synthesis of Intermediate A46

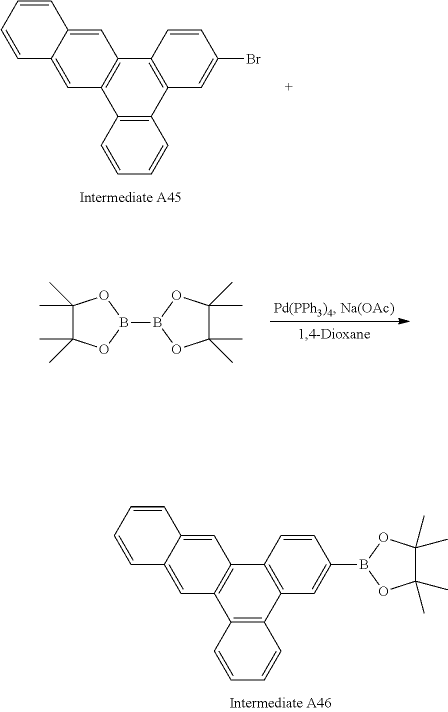

The same synthesis procedure as in Synthesis of Intermediate A5 was used, except that 2 g of Intermediate A45 was used instead of Intermediate A4 to obtain the desired Intermediate A46 (1.5 g, yield=68%).

### Synthesis of Intermediate A47

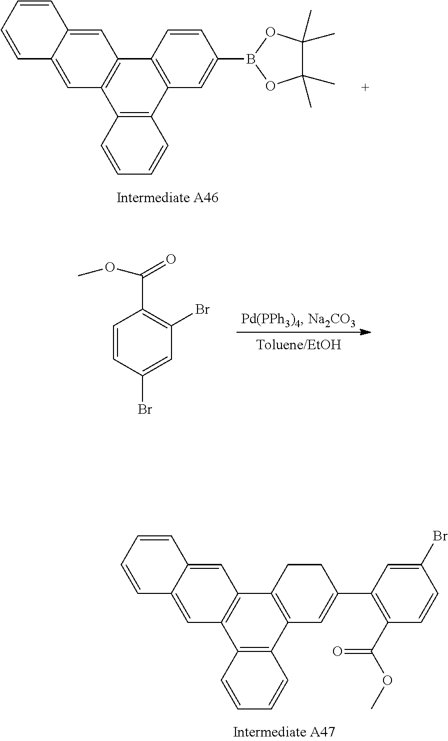

The same synthesis procedure as in Synthesis of Intermediate A7 was used, except that 2 g of Intermediate A46 was used instead of Intermediate A1 to obtain the desired Intermediate A47 (1.9 g, yield=81.1%).

### Synthesis of Intermediate A48

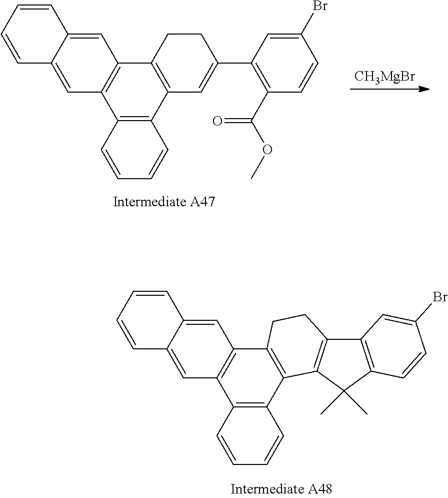

The same synthesis procedure as in Synthesis of Intermediate A8 was used, except that 3 g of Intermediate A47 was used instead of Intermediate A7 to obtain the desired Intermediate A48 (1.1 g, yield=61.3%).

### Synthesis of Intermediate A49

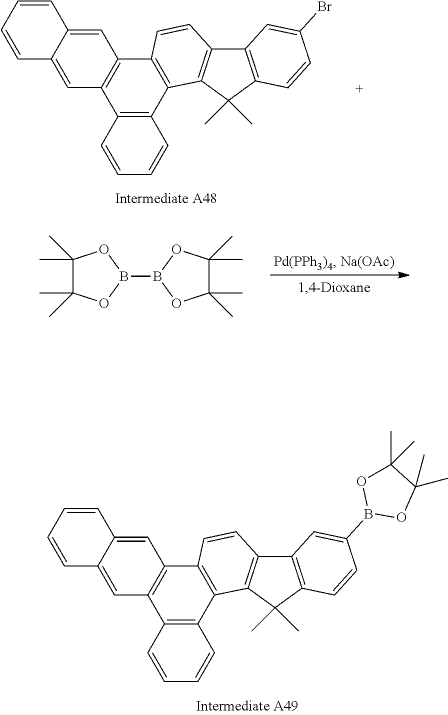

The same synthesis procedure as in Synthesis of Intermediate A9 was used, except that 3 g of Intermediate A48 was used instead of Intermediate A8 to obtain the desired Intermediate A49 (2.3 g, yield=72.1%).

### Synthesis of Intermediate A50

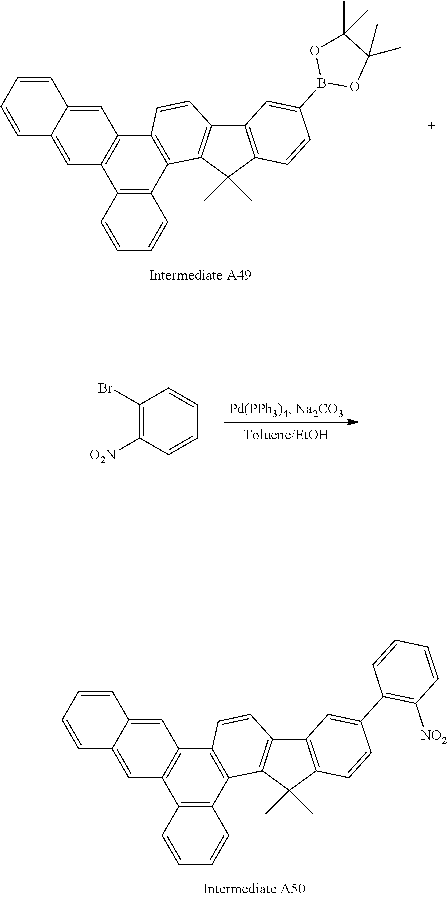 [SMILES: CC1(C)c2ccc(B3OC(C)(C)C(C)(C)O3)cc2-c2ccc3c4cc5ccccc5cc4c4ccccc4c3c21 ; O=[N+]([O-])c1ccccc1Br ; CC1(C)c2ccc(-c3ccccc3[N+](=O)[O-])cc2-c2ccc3c4cc5ccccc5cc4c4ccccc4c3c21]

The same synthesis procedure as in Synthesis of Intermediate A2 was used, except that 3 g of Intermediate A49 was used instead of Intermediate A1 and 1.1 g of 1-bromo-2-nitrobenzene was used instead of 2,4-dibromonitrobenzene to obtain the desired Intermediate A50 (2.5 g, yield=67.3%).

### Synthesis of Intermediate A51

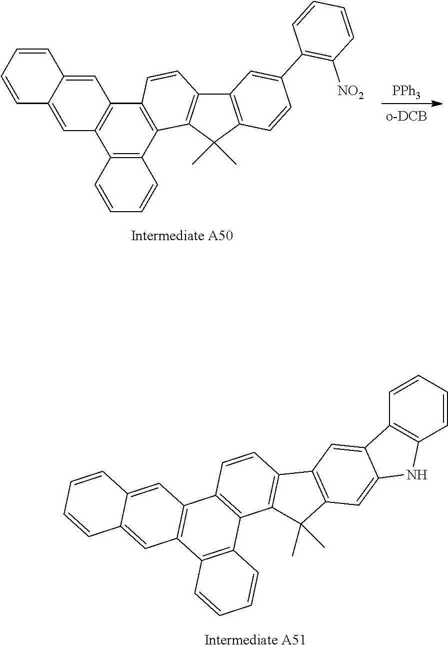 [SMILES: CC1(C)c2ccc(-c3ccccc3[N+](=O)[O-])cc2-c2ccc3c4cc5ccccc5cc4c4ccccc4c3c21 ; CC1(C)c2cc3[nH]c4ccccc4c3cc2-c2ccc3c4cc5ccccc5cc4c4ccccc4c3c21 ; "PPh3"]

The same synthesis procedure as in Synthesis of Intermediate A3 was used, except that 3 g of Intermediate A50 was used instead of Intermediate A2 to obtain the desired Intermediate A51 (1.9 g, yield=67.6%).

### Synthesis of Compound C6

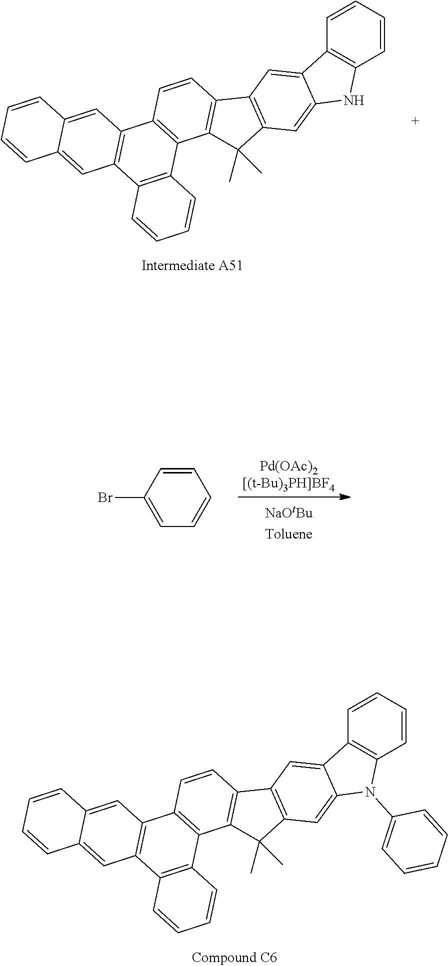 [SMILES: CC1(C)c2cc3[nH]c4ccccc4c3cc2-c2ccc3c4cc5ccccc5cc4c4ccccc4c3c21 ; Brc1ccccc1 ; CC1(C)c2cc3c(cc2-c2ccc4c5cc6ccccc6cc5c5ccccc5c4c21)c1ccccc1n3-c1ccccc1]

The same synthesis procedure as in Synthesis of Intermediate A4 was used, except that 3 g of Intermediate A51 was used instead of Intermediate A3 to obtain the desired Compound C6 (1.2 g, yield=53.1%).

### Example 13

### Synthesis of Intermediate A52

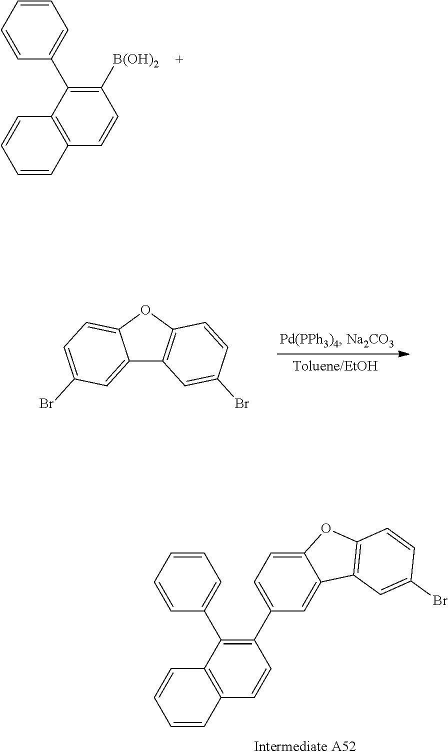 [SMILES: OB(O)c1ccc2ccccc2c1-c1ccccc1 ; Brc1ccc2oc3ccc(Br)cc3c2c1 ; Brc1ccc2oc3ccc(-c4ccc5ccccc5c4-c4ccccc4)cc3c2c1]

The same synthesis procedure as in Synthesis of Intermediate A12 was used, except that 2 g of (1-phenylnaphthalen-2-yl)boronic acid was used instead of 2-phenylnaphthalen-1-ylboronic acid and 1.4 g of 2,8-dibromodibenzo[b,d]furan was used instead of 1,4-dibromobenzene to obtain the desired Intermediate A52 (1.8 g, yield=45.9%).

### Synthesis of Intermediate A53

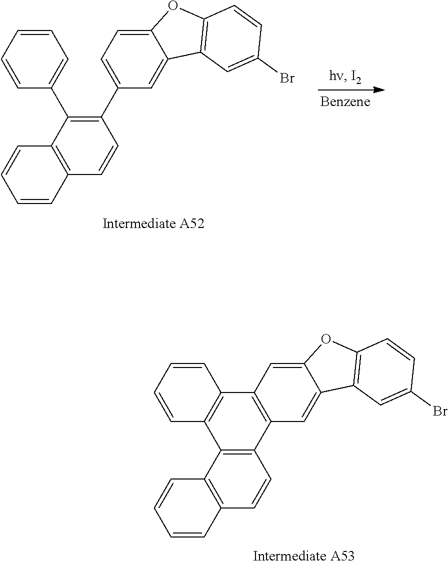 [SMILES: Brc1ccc2oc3ccc(-c4ccc5ccccc5c4-c4ccccc4)cc3c2c1 ; Brc1ccc2oc3cc4c5ccccc5c5c6ccccc6ccc5c4cc3c2c1]

A mixture of 10 g (22.3 mmol) of Intermediate A52, 0.06 g (0.23 mmol) of Iodine, and 1000 ml of benzene was degassed and placed under nitrogen, and then exposed to UV light for 4 hrs. After the reaction finished, the solvent was removed, and then the residue was recrystallized 3 times to give Intermediate A53 (2.1 g, 21%).

### Synthesis of Intermediate A54

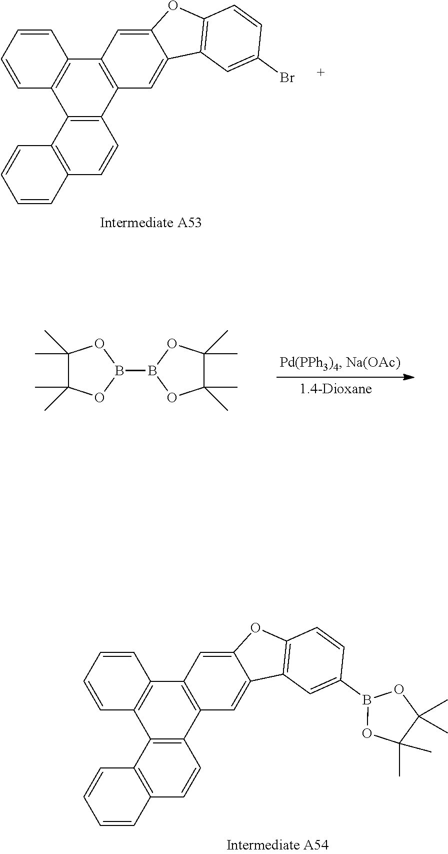

The same synthesis procedure as in Synthesis of Intermediate A17 was used, except that 2 g of Intermediate A53 was used instead of Intermediate A16 to obtain the desired Intermediate A54 (1.3 g, yield=59.1%).

### Synthesis of Intermediate A55

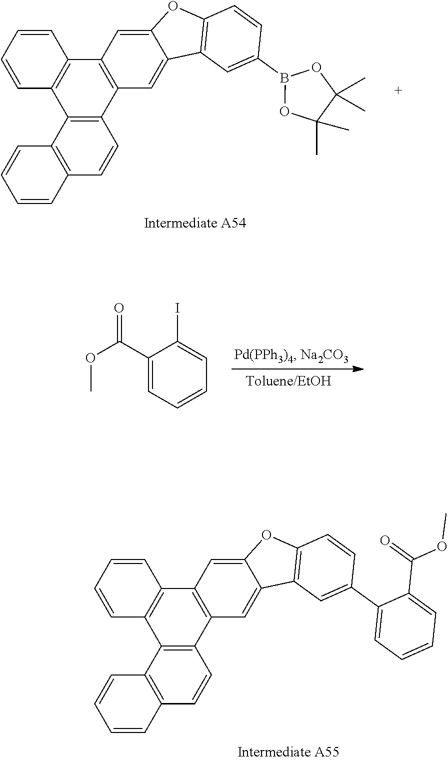 [SMILES: CC1(C)OB(c2ccc3oc4cc5c6ccccc6c6c7ccccc7ccc6c5cc4c3c2)OC1(C)C ; COC(=O)c1ccccc1I ; COC(=O)c1ccccc1-c1ccc2oc3cc4c5ccccc5c5c6ccccc6ccc5c4cc3c2c1]

The same synthesis procedure as in Synthesis of Intermediate A6 was used, except that 3 g of Intermediate A54 was used instead of Intermediate A5 to obtain the desired Intermediate A55 (2.5 g, yield=81.7%).

### Synthesis of C21

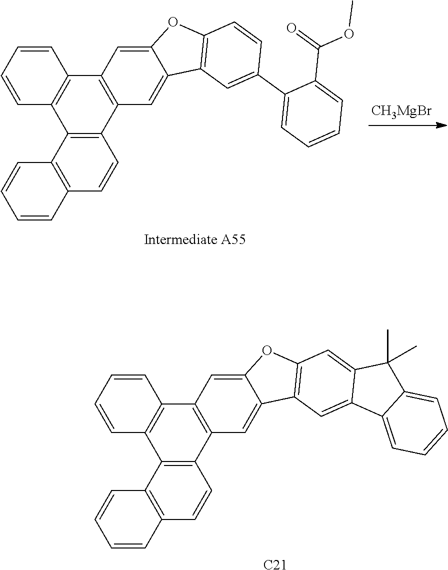

The same synthesis procedure as in Synthesis of Compound C1 was used, except that 2 g of Intermediate A55 was used instead of Intermediate A6 to obtain the desired Compound C21 (0.85 g, yield=44.1%).

### Example 14

### Synthesis of Intermediate A56

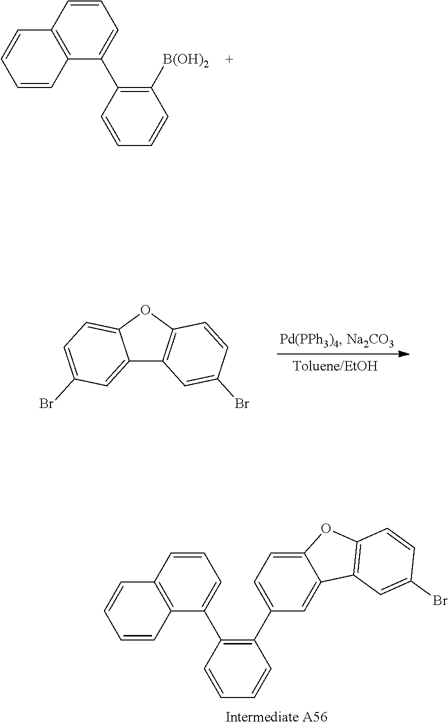 [SMILES: OB(O)c1ccccc1-c1cccc2ccccc12 ; Brc1ccc2oc3ccc(Br)cc3c2c1 ; Brc1ccc2oc3ccc(-c4ccccc4-c4cccc5ccccc45)cc3c2c1]

The same synthesis procedure as in Synthesis of Intermediate A12 was used, except that 2 g of (2-(naphthalen-1-yl)phenyl)boronic acid was used instead of 2-phenylnaphthalen-1-ylboronic acid and 1.4 g of 2,8-dibromodibenzo[b,d]furan was used instead of 1,4-dibromobenzene to obtain the desired Intermediate A56 (1.7 g, yield=43.4%).

### Synthesis of Intermediate A57

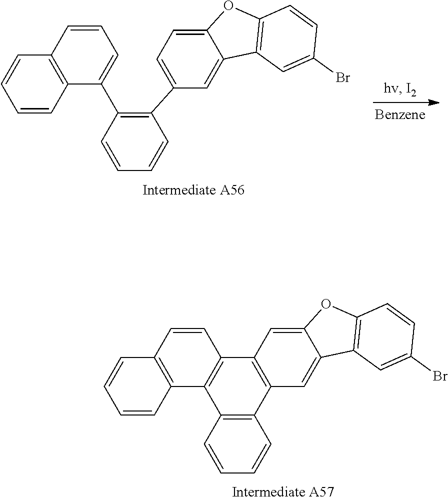 [SMILES: Brc1ccc2oc3ccc(-c4ccccc4-c4cccc5ccccc45)cc3c2c1 ; Brc1ccc2oc3cc4c(cc3c2c1)c1ccccc1c1c2ccccc2ccc41]

A mixture of 10 g (22.3 mmol) of Intermediate A56, 0.06 g (0.23 mmol) of Iodine, and 1000 ml of benzene was degassed and placed under nitrogen, and then exposed to UV light for 4 hrs. After the reaction finished, the solvent was removed, and then the residue was recrystallized 3 times to give Intermediate A57 (2.2 g, 22%).

### Synthesis of Intermediate A58

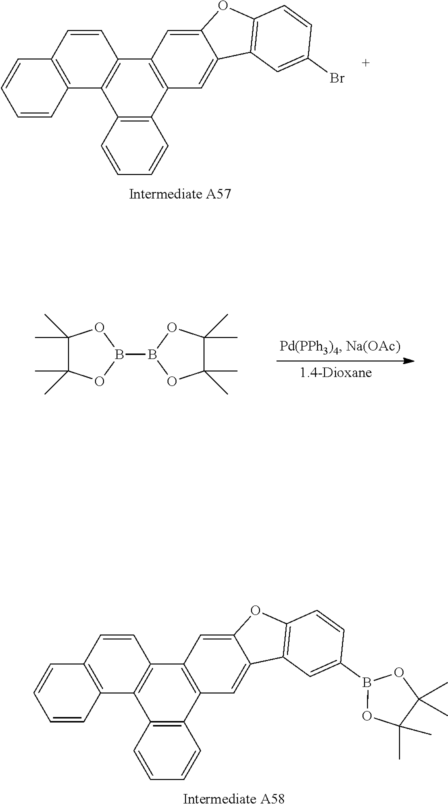

The same synthesis procedure as in Synthesis of Intermediate A17 was used, except that 2 g of Intermediate A57 was used instead of Intermediate A16 to obtain the desired Intermediate A58 (1.5 g, yield=68.2%).

### Synthesis of Intermediate A59

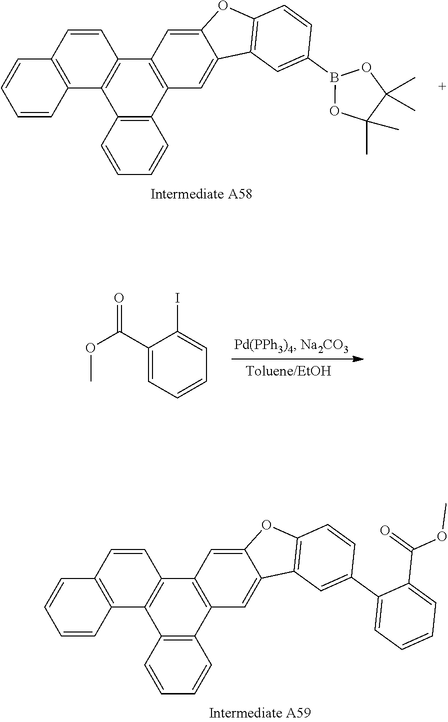 [SMILES: CC1(C)OB(c2ccc3oc4cc5c(cc4c3c2)c2ccccc2c2c3ccccc3ccc52)OC1(C)C ; COC(=O)c1ccccc1I ; COC(=O)c1ccccc1-c1ccc2oc3cc4c(cc3c2c1)c1ccccc1c1c2ccccc2ccc41]

The same synthesis procedure as in Synthesis of Intermediate A6 was used, except that 3 g of Intermediate A58 was used instead of Intermediate A5 to obtain the desired Intermediate A59 (2.3 g, yield=75.2%).

### Synthesis of C24

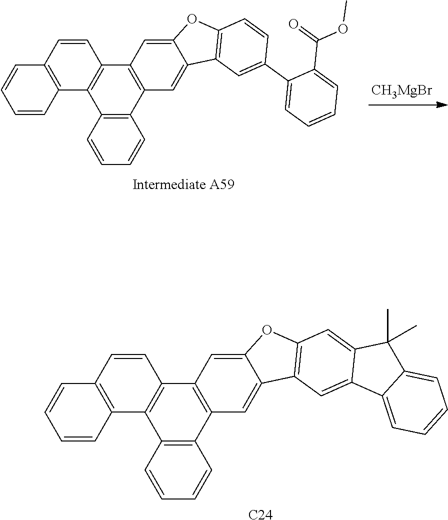

The same synthesis procedure as in Synthesis of Compound C1 was used, except that 2 g of Intermediate A59 was used instead of Intermediate A6 to obtain the desired Compound C24 (0.88 g, yield=45.6%).

### Example 15

### Synthesis of Intermediate A60

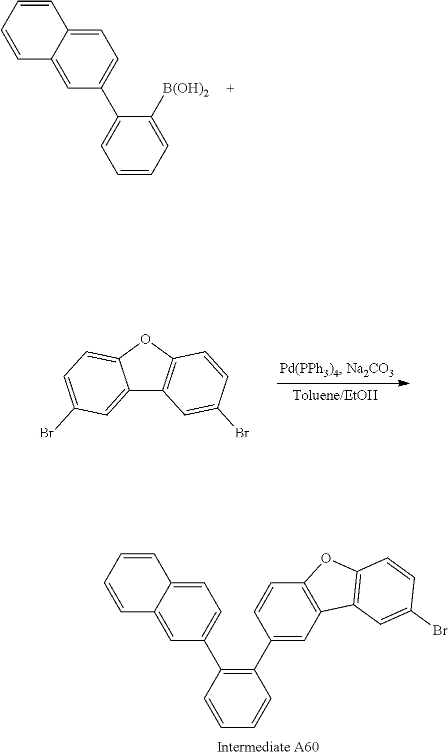 [SMILES: OB(O)c1ccccc1-c1ccc2ccccc2c1 ; Brc1ccc2oc3ccc(Br)cc3c2c1 ; Brc1ccc2oc3ccc(-c4ccccc4-c4ccc5ccccc5c4)cc3c2c1]

The same synthesis procedure as in Synthesis of Intermediate A12 was used, except that 2 g of 2-(naphthalen-2-yl)phenylboronic acid was used instead of 2-phenylnaphthalen-1-ylboronic acid and 2.6 g of 2,8-dibromodibenzo[b,d]furan was used instead of 1,4-dibromobenzene to obtain the desired Intermediate A60 (1.6 g, yield=43.9%).

### Synthesis of Intermediate A61

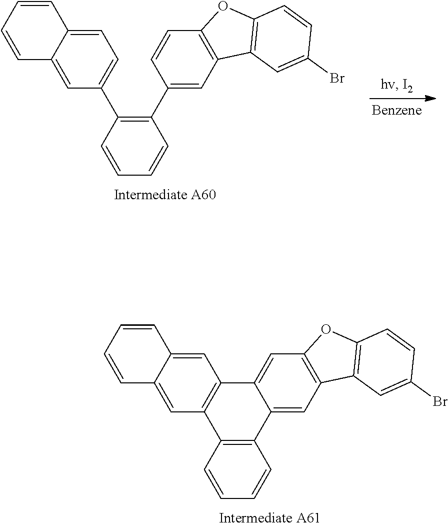 [SMILES: Brc1ccc2oc3ccc(-c4ccccc4-c4ccc5ccccc5c4)cc3c2c1 ; Brc1ccc2oc3cc4c5cc6ccccc6cc5c5ccccc5c4cc3c2c1]

A mixture of 10 g (22.2 mmol) of Intermediate A60, 0.06 g (0.23 mmol) of Iodine, and 1000 ml of benzene was degassed and placed under nitrogen, and then exposed to UV light for 4 hrs. After the reaction finished, the solvent was removed, and then the residue was recrystallized 3 times to give Intermediate A61 (2.3 g, 23%).

### Synthesis of Intermediate A62

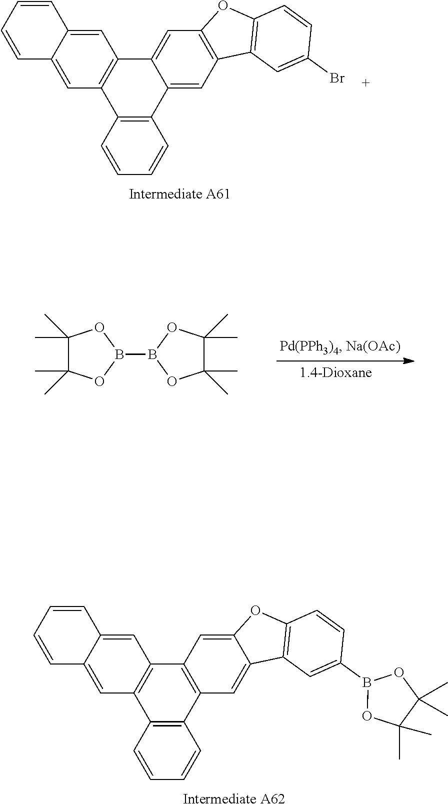

The same synthesis procedure as in Synthesis of Intermediate A17 was used, except that 2 g of Intermediate A61 was used instead of Intermediate A16 to obtain the desired Intermediate A62 (1.3 g, yield=59.1%).

### Synthesis of Intermediate A63

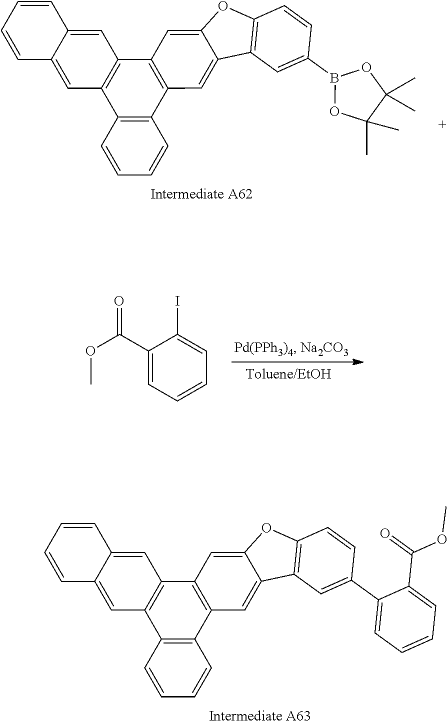 [SMILES: CC1(C)OB(c2ccc3oc4cc5c6cc7ccccc7cc6c6ccccc6c5cc4c3c2)OC1(C)C ; COC(=O)c1ccccc1I ; COC(=O)c1ccccc1-c1ccc2oc3cc4c5cc6ccccc6cc5c5ccccc5c4cc3c2c1]

The same synthesis procedure as in Synthesis of Intermediate A6 was used, except that 3 g of Intermediate A62 was used instead of Intermediate A5 to obtain the desired Intermediate A63 (2.4 g, yield=80%).

### Synthesis of C25

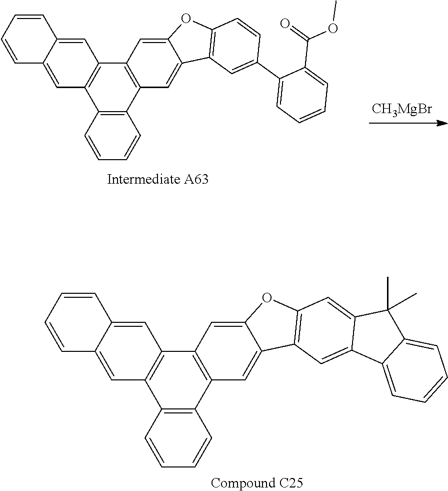

The same synthesis procedure as in Synthesis of Compound C1 was used, except that 2 g of Intermediate A63 was used instead of Intermediate A6 to obtain the desired Compound C25 (0.78 g, yield=40.1%).

### General Method of Producing Organic El Device

ITO-coated glasses with 12 ohm/square in resistance and 120 nm in thickness are provided (hereinafter ITO substrate) and cleaned in a number of cleaning steps in an ultrasonic bath (e.g. detergent, deionized water). Before vapor deposition of the organic layers, cleaned ITO substrates are further treated by UV and ozone. All pre-treatment processes for ITO substrates are under clean room (class 100).

The organic layers are applied onto the ITO substrate in order by vapor deposition in a high-vacuum unit (10⁻⁷ Torr), such as: resistively heated quartz boats. The thickness of the respective layer and the vapor deposition rate (0.1˜0.3 nm/sec) are precisely monitored or set with the aid of a quartz-crystal monitor. It is also possible, as described above, for individual layers to consist of more than one compound, i.e. in general a host material doped with a dopant material. This is successfully achieved by co-vaporization from two or more sources, which means the organic compounds of the present invention are thermally stable.

Dipyrazino[2,3-f:2,3-]quinoxaline-2,3,6,7,10,11-hexacarbonitrile (HAT-CN) is used to form the hole injection layer, and N,N-bis(naphthalene-1-yl)-N,N-bis(phenyl)-benzidine (NPB) is used to form the hole transporting layer of the organic EL device. 2,9-bis(naphthalene-2-yl)-4,7-diphenyl-1,10-phenanthroline (NPhen) is used as the electron transporting material in organic EL device for its high thermal stability and long life-time than BPhen or BCP. For fluorescence emitting device, 1,1′-(9,9-dimethyl-9H-fluorene-2,7-diyl)dipyrene (DFDP) is used as the host material, and (E)-6-(4-(diphenylamino)styryl)-N,N-diphenylnaphthalen-2-amine (D1) is used as the fluorescent dopant. For phosphorescence emitting device, bis(2-methyl-8-quinolinolate)-4-(phenylphenolato)aluminium (BAlq) is used as the host material of emitting layer, and tris(1-phenylisoquinoline)-Iridium(III) (Ir(piq)₃) or tris(2-phenylquinoline)iridium(III) (Ir(2-phq)₃) is used as the dopant material. Compounds C77 and C78 are used as the fluorescent host materials to compare with DFDP. Compounds C71, C72, and C85 are used as the fluorescent dopant materials to compare with D1. Compounds C79, C80, and C89 are used as the electron transporting materials to compare with NPhen. Compounds C1, C2, C4, C6, C21, C24, and C25 are used as the phosphorescent host materials to compare with BAlq. The chemical structures of conventional OLED materials and the exemplary organic compounds of the present invention for producing control and exemplary organic EL devices in this invention are shown as follows:

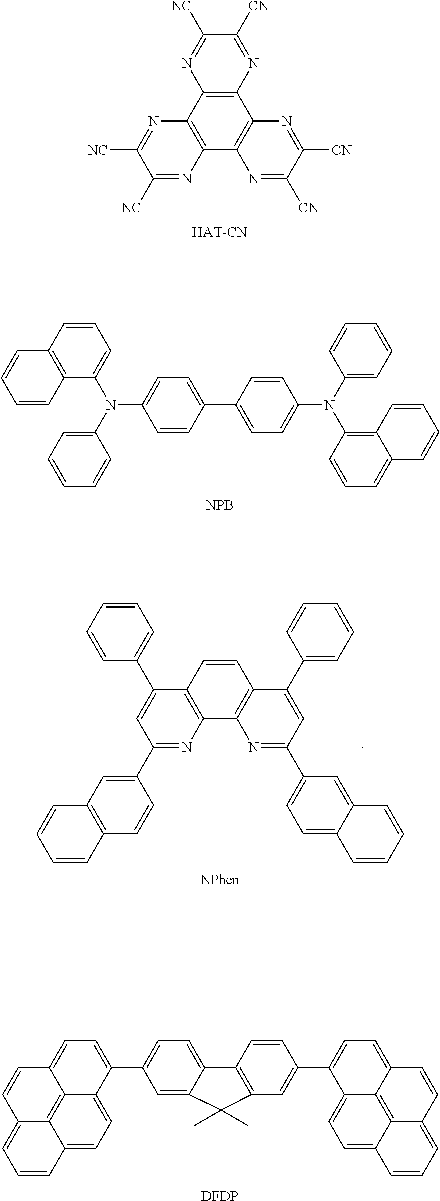

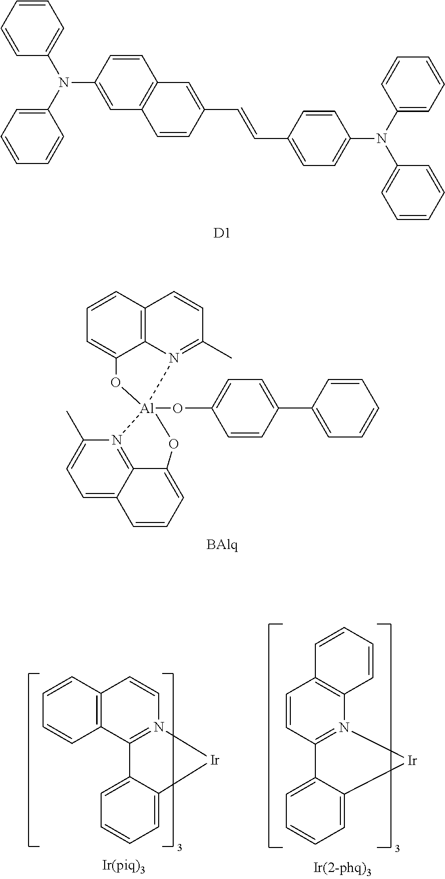

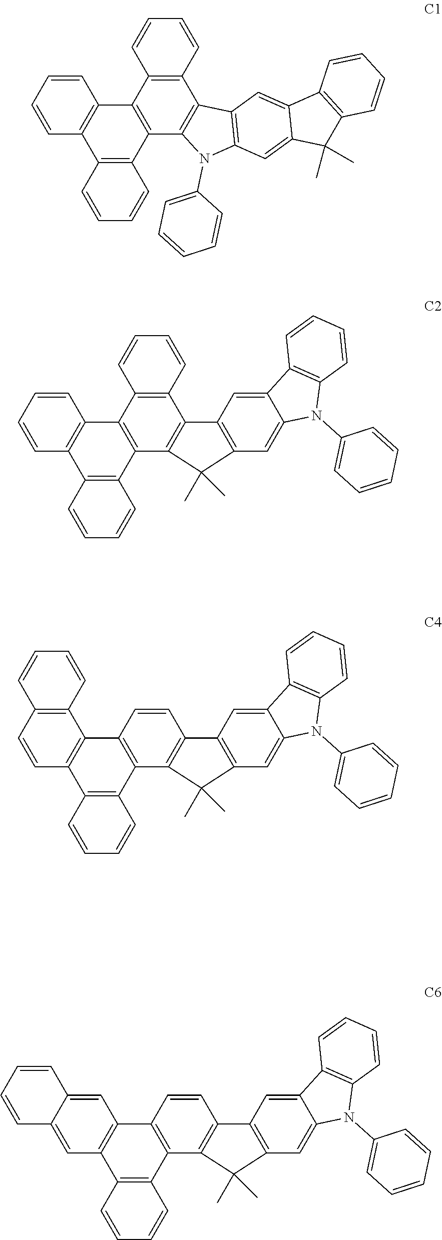 [SMILES: CC1(C)c2ccccc2-c2cc3c4c5ccccc5c5c6ccccc6c6ccccc6c5c4n(-c4ccccc4)c3cc21 ; CC1(C)c2cc3c(cc2-c2c1c1c4ccccc4c4ccccc4c1c1ccccc21)c1ccccc1n3-c1ccccc1 ; CC1(C)c2cc3c(cc2-c2ccc4c(c21)c1ccccc1c1ccc2ccccc2c14)c1ccccc1n3-c1ccccc1 ; CC1(C)c2cc3c(cc2-c2ccc4c5cc6ccccc6cc5c5ccccc5c4c21)c1ccccc1n3-c1ccccc1]

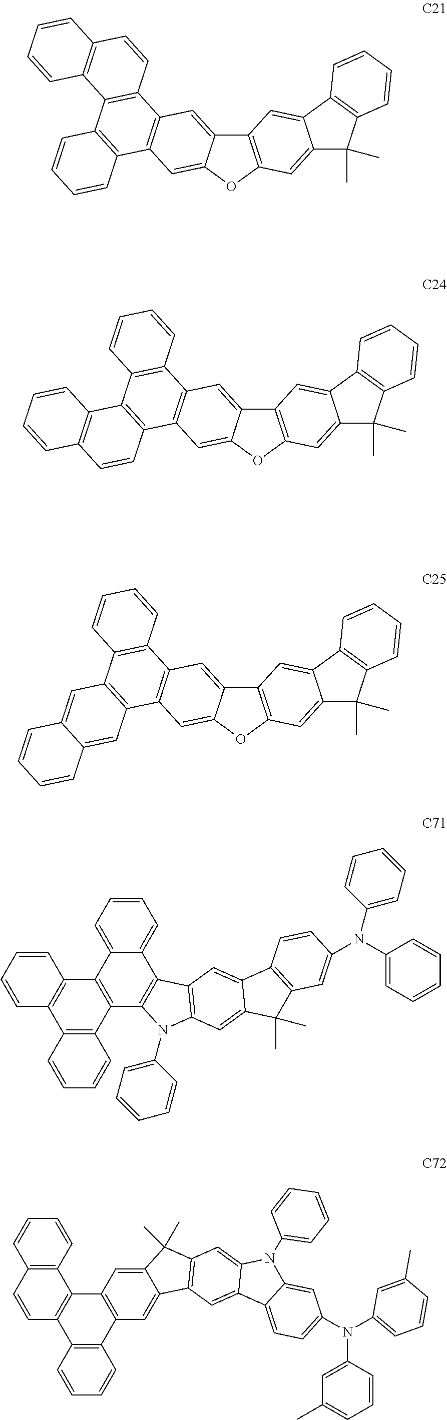 [SMILES: CC1(C)c2ccccc2-c2cc3c(cc21)oc1cc2c4ccccc4c4c5ccccc5ccc4c2cc13 ; CC1(C)c2ccccc2-c2cc3c(cc21)oc1cc2c(cc13)c1ccccc1c1c3ccccc3ccc21 ; CC1(C)c2ccccc2-c2cc3c(cc21)oc1cc2c4cc5ccccc5cc4c4ccccc4c2cc13 ; CC1(C)c2cc(N(c3ccccc3)c3ccccc3)ccc2-c2cc3c4c5ccccc5c5c6ccccc6c6ccccc6c5c4n(-c4ccccc4)c3cc21 ; Cc1cccc(N(c2cccc(C)c2)c2ccc3c4cc5c(cc4n(-c4ccccc4)c3c2)C(C)(C)c2cc3c(cc2-5)c2ccccc2c2ccc4ccccc4c23)c1]

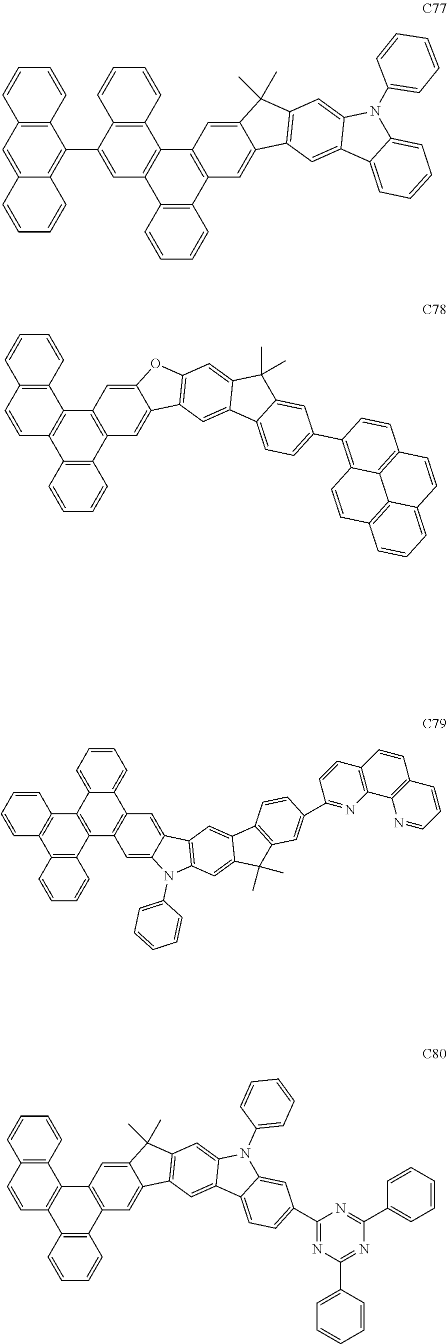 [SMILES: CC1(C)c2cc3c(cc2-c2cc4c5ccccc5n(-c5ccccc5)c4cc21)c1ccccc1c1cc(-c2c4ccccc4cc4ccccc24)c2ccccc2c31 ; CC1(C)c2cc(-c3ccc4ccc5cccc6ccc3c4c56)ccc2-c2cc3c(cc21)oc1cc2c(cc13)c1ccccc1c1ccc3ccccc3c12 ; CC1(C)c2cc(-c3ccc4ccc5cccnc5c4n3)ccc2-c2cc3c4cc5c6ccccc6c6c7ccccc7c7ccccc7c6c5cc4n(-c4ccccc4)c3cc21 ; CC1(C)c2cc3c(cc2-c2cc4c5ccc(-c6nc(-c7ccccc7)nc(-c7ccccc7)n6)cc5n(-c5ccccc5)c4cc21)c1ccccc1c1ccc2ccccc2c13]

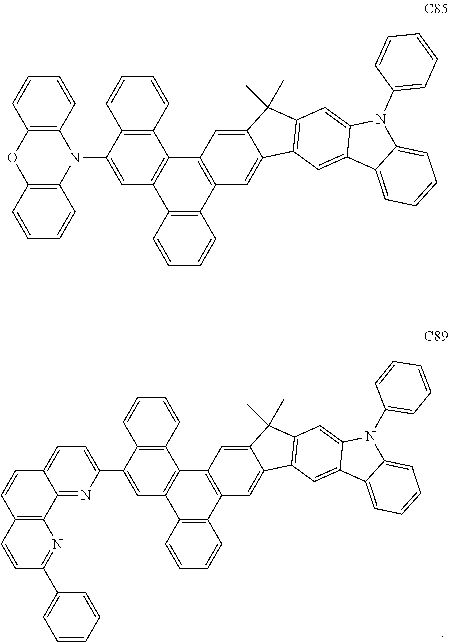 [SMILES: CC1(C)c2cc3c(cc2-c2cc4c5ccccc5n(-c5ccccc5)c4cc21)c1ccccc1c1cc(N2c4ccccc4Oc4ccccc42)c2ccccc2c31 ; CC1(C)c2cc3c(cc2-c2cc4c5ccccc5n(-c5ccccc5)c4cc21)c1ccccc1c1cc(-c2ccc4ccc5ccc(-c6ccccc6)nc5c4n2)c2ccccc2c31]

A typical organic EL device consists of low work function metals, such as A1, Mg, Ca, Li and K, as the cathode by thermal evaporation, and the low work function metals can help electrons injecting the electron transporting layer from cathode. In addition, for reducing the electron injection barrier and improving the organic EL device performance, a thin-film electron injecting layer is introduced between the cathode and the electron transporting layer. Conventional materials of electron injecting layer are metal halide or metal oxide with low work function, such as: LiF, MgO, or Li₂O.

On the other hand, after the organic EL device fabrication, EL spectra and CIE coordination are measured by using a PR650 spectra scan spectrometer. Furthermore, the current/voltage, luminance/voltage, and yield/voltage characteristics are taken with a Keithley 2400 programmable voltage-current source. The above-mentioned apparatuses are operated at room temperature (about 25° C.) and under atmospheric pressure.

### Example 16

Using a procedure analogous to the above-mentioned general method, organic EL devices emitting blue fluorescence and having the following device structure as shown in the FIGURE were produced: ITO/HAT-CN(20 nm)/NPB(50 nm)/fluorescent blue host (DFDP or C77 or C78)+5% dopant(D1 or C71, C72, or C85) (30 nm)/NPhen (30 nm)/LiF (0.5 nm)/A1(160 nm). In the device illustrated in the FIGURE, the hole injection layer 20 is deposited onto the transparent electrode 10, the hole transport layer 30 is deposited onto the hole injection layer 20, the emitting layer 40 is deposited onto the hole transport layer 30, the electron transport layer 50 is deposited onto the emitting layer 40, the electron injection layer 60 is deposited onto the electron transport layer 50, and the metal electrode 70 is deposited onto the electron injection layer 60. The I—V—B and half-life time test reports of these fluorescent blue-emitting organic EL devices are summarized in Table 1 below, and the half-life time is defined as the time the initial luminance of 3000 cd/m² has dropped to half.

TABLE 1

| Fluorescent | | | | | |
|---|---|---|---|---|---|
| blue host + | Voltage | Luminance | Efficiency | CIE | Half-life |
| 5% dopant | (V) | (cd/m²) | (cd/A) | (y) | time (hr) |
| DFDP + D1 | 6 | 965 | 5.02 | 0.17 | 300 |
| DFDP + C71 | 6 | 1506 | 7.62 | 0.14 | 480 |
| DFDP + C72 | 6 | 1100 | 5.50 | 0.15 | 450 |
| DFDP + C85 | 6 | 1036 | 6.10 | 0.15 | 421 |
| C77 + D1 | 6 | 1126 | 6.18 | 0.15 | 388 |
| C78 + D1 | 6 | 1340 | 6.53 | 0.15 | 410 |

From the above test report summary of the organic EL devices, it is obvious that the organic compound of formula (1) or formula (2) used as the fluorescent blue host or dopant material exhibits better performance than the prior art materials. In particular, the organic EL devices of the present invention employing the organic compound of formula (1) or formula (2) as the dopant material or host material to collocate with the host material DFDP or the dopant material D1 have improved luminance, current efficiency, and half-life time under the same voltage.

### Example 17

Using a procedure analogous to the above-mentioned general method, organic EL devices having the following device structure were produced: ITO/HAT-CN(20 nm)/NPB(50 nm)/DFDP+5% D1 (30 nm)/NPhen or C79, C80, or C89(30 nm)/LiF(0.5 nm)/A1(160 nm). The I—V—B and half-life time test reports of these blue fluorescence-emitting organic EL devices are summarized in Table 2 below, and the half-life time is defined as the time the initial luminance of 3000 cd/m² has dropped to half.

TABLE 2

| | | Efficiency | CIE | Half-life |
|---|---|---|---|---|
| ETM | Voltage (V) | (cd/A) | (y) | time (hr) |
| NPhen | 6.2 | 5.12 | 0.17 | 310 |
| C79 | 4.3 | 8.60 | 0.17 | 425 |
| C80 | 4.6 | 7.82 | 0.17 | 463 |
| C89 | 5.0 | 8.43 | 0.17 | 560 |

From the summary of the test report the above organic EL devices, it can be seen that the organic compound of formula (1) or formula (2) used as the electron transporting material exhibits better performance than the prior art material NPhen. In particular, the organic EL device of the present invention using the organic compound of formula (1) or formula (2) as the electron transporting material to collocate with the host material DFDP and the dopant material D1 has lower power consumption, higher current efficiency, and longer half-life time.

### Example 18

Using a procedure analogous to the above-mentioned general method, organic EL devices emitting phosphorescence and having the following device structure were produced: ITO/HAT-CN(20 nm)/NPB(50 nm)/phosphorescent host (C1, C2, C4)+10% dopant (30 nm)/NPhen (30 nm)/LiF(0.5 nm)/A1(160 nm). The I—V—B and half-life time test reports of these phosphorescence emitting organic EL devices are summarized in Table 3 below, and the half-life time is defined as the time the initial luminance of 3000 cd/m² has dropped to half.

TABLE 3

| Phosphorescent | Vol- | | | | |
|---|---|---|---|---|---|
| host + 10% | tage | Luminance | Efficiency | Device | Half-life |
| dopant | (V) | (cd/m²) | (cd/A) | color | time (hr) |
| BAlq + Ir(piq)₃ | 6 | 671 | 8.14 | red | 450 |
| C1 + Ir(piq)₃ | 6 | 1030 | 12.55 | red | 785 |
| C2 + Ir(piq)₃ | 6 | 950 | 11.19 | red | 680 |
| C4 + Ir(piq)₃ | 6 | 1241 | 15.05 | red | 804 |
| C6 + Ir(piq)₃ | 6 | 1010 | 13.10 | red | 810 |
| C21 + Ir(piq)₃ | 6 | 880 | 11.54 | red | 710 |
| C24 + Ir(piq)₃ | 6 | 910 | 11.31 | red | 750 |
| C25 + Ir(piq)₃ | 6 | 1180 | 14.32 | red | 803 |
| BAlq + Ir(2-phq)₃ | 6 | 511 | 14.12 | yellow | 472 |
| C1 + Ir(2-phq)₃ | 6 | 1180 | 35.36 | yellow | 1006 |
| C2 + Ir(2-phq)₃ | 6 | 1220 | 31.26 | yellow | 990 |
| C4 + Ir(2-phq)₃ | 6 | 1311 | 36.22 | yellow | 1136 |
| C6 + Ir(2-phq)₃ | 6 | 1263 | 34.31 | yellow | 1011 |
| C21 + Ir(2-phq)₃ | 6 | 1050 | 30.31 | yellow | 888 |
| C24 + Ir(2-phq)₃ | 6 | 1082 | 32.63 | yellow | 850 |
| C25 + Ir(2-phq)₃ | 6 | 1169 | 35.31 | yellow | 998 |

From the above test report summary of the organic EL devices, it is evident that the organic compound of formula (1) or formula (2) used as the phosphorescent host material has better performance than the prior art material BAlq. The organic EL devices of the present invention using the organic compound of formula (1) or formula (2) as the phosphorescent host material to collocate with the dopant material Ir(piq)₃ or Ir(2-phq)₃ have superior luminance and current efficiency and extended half-life time under the same voltage.

To sum up, the present invention discloses an organic compound, which can be used as the phosphorescent host material, the fluorescent host material, or the fluorescent dopant material of the light emitting layer, or the electron transporting material in organic EL devices. The mentioned organic compound is represented by the following formula (1) or formula (2):

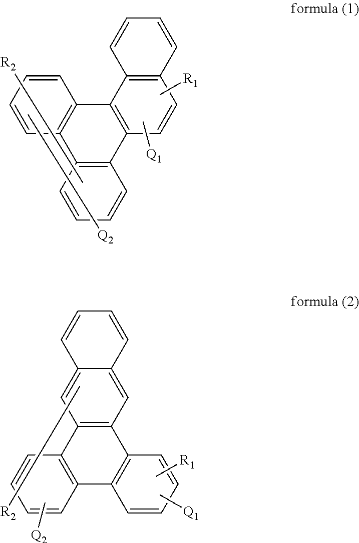

wherein one of Q₁ and Q₂ represents formula (3) below:

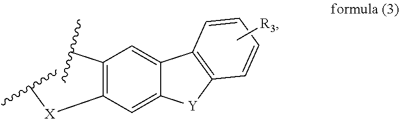

wherein X and Y are independently a divalent bridge selected from the group consisting of O, S, Se, NR₄, CR₅R₆, and SiR₇R₈; R₁ to R₃ are independently absent, a hydrogen atom, a halide, a substituted or unsubstituted aryl group having 5 to 50 ring atoms, a substituted or unsubstituted heteroaryl group having 5 to 50 ring atoms, a substituted or unsubstituted arylamine group having 5 to 50 ring atoms, or a substituted or unsubstituted heteroarylamine group having 5 to 50 ring atoms; R₄ is a hydrogen atom, a halide, a substituted or unsubstituted aryl group having 5 to 50 ring atoms, a substituted or unsubstituted heteroaryl group having 5 to 50 ring atoms, a substituted or unsubstituted arylamine group having 5 to 50 ring atoms, or a substituted or unsubstituted heteroarylamine group having 5 to 50 ring atoms; and R₅ to R₈ are independently a hydrogen atom, a halide, a substituted or unsubstituted alkyl group having 1 to 30 carbon atoms, a substituted or unsubstituted aryl group having 6 to 30 carbon atoms, a substituted or unsubstituted aralkyl group having 6 to 30 carbon atoms, or a substituted or unsubstituted heteroaryl group having 3 to 30 carbon atoms.

Obviously, many modifications and variations are possible in light of the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

What is claimed is:

1. An organic compound of formula (1) or formula (2) below: 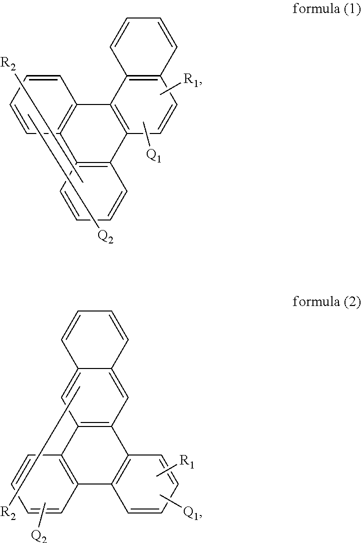
wherein one of Q₁ and Q₂ represents formula (3) below: 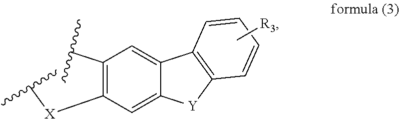
wherein X and Y are independently a divalent bridge selected from the group consisting of O, S, Se, NR₄, CR₅R₆, and SiR₇R₉; R₁ to R₃ are independently absent, a hydrogen atom, a halide, a substituted or unsubstituted aryl group having 5 to 50 ring atoms, a substituted or unsubstituted heteroaryl group having 5 to 50 ring atoms, a substituted or unsubstituted arylamine group having 5 to 50 ring atoms, or a substituted or unsubstituted heteroarylamine group having 5 to 50 ring atoms; R₄ is a hydrogen atom, a halide, a substituted or unsubstituted aryl group having 5 to 50 ring atoms, a substituted or unsubstituted heteroaryl group having 5 to 50 ring atoms, a substituted or unsubstituted arylamine group having 5 to 50 ring atoms, or a substituted or unsubstituted heteroarylamine group having 5 to 50 ring atoms; and R₅ to R₈ are independently a hydrogen atom, a halide, a substituted or unsubstituted alkyl group having 1 to 30 carbon atoms, a substituted or unsubstituted aryl group having 6 to 30 carbon atoms, a substituted or unsubstituted aralkyl group having 6 to 30 carbon atoms, or a substituted or unsubstituted heteroaryl group having 3 to 30 carbon atoms.

2. The organic compound according to claim 1, wherein the organic compound is represented by one of the following formula (4) to formula (11): 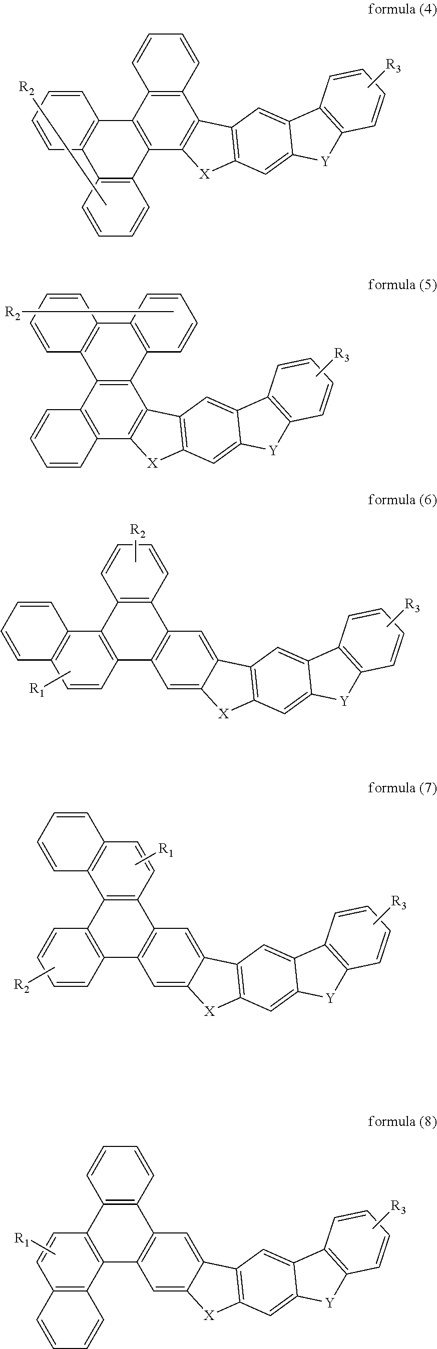 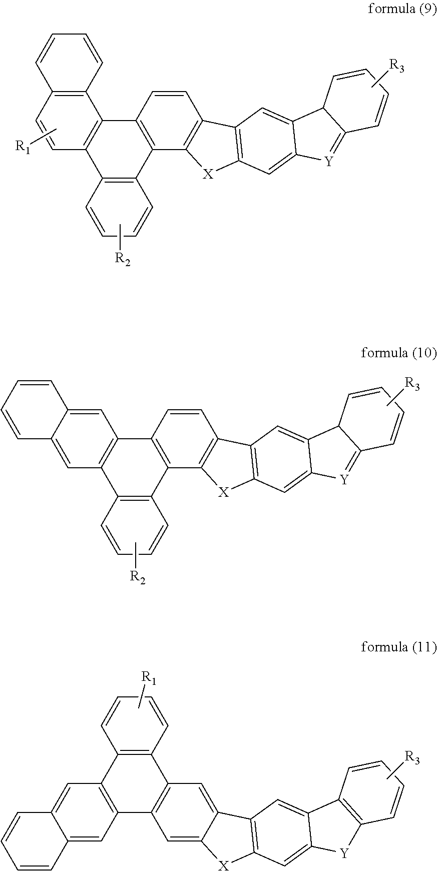
wherein X and Y are independently a divalent bridge selected from the group consisting of O, S, Se, NR₄, CR₅R₆, and SiR₇R₈; R₁ to R₃ are independently absent, a hydrogen atom, a halide, a substituted or unsubstituted aryl group having 5 to 50 ring atoms, a substituted or unsubstituted heteroaryl group having 5 to 50 ring atoms, a substituted or unsubstituted arylamine group having 5 to 50 ring atoms, or a substituted or unsubstituted heteroarylamine group having 5 to 50 ring atoms; R₄ is a hydrogen atom, a halide, a substituted or unsubstituted aryl group having 5 to 50 ring atoms, a substituted or unsubstituted heteroaryl group having 5 to 50 ring atoms, a substituted or unsubstituted arylamine group having 5 to 50 ring atoms, or a substituted or unsubstituted heteroarylamine group having 5 to 50 ring atoms; and R₅ to R₈ are independently a hydrogen atom, a halide, a substituted or unsubstituted alkyl group having 1 to 30 carbon atoms, a substituted or unsubstituted aryl group having 6 to 30 carbon atoms, a substituted or unsubstituted aralkyl group having 6 to 30 carbon atoms, or a substituted or unsubstituted heteroaryl group having 3 to 30 carbon atoms.

3. The organic compound according to claim 1, wherein R₁ to R₄ are independently a substituted or unsubstituted phenyl group, a substituted or unsubstituted biphenyl group, a substituted or unsubstituted fluorene group, a substituted or unsubstituted benzofluorene group, a substituted or unsubstituted naphthyl group, a substituted or unsubstituted anthracenyl group, a substituted or unsubstituted phenanthrenyl group, a substituted or unsubstituted pyrenyl group, a substituted or unsubstituted chrysenyl group, a substituted or unsubstituted triphenylenyl group, a substituted or unsubstituted benzimidazole group, a substituted or unsubstituted carbazolyl group, a substituted or unsubstituted biscarbazolyl group, a substituted or unsubstituted dibenzofuranyl group, a substituted or unsubstituted dibenzothiophenyl group, a substituted or unsubstituted diphenylphosphine oxide group, a substituted or unsubstituted triazinyl group, a substituted or unsubstituted diazinyl group, a substituted or unsubstituted pyridinyl group, a substituted or unsubstituted phenanthroline group, a substituted or unsubstituted dihydroacridine group, a substituted or unsubstituted phenothiazine group, a substituted or unsubstituted phenoxazine group, a substituted or unsubstituted dihydrophenazine group, a substituted or unsubstituted diphenylamine group, a substituted or unsubstituted triphenylamine group, a substituted or unsubstituted phenyldibenzofuranylamine group, or a substituted or unsubstituted phenyldibenzothiophenylamine group.

4. The organic compound according to claim 1, wherein R₁ to R₄ independently represent one of the following substituents:

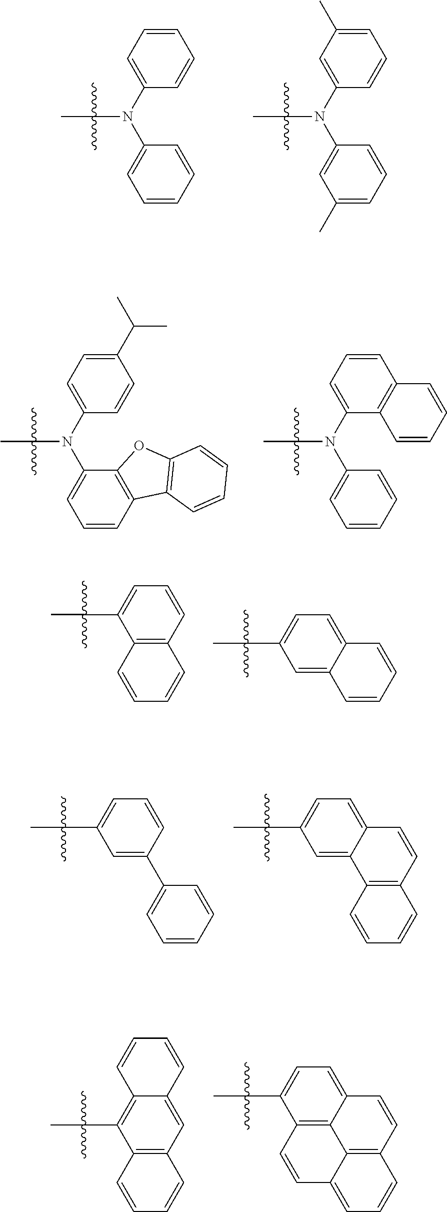

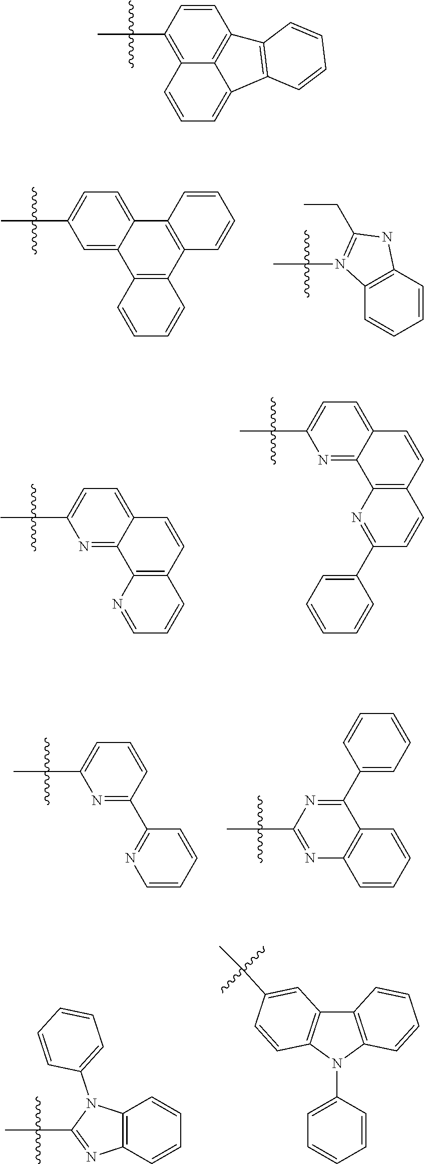

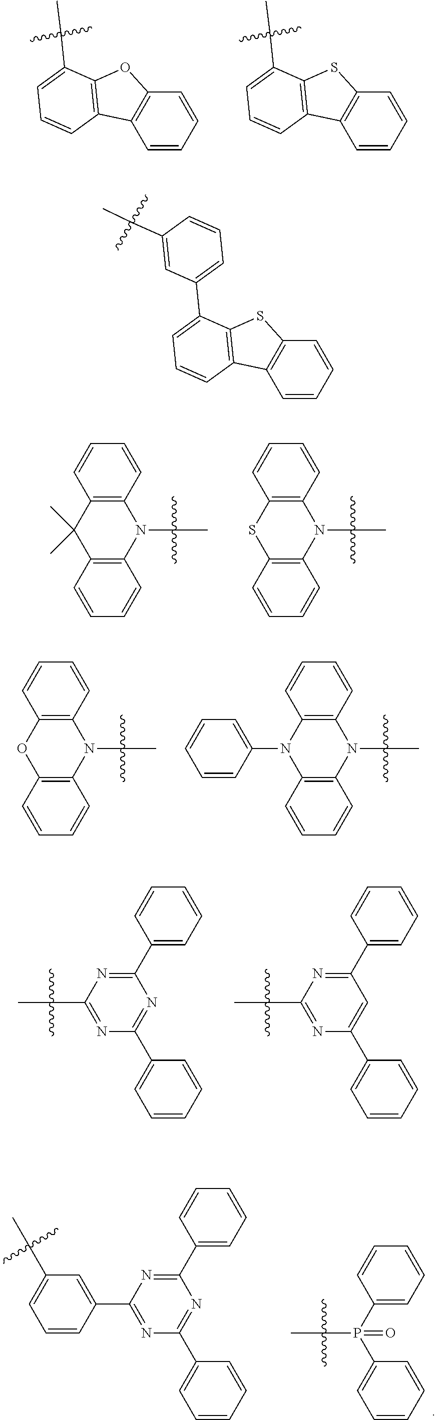

5. The organic compound according to claim 1, wherein the organic compound is one of the following compounds:

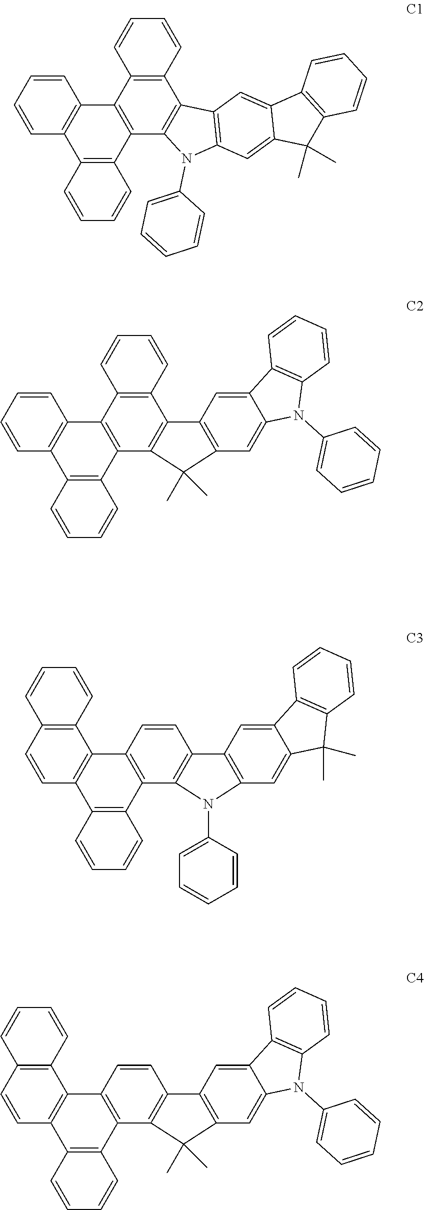 [SMILES: CC1(C)c2ccccc2-c2cc3c4c5ccccc5c5c6ccccc6c6ccccc6c5c4n(-c4ccccc4)c3cc21 ; CC1(C)c2cc3c(cc2-c2c1c1c4ccccc4c4ccccc4c1c1ccccc21)c1ccccc1n3-c1ccccc1 ; CC1(C)c2ccccc2-c2cc3c4ccc5c6c7ccccc7ccc6c6ccccc6c5c4n(-c4ccccc4)c3cc21 ; CC1(C)c2cc3c(cc2-c2ccc4c(c21)c1ccccc1c1ccc2ccccc2c14)c1ccccc1n3-c1ccccc1]

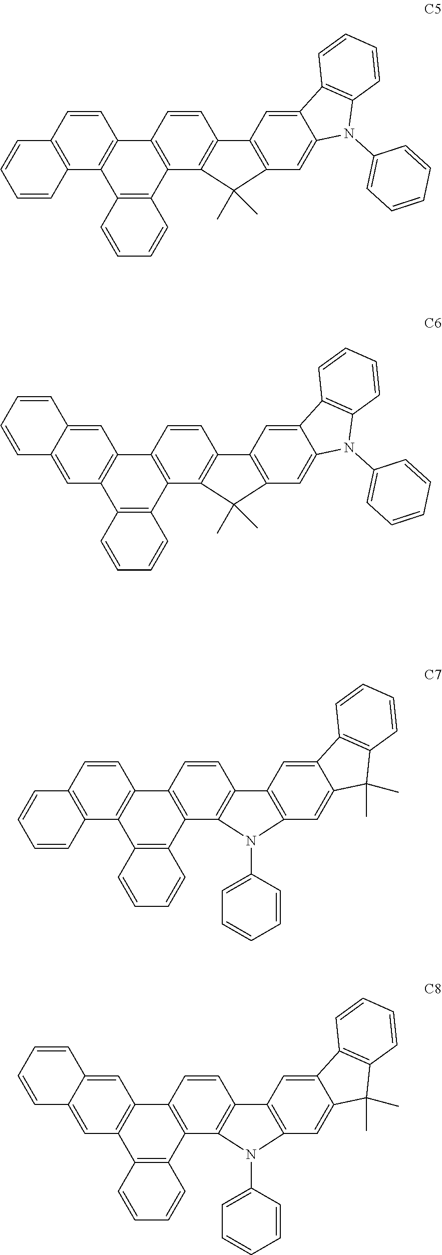 [SMILES: CC1(C)c2cc3c(cc2-c2ccc4c5ccc6ccccc6c5c5ccccc5c4c21)c1ccccc1n3-c1ccccc1 ; CC1(C)c2cc3c(cc2-c2ccc4c5cc6ccccc6cc5c5ccccc5c4c21)c1ccccc1n3-c1ccccc1 ; CC1(C)c2ccccc2-c2cc3c4ccc5c6ccc7ccccc7c6c6ccccc6c5c4n(-c4ccccc4)c3cc21 ; CC1(C)c2ccccc2-c2cc3c4ccc5c6cc7ccccc7cc6c6ccccc6c5c4n(-c4ccccc4)c3cc21]

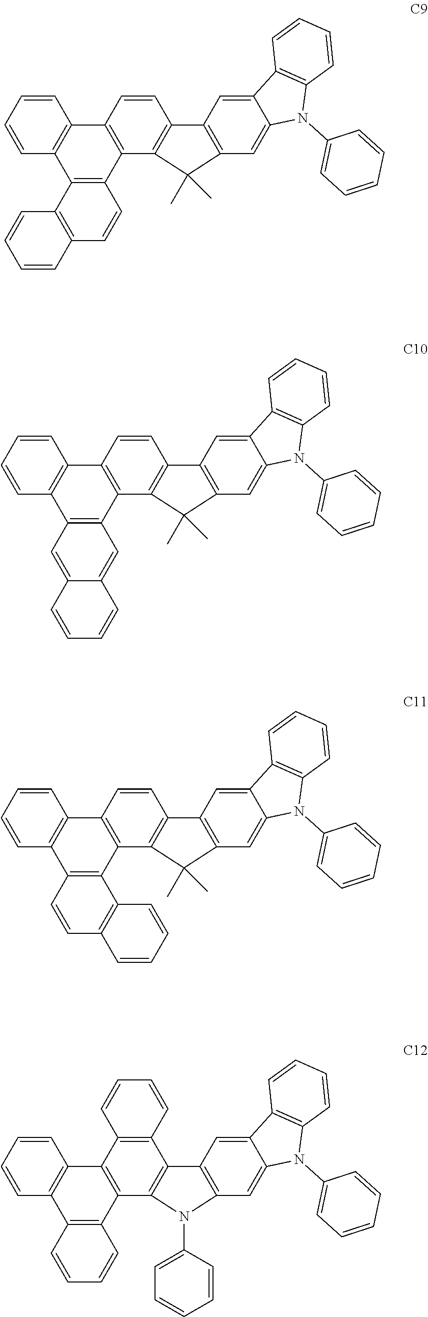 [SMILES: CC1(C)c2cc3c(cc2-c2ccc4c5ccccc5c5c6ccccc6ccc5c4c21)c1ccccc1n3-c1ccccc1 ; CC1(C)c2cc3c(cc2-c2ccc4c5ccccc5c5cc6ccccc6cc5c4c21)c1ccccc1n3-c1ccccc1 ; CC1(C)c2cc3c(cc2-c2ccc4c5ccccc5c5ccc6ccccc6c5c4c21)c1ccccc1n3-c1ccccc1 ; c1ccc(-n2c3ccccc3c3cc4c5c6ccccc6c6c7ccccc7c7ccccc7c6c5n(-c5ccccc5)c4cc32)cc1]

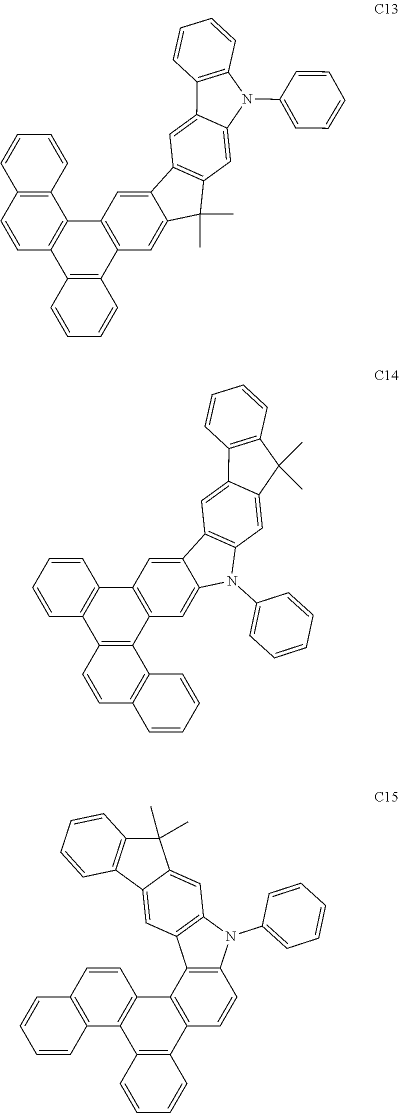 [SMILES: CC1(C)c2cc3c4ccccc4c4ccc5ccccc5c4c3cc2-c2cc3c4ccccc4n(-c4ccccc4)c3cc21 ; CC1(C)c2ccccc2-c2cc3c4cc5c6ccccc6c6ccc7ccccc7c6c5cc4n(-c4ccccc4)c3cc21 ; CC1(C)c2ccccc2-c2cc3c4c5c(ccc4n(-c4ccccc4)c3cc21)c1ccccc1c1c2ccccc2ccc15]

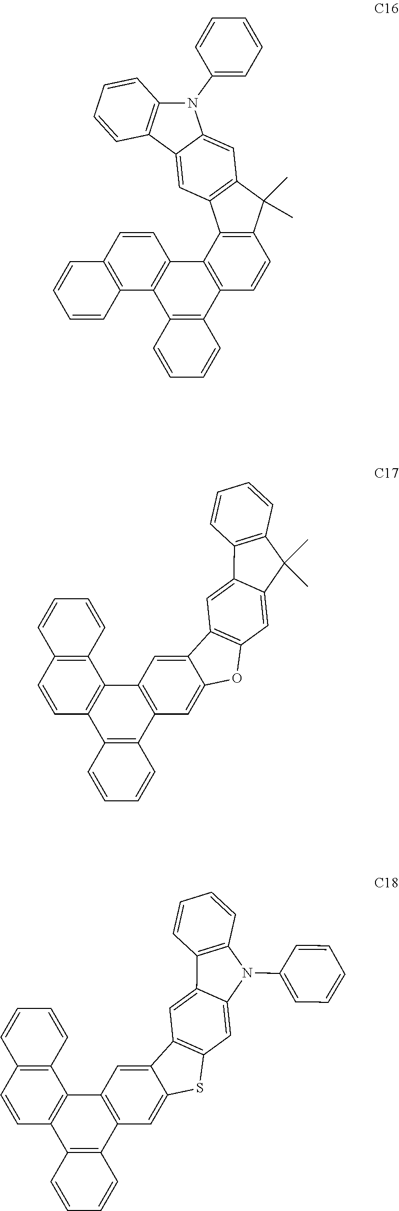 [SMILES: CC1(C)c2cc3c(cc2-c2c1ccc1c4ccccc4c4c5ccccc5ccc4c21)c1ccccc1n3-c1ccccc1 ; CC1(C)c2ccccc2-c2cc3c(cc21)oc1cc2c4ccccc4c4ccc5ccccc5c4c2cc13 ; c1ccc(-n2c3ccccc3c3cc4c(cc32)sc2cc3c5ccccc5c5ccc6ccccc6c5c3cc24)cc1]

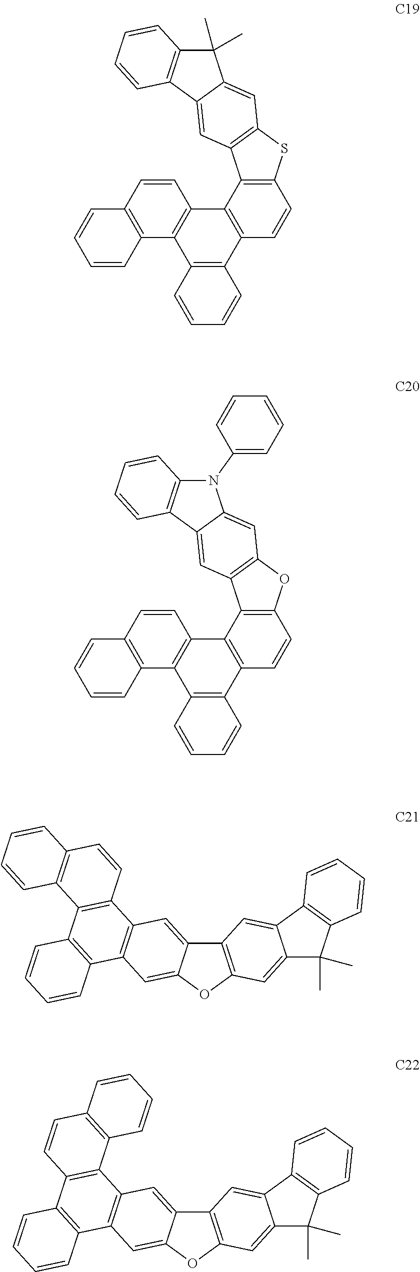 [SMILES: CC1(C)c2ccccc2-c2cc3c(cc21)sc1ccc2c4ccccc4c4c5ccccc5ccc4c2c13 ; c1ccc(-n2c3ccccc3c3cc4c(cc32)oc2ccc3c5ccccc5c5c6ccccc6ccc5c3c24)cc1 ; CC1(C)c2ccccc2-c2cc3c(cc21)oc1cc2c4ccccc4c4c5ccccc5ccc4c2cc13 ; CC1(C)c2ccccc2-c2cc3c(cc21)oc1cc2c4ccccc4c4ccc5ccccc5c4c2cc13]

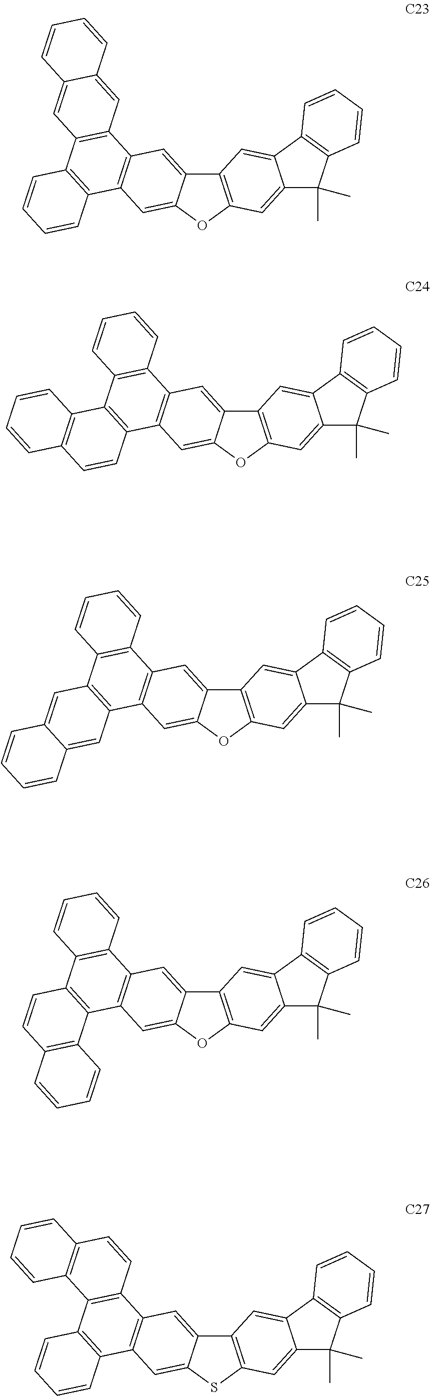 [SMILES: CC1(C)c2ccccc2-c2cc3c(cc21)oc1cc2c4ccccc4c4cc5ccccc5cc4c2cc13 ; CC1(C)c2ccccc2-c2cc3c(cc21)oc1cc2c(cc13)c1ccccc1c1c3ccccc3ccc21 ; CC1(C)c2ccccc2-c2cc3c(cc21)oc1cc2c4cc5ccccc5cc4c4ccccc4c2cc13 ; CC1(C)c2ccccc2-c2cc3c(cc21)oc1cc2c(cc13)c1ccccc1c1ccc3ccccc3c12 ; CC1(C)c2ccccc2-c2cc3c(cc21)sc1cc2c4ccccc4c4c5ccccc5ccc4c2cc13]

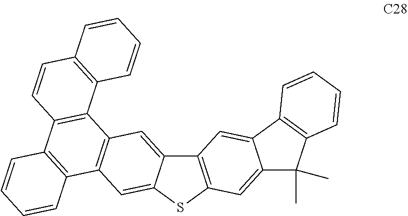 [SMILES: CC1(C)c2ccccc2-c2cc3c(cc21)sc1cc2c4ccccc4c4ccc5ccccc5c4c2cc13]

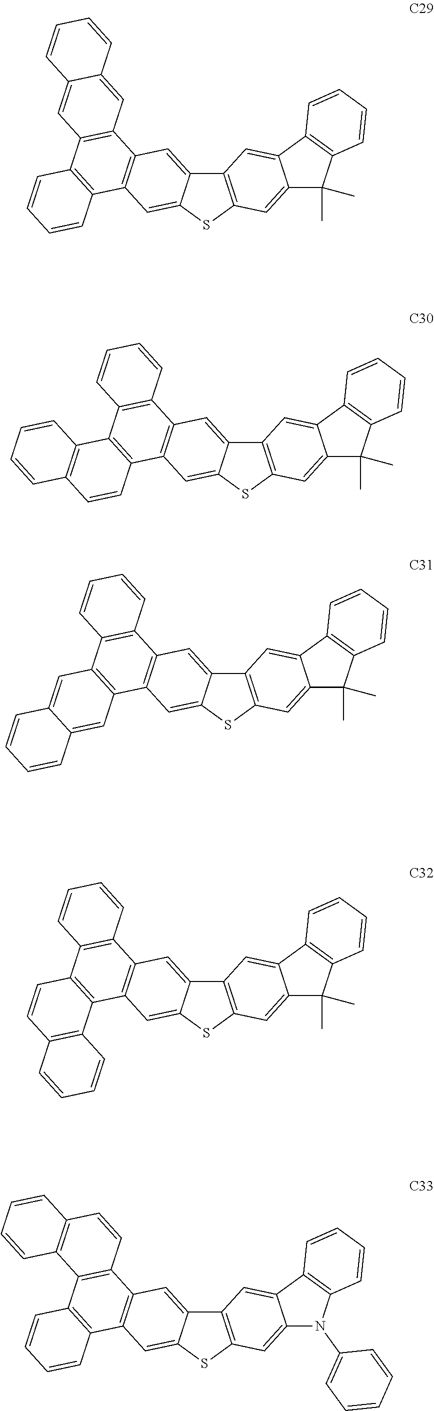 [SMILES: CC1(C)c2ccccc2-c2cc3c(cc21)sc1cc2c4ccccc4c4cc5ccccc5cc4c2cc13 ; CC1(C)c2ccccc2-c2cc3c(cc21)sc1cc2c(cc13)c1ccccc1c1c3ccccc3ccc21 ; CC1(C)c2ccccc2-c2cc3c(cc21)sc1cc2c4cc5ccccc5cc4c4ccccc4c2cc13 ; CC1(C)c2ccccc2-c2cc3c(cc21)sc1cc2c(cc13)c1ccccc1c1ccc3ccccc3c12 ; c1ccc(-n2c3ccccc3c3cc4c(cc32)sc2cc3c5ccccc5c5c6ccccc6ccc5c3cc24)cc1]

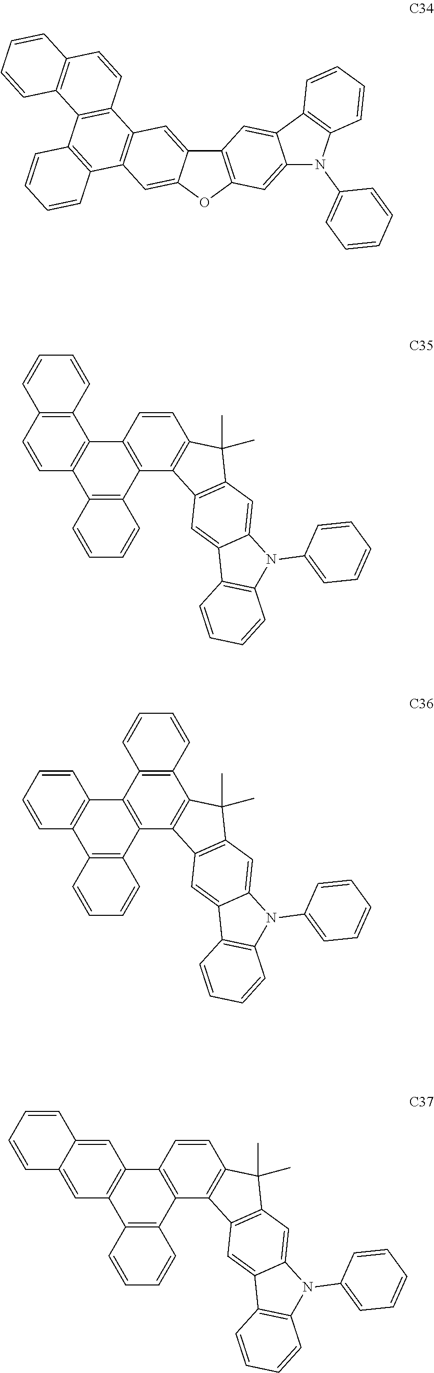 [SMILES: c1ccc(-n2c3ccccc3c3cc4c(cc32)oc2cc3c5ccccc5c5c6ccccc6ccc5c3cc24)cc1 ; CC1(C)c2cc3c(cc2-c2c1ccc1c2c2ccccc2c2ccc4ccccc4c21)c1ccccc1n3-c1ccccc1 ; CC1(C)c2cc3c(cc2-c2c1c1ccccc1c1c4ccccc4c4ccccc4c21)c1ccccc1n3-c1ccccc1 ; CC1(C)c2cc3c(cc2-c2c1ccc1c4cc5ccccc5cc4c4ccccc4c21)c1ccccc1n3-c1ccccc1]

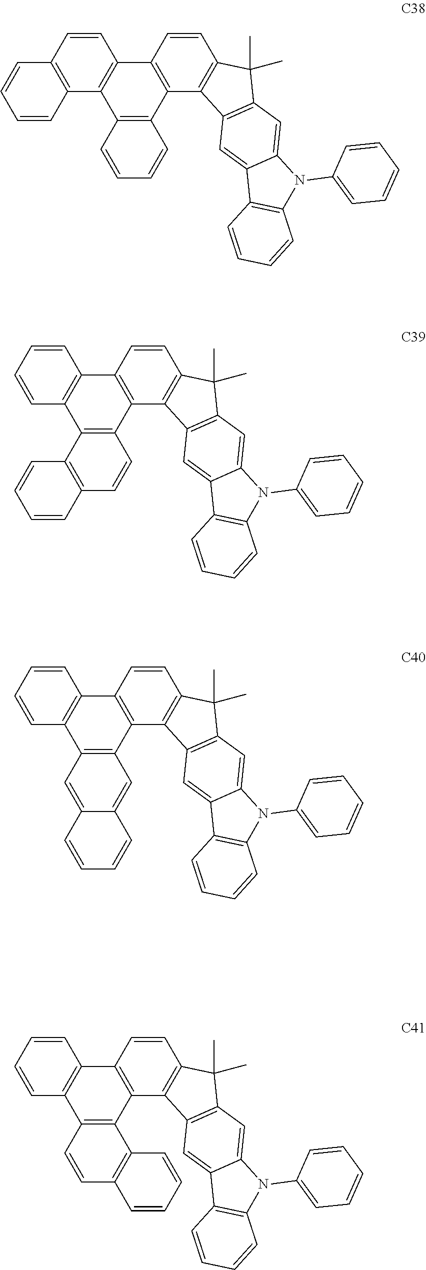 [SMILES: CC1(C)c2cc3c(cc2-c2c1ccc1c4ccc5ccccc5c4c4ccccc4c21)c1ccccc1n3-c1ccccc1 ; CC1(C)c2cc3c(cc2-c2c1ccc1c4ccccc4c4c5ccccc5ccc4c21)c1ccccc1n3-c1ccccc1 ; CC1(C)c2cc3c(cc2-c2c1ccc1c4ccccc4c4cc5ccccc5cc4c21)c1ccccc1n3-c1ccccc1 ; CC1(C)c2cc3c(cc2-c2c1ccc1c4ccccc4c4ccc5ccccc5c4c21)c1ccccc1n3-c1ccccc1]

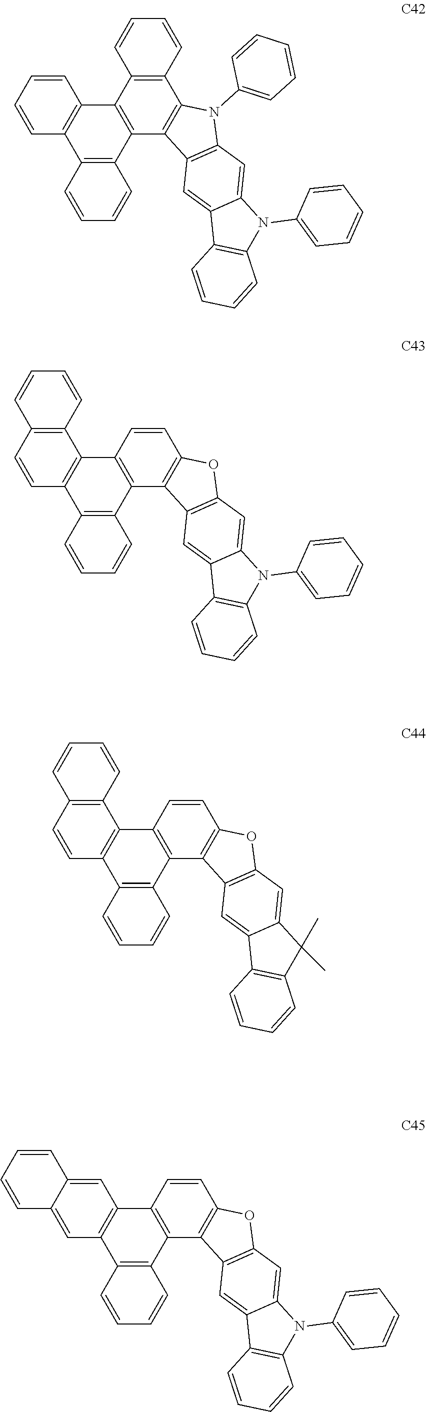 [SMILES: c1ccc(-n2c3ccccc3c3cc4c5c6c7ccccc7c7ccccc7c6c6ccccc6c5n(-c5ccccc5)c4cc32)cc1 ; c1ccc(-n2c3ccccc3c3cc4c(cc32)oc2ccc3c5c6ccccc6ccc5c5ccccc5c3c24)cc1 ; CC1(C)c2ccccc2-c2cc3c(cc21)oc1ccc2c4c5ccccc5ccc4c4ccccc4c2c13 ; c1ccc(-n2c3ccccc3c3cc4c(cc32)oc2ccc3c5cc6ccccc6cc5c5ccccc5c3c24)cc1]

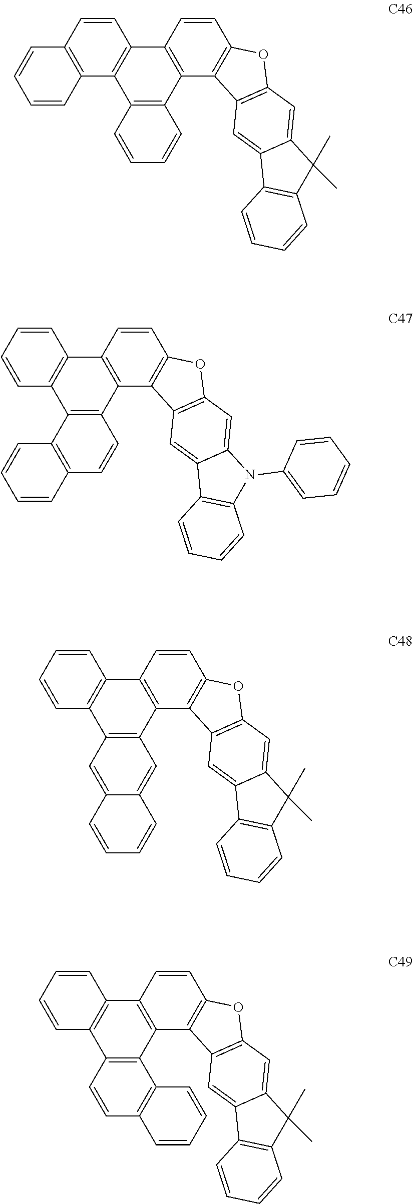 [SMILES: CC1(C)c2ccccc2-c2cc3c(cc21)oc1ccc2c4ccc5ccccc5c4c4ccccc4c2c13 ; c1ccc(-n2c3ccccc3c3cc4c(cc32)oc2ccc3c5ccccc5c5c6ccccc6ccc5c3c24)cc1 ; CC1(C)c2ccccc2-c2cc3c(cc21)oc1ccc2c4ccccc4c4cc5ccccc5cc4c2c13 ; CC1(C)c2ccccc2-c2cc3c(cc21)oc1ccc2c4ccccc4c4ccc5ccccc5c4c2c13]

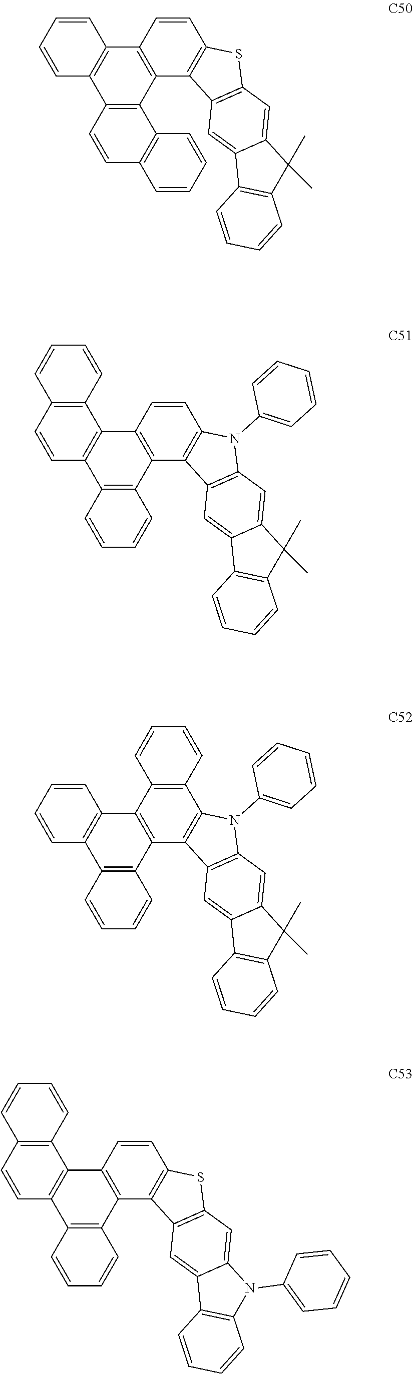 [SMILES: CC1(C)c2ccccc2-c2cc3c(cc21)sc1ccc2c4ccccc4c4ccc5ccccc5c4c2c13 ; CC1(C)c2ccccc2-c2cc3c4c5c6ccccc6c6ccc7ccccc7c6c5ccc4n(-c4ccccc4)c3cc21 ; CC1(C)c2ccccc2-c2cc3c4c5c6ccccc6c6ccccc6c5c5ccccc5c4n(-c4ccccc4)c3cc21 ; c1ccc(-n2c3ccccc3c3cc4c(cc32)sc2ccc3c5c6ccccc6ccc5c5ccccc5c3c24)cc1]

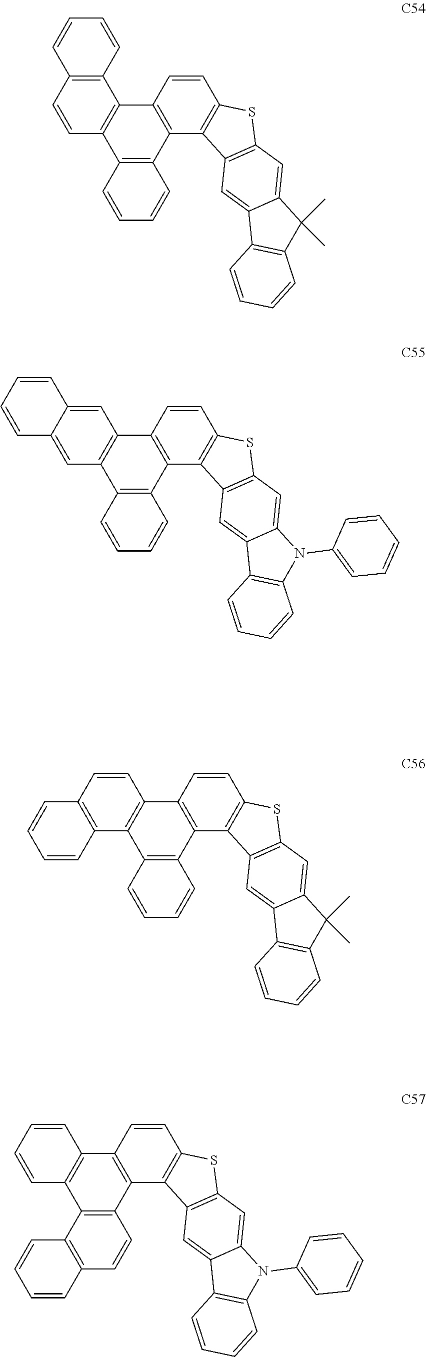 [SMILES: CC1(C)c2ccccc2-c2cc3c(cc21)sc1ccc2c4c5ccccc5ccc4c4ccccc4c2c13 ; c1ccc(-n2c3ccccc3c3cc4c(cc32)sc2ccc3c5cc6ccccc6cc5c5ccccc5c3c24)cc1 ; CC1(C)c2ccccc2-c2cc3c(cc21)sc1ccc2c4ccc5ccccc5c4c4ccccc4c2c13 ; c1ccc(-n2c3ccccc3c3cc4c(cc32)sc2ccc3c5ccccc5c5c6ccccc6ccc5c3c24)cc1]

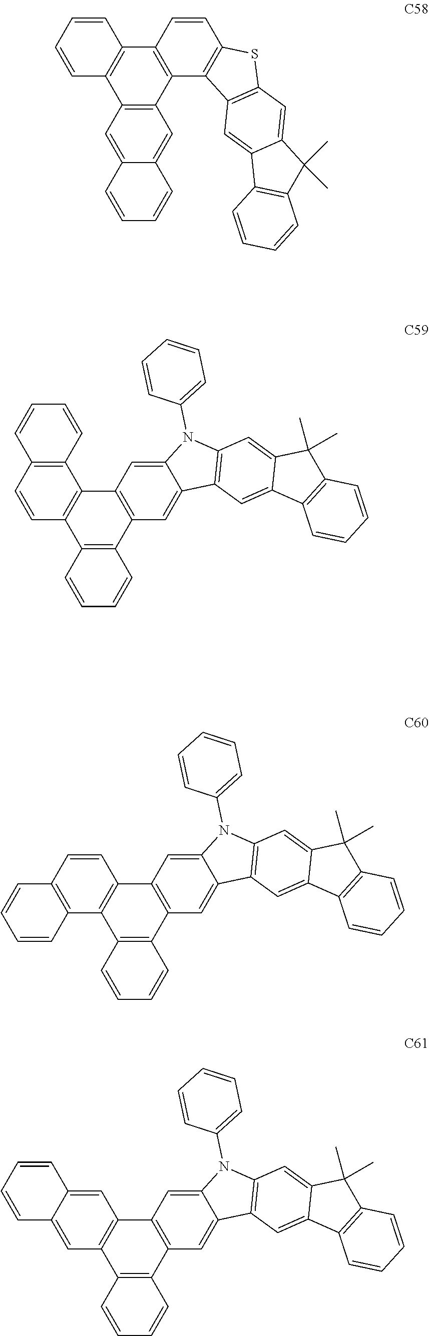 [SMILES: CC1(C)c2ccccc2-c2cc3c(cc21)sc1ccc2c4ccccc4c4cc5ccccc5cc4c2c13 ; CC1(C)c2ccccc2-c2cc3c4cc5c6ccccc6c6ccc7ccccc7c6c5cc4n(-c4ccccc4)c3cc21 ; CC1(C)c2ccccc2-c2cc3c4cc5c6ccccc6c6c7ccccc7ccc6c5cc4n(-c4ccccc4)c3cc21 ; CC1(C)c2ccccc2-c2cc3c4cc5c6ccccc6c6cc7ccccc7cc6c5cc4n(-c4ccccc4)c3cc21]

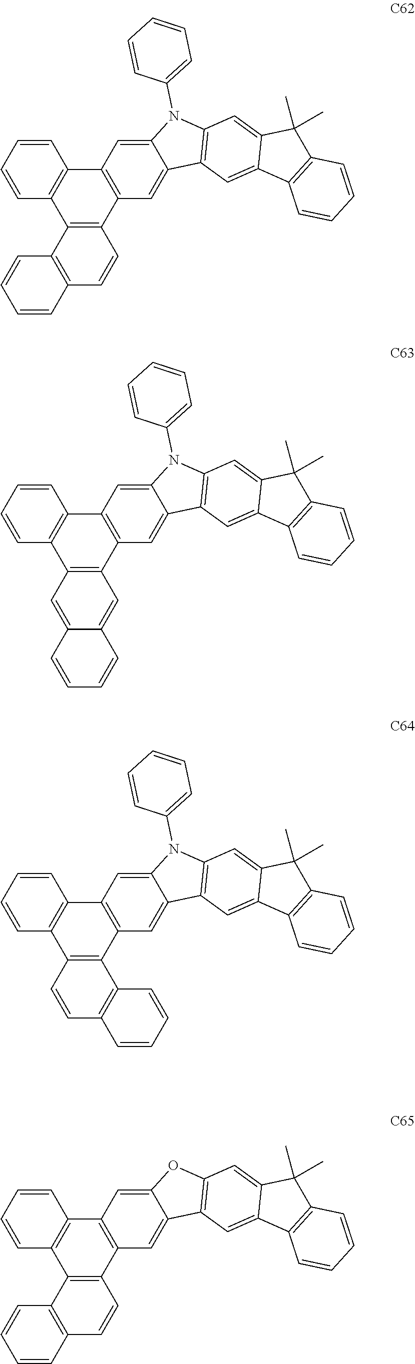 [SMILES: CC1(C)c2ccccc2-c2cc3c4cc5c(cc4n(-c4ccccc4)c3cc21)c1ccccc1c1c2ccccc2ccc51 ; CC1(C)c2ccccc2-c2cc3c4cc5c6cc7ccccc7cc6c6ccccc6c5cc4n(-c4ccccc4)c3cc21 ; CC1(C)c2ccccc2-c2cc3c4cc5c(cc4n(-c4ccccc4)c3cc21)c1ccccc1c1ccc2ccccc2c15 ; CC1(C)c2ccccc2-c2cc3c(cc21)oc1cc2c4ccccc4c4c5ccccc5ccc4c2cc13]

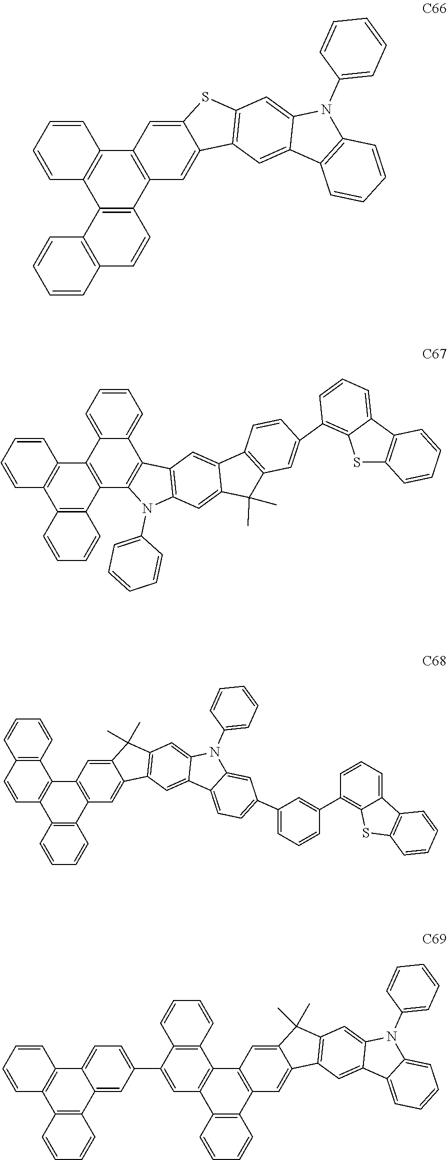 [SMILES: c1ccc(-n2c3ccccc3c3cc4c(cc32)sc2cc3c5ccccc5c5c6ccccc6ccc5c3cc24)cc1 ; CC1(C)c2cc(-c3cccc4c3sc3ccccc34)ccc2-c2cc3c4c5ccccc5c5c6ccccc6c6ccccc6c5c4n(-c4ccccc4)c3cc21 ; CC1(C)c2cc3c(cc2-c2cc4c5ccc(-c6cccc(-c7cccc8c7sc7ccccc78)c6)cc5n(-c5ccccc5)c4cc21)c1ccccc1c1ccc2ccccc2c13 ; CC1(C)c2cc3c(cc2-c2cc4c5ccccc5n(-c5ccccc5)c4cc21)c1ccccc1c1cc(-c2ccc4c5ccccc5c5ccccc5c4c2)c2ccccc2c31]

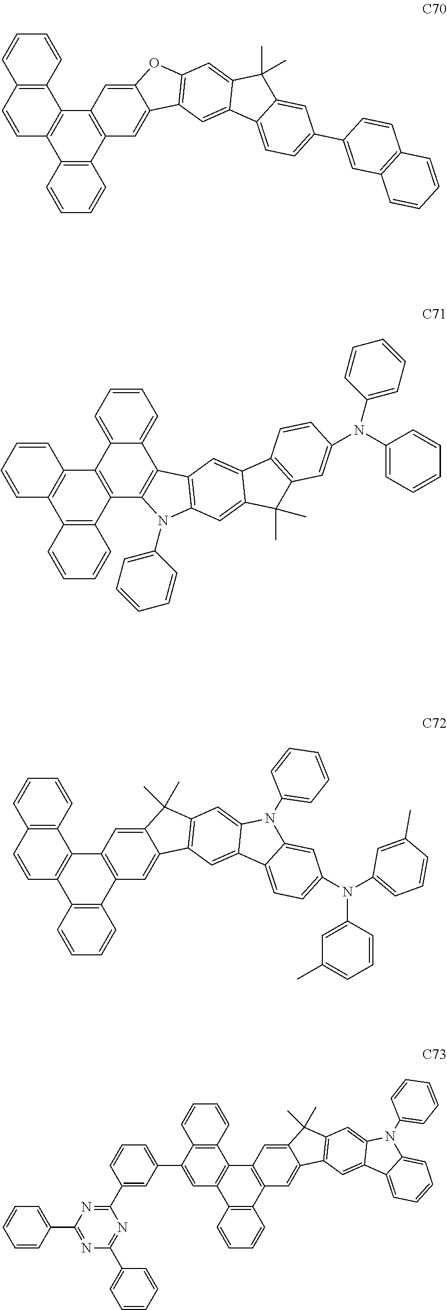 [SMILES: CC1(C)c2cc(-c3ccc4ccccc4c3)ccc2-c2cc3c(cc21)oc1cc2c(cc13)c1ccccc1c1ccc3ccccc3c12 ; CC1(C)c2cc(N(c3ccccc3)c3ccccc3)ccc2-c2cc3c4c5ccccc5c5c6ccccc6c6ccccc6c5c4n(-c4ccccc4)c3cc21 ; Cc1cccc(N(c2cccc(C)c2)c2ccc3c4cc5c(cc4n(-c4ccccc4)c3c2)C(C)(C)c2cc3c(cc2-5)c2ccccc2c2ccc4ccccc4c23)c1 ; CC1(C)c2cc3c(cc2-c2cc4c5ccccc5n(-c5ccccc5)c4cc21)c1ccccc1c1cc(-c2cccc(-c4nc(-c5ccccc5)nc(-c5ccccc5)n4)c2)c2ccccc2c31]

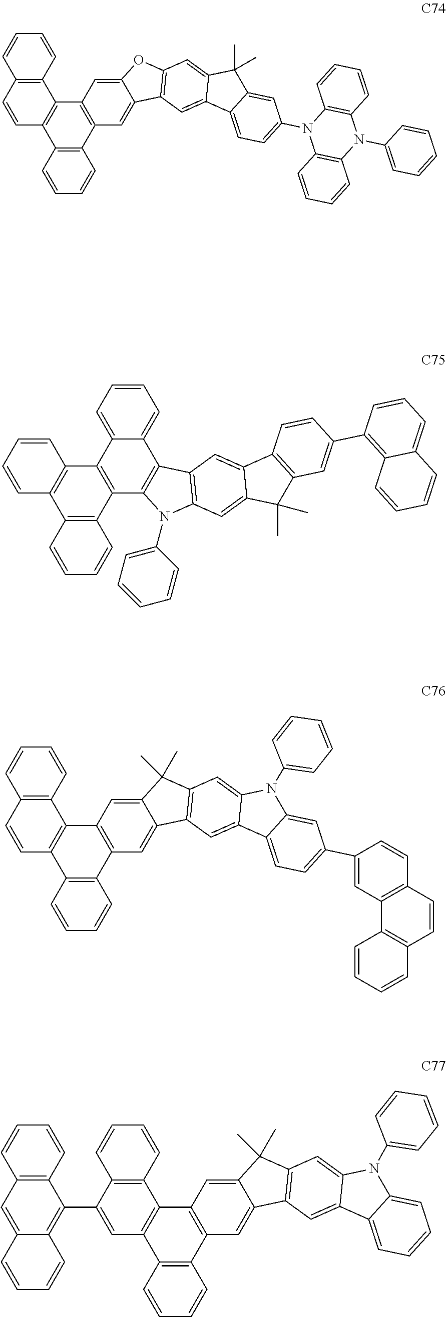 [SMILES: CC1(C)c2cc(N3c4ccccc4N(c4ccccc4)c4ccccc43)ccc2-c2cc3c(cc21)oc1cc2c(cc13)c1ccccc1c1ccc3ccccc3c12 ; CC1(C)c2cc(-c3cccc4ccccc34)ccc2-c2cc3c4c5ccccc5c5c6ccccc6c6ccccc6c5c4n(-c4ccccc4)c3cc21 ; CC1(C)c2cc3c(cc2-c2cc4c5ccc(-c6ccc7ccc8ccccc8c7c6)cc5n(-c5ccccc5)c4cc21)c1ccccc1c1ccc2ccccc2c13 ; CC1(C)c2cc3c(cc2-c2cc4c5ccccc5n(-c5ccccc5)c4cc21)c1ccccc1c1cc(-c2c4ccccc4cc4ccccc24)c2ccccc2c31]

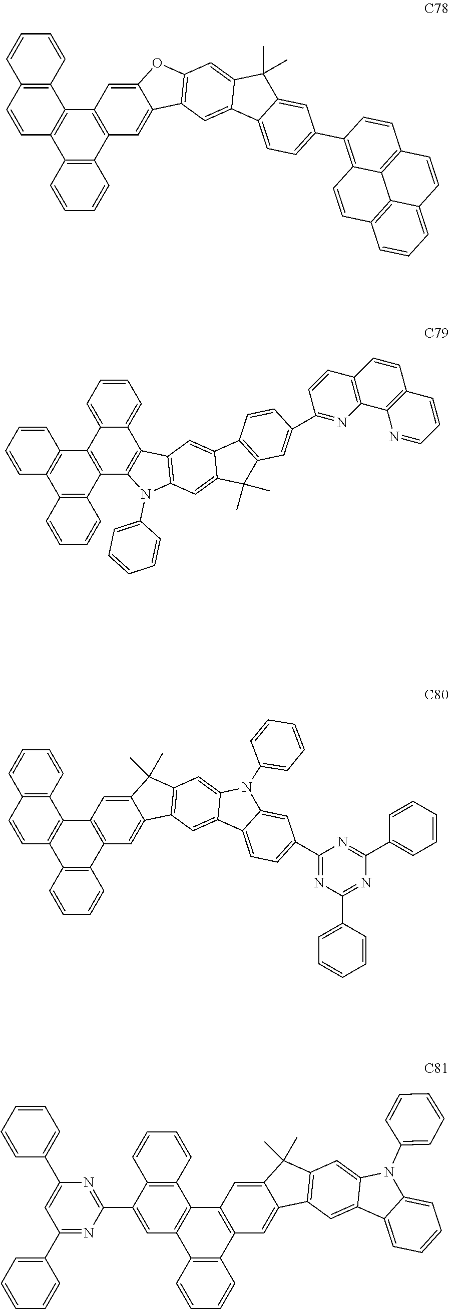 [SMILES: CC1(C)c2cc(-c3ccc4ccc5cccc6ccc3c4c56)ccc2-c2cc3c(cc21)oc1cc2c(cc13)c1ccccc1c1ccc3ccccc3c12 ; CC1(C)c2cc(-c3ccc4ccc5cccnc5c4n3)ccc2-c2cc3c4c5ccccc5c5c6ccccc6c6ccccc6c5c4n(-c4ccccc4)c3cc21 ; CC1(C)c2cc3c(cc2-c2cc4c5ccc(-c6nc(-c7ccccc7)nc(-c7ccccc7)n6)cc5n(-c5ccccc5)c4cc21)c1ccccc1c1ccc2ccccc2c13 ; CC1(C)c2cc3c(cc2-c2cc4c5ccccc5n(-c5ccccc5)c4cc21)c1ccccc1c1cc(-c2nc(-c4ccccc4)cc(-c4ccccc4)n2)c2ccccc2c31]

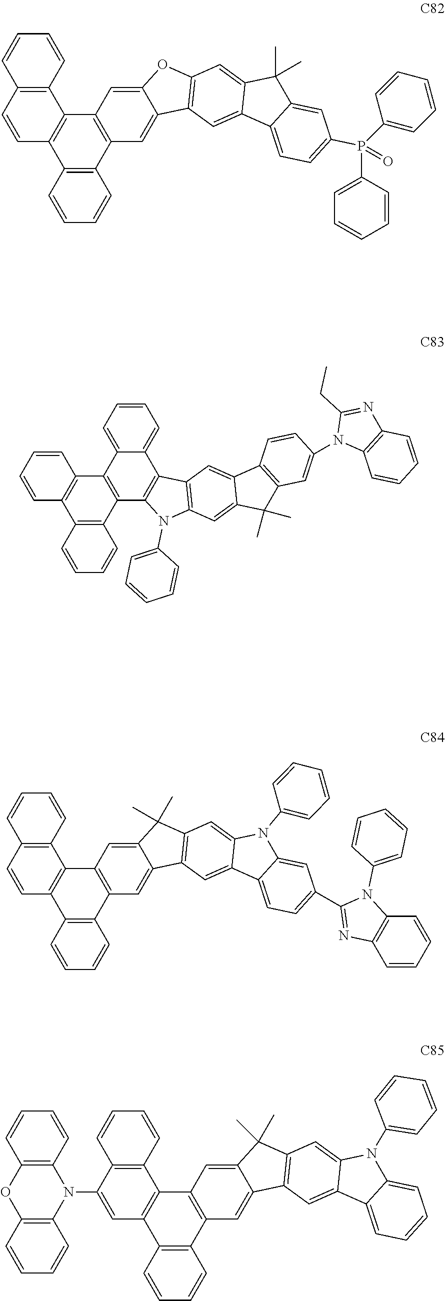 [SMILES: CC1(C)c2cc(P(=O)(c3ccccc3)c3ccccc3)ccc2-c2cc3c(cc21)oc1cc2c(cc13)c1ccccc1c1ccc3ccccc3c12 ; CCc1nc2ccccc2n1-c1ccc2c(c1)C(C)(C)c1cc3c(cc1-2)c1c2ccccc2c2c4ccccc4c4ccccc4c2c1n3-c1ccccc1 ; CC1(C)c2cc3c(cc2-c2cc4c5ccc(-c6nc7ccccc7n6-c6ccccc6)cc5n(-c5ccccc5)c4cc21)c1ccccc1c1ccc2ccccc2c13 ; CC1(C)c2cc3c(cc2-c2cc4c5ccccc5n(-c5ccccc5)c4cc21)c1ccccc1c1cc(N2c4ccccc4Oc4ccccc42)c2ccccc2c31]

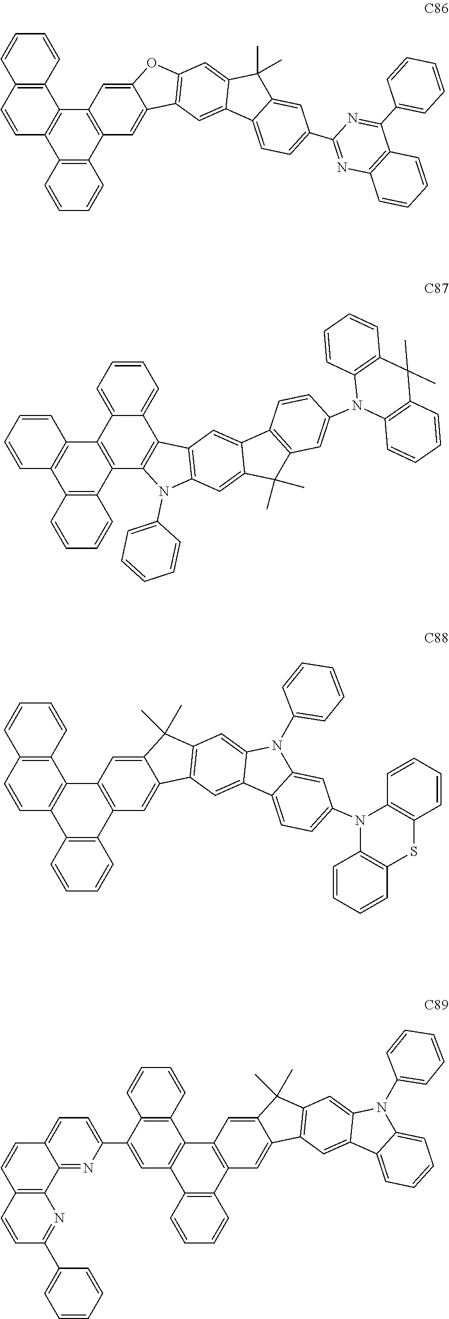 [SMILES: CC1(C)c2cc(-c3nc(-c4ccccc4)c4ccccc4n3)ccc2-c2cc3c(cc21)oc1cc2c(cc13)c1ccccc1c1ccc3ccccc3c12 ; CC1(C)c2cc(N3c4ccccc4C(C)(C)c4ccccc43)ccc2-c2cc3c4c5ccccc5c5c6ccccc6c6ccccc6c5c4n(-c4ccccc4)c3cc21 ; CC1(C)c2cc3c(cc2-c2cc4c5ccc(N6c7ccccc7Sc7ccccc76)cc5n(-c5ccccc5)c4cc21)c1ccccc1c1ccc2ccccc2c13 ; CC1(C)c2cc3c(cc2-c2cc4c5ccccc5n(-c5ccccc5)c4cc21)c1ccccc1c1cc(-c2ccc4ccc5ccc(-c6ccccc6)nc5c4n2)c2ccccc2c31]

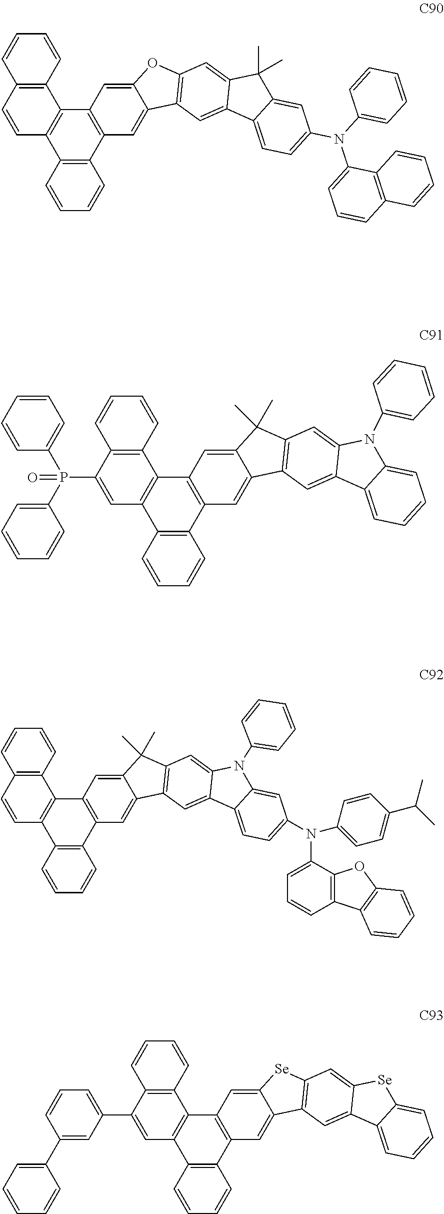 [SMILES: CC1(C)c2cc(N(c3ccccc3)c3cccc4ccccc34)ccc2-c2cc3c(cc21)oc1cc2c(cc13)c1ccccc1c1ccc3ccccc3c12 ; CC1(C)c2cc3c(cc2-c2cc4c5ccccc5n(-c5ccccc5)c4cc21)c1ccccc1c1cc(P(=O)(c2ccccc2)c2ccccc2)c2ccccc2c31 ; CC(C)c1ccc(N(c2ccc3c4cc5c(cc4n(-c4ccccc4)c3c2)C(C)(C)c2cc3c(cc2-5)c2ccccc2c2ccc4ccccc4c23)c2cccc3c2oc2ccccc23)cc1 ; c1ccc(-c2cccc(-c3cc4c5ccccc5c5cc6c(cc5c4c4ccccc34)[se]c3cc4[se]c5ccccc5c4cc36)c2)cc1]

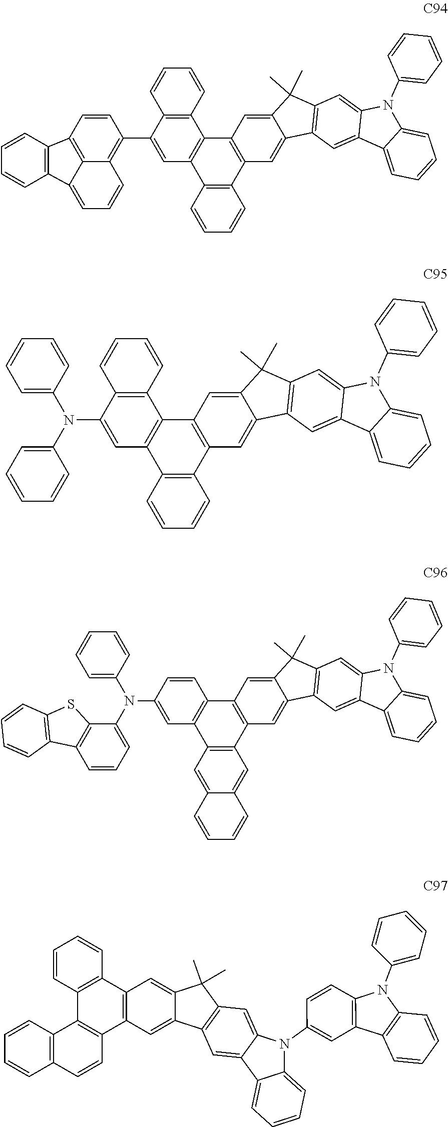 [SMILES: CC1(C)c2cc3c(cc2-c2cc4c5ccccc5n(-c5ccccc5)c4cc21)c1ccccc1c1cc(-c2ccc4c5c(cccc25)-c2ccccc2-4)c2ccccc2c31 ; CC1(C)c2cc3c(cc2-c2cc4c5ccccc5n(-c5ccccc5)c4cc21)c1ccccc1c1cc(N(c2ccccc2)c2ccccc2)c2ccccc2c31 ; CC1(C)c2cc3c4ccc(N(c5ccccc5)c5cccc6c5sc5ccccc56)cc4c4cc5ccccc5cc4c3cc2-c2cc3c4ccccc4n(-c4ccccc4)c3cc21 ; CC1(C)c2cc3c4ccccc4c4c5ccccc5ccc4c3cc2-c2cc3c4ccccc4n(-c4ccc5c(c4)c4ccccc4n5-c4ccccc4)c3cc21]

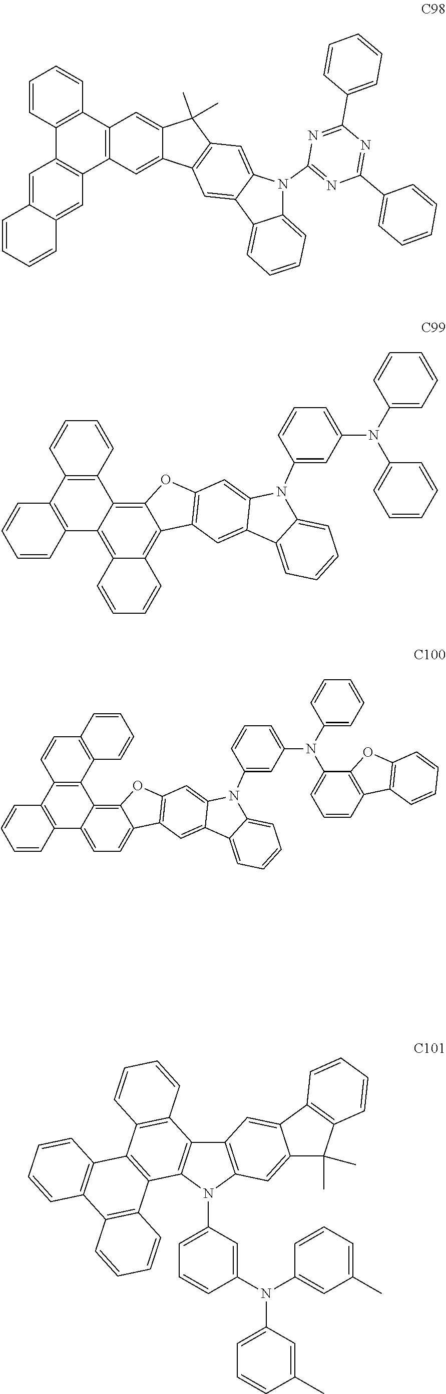 [SMILES: CC1(C)c2cc3c4ccccc4c4cc5ccccc5cc4c3cc2-c2cc3c4ccccc4n(-c4nc(-c5ccccc5)nc(-c5ccccc5)n4)c3cc21 ; c1ccc(N(c2ccccc2)c2cccc(-n3c4ccccc4c4cc5c(cc43)oc3c5c4ccccc4c4c5ccccc5c5ccccc5c34)c2)cc1 ; c1ccc(N(c2cccc(-n3c4ccccc4c4cc5c(cc43)oc3c5ccc4c5ccccc5c5ccc6ccccc6c5c43)c2)c2cccc3c2oc2ccccc23)cc1 ; Cc1cccc(N(c2cccc(C)c2)c2cccc(-n3c4cc5c(cc4c4c6ccccc6c6c7ccccc7c7ccccc7c6c43)-c3ccccc3C5(C)C)c2)c1]

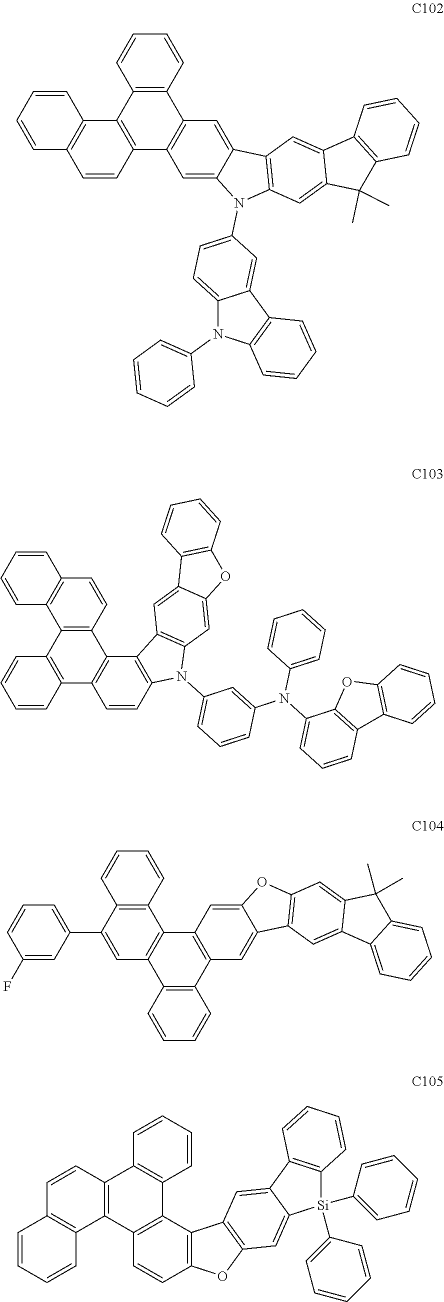 [SMILES: CC1(C)c2ccccc2-c2cc3c4cc5c6ccccc6c6c7ccccc7ccc6c5cc4n(-c4ccc5c(c4)c4ccccc4n5-c4ccccc4)c3cc21 ; c1ccc(N(c2cccc(-n3c4cc5oc6ccccc6c5cc4c4c5c(ccc43)c3ccccc3c3c4ccccc4ccc35)c2)c2cccc3c2oc2ccccc23)cc1 ; CC1(C)c2ccccc2-c2cc3c(cc21)oc1cc2c(cc13)c1ccccc1c1cc(-c3cccc(F)c3)c3ccccc3c12 ; c1ccc([Si]2(c3ccccc3)c3ccccc3-c3cc4c(cc32)oc2ccc3c5c6ccccc6ccc5c5ccccc5c3c24)cc1]

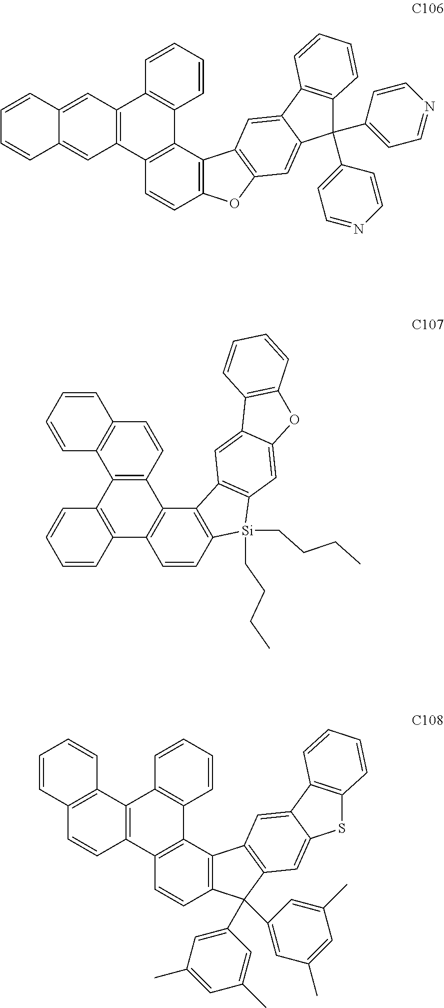 [SMILES: c1ccc2c(c1)-c1cc3c(cc1C2(c1ccncc1)c1ccncc1)oc1ccc2c4cc5ccccc5cc4c4ccccc4c2c13 ; CCCC[Si]1(CCCC)c2cc3oc4ccccc4c3cc2-c2c1ccc1c3ccccc3c3c4ccccc4ccc3c21 ; Cc1cc(C)cc(C2(c3cc(C)cc(C)c3)c3cc4sc5ccccc5c4cc3-c3c2ccc2c4ccc5ccccc5c4c4ccccc4c32)c1]

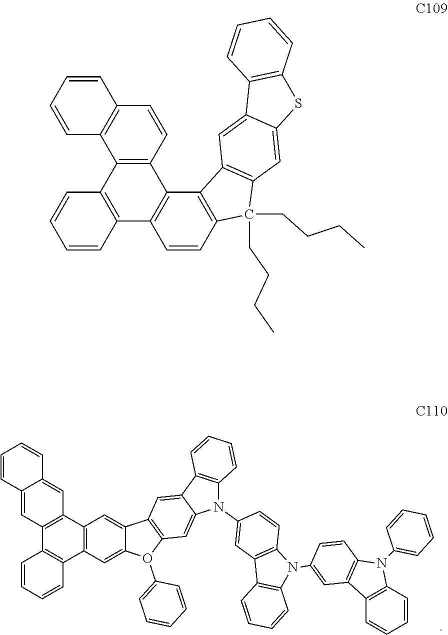

6. An organic electroluminescence device, comprising a pair of electrodes composed of a cathode and an anode, and a light emitting layer and one or more organic thin film layers between the pair of electrodes, wherein at least one of the light emitting layer and the organic thin film layer comprises the organic compound of claim 1.

7. The organic electroluminescence device of claim 6, wherein the light emitting layer comprising the organic compound of formula (1) or formula (2) is a host material.

8. The organic electroluminescence device of claim 6, wherein the light emitting layer comprising the organic compound of formula (1) or formula (2) is a fluorescent dopant material.

9. The organic electroluminescence device of claim 6, wherein the organic thin film layer comprising the organic compound of formula (1) or formula (2) is an electron transporting layer.

10. The organic electroluminescence device of claim 6, wherein the organic electroluminescence device is a lighting panel.

11. The organic electroluminescence device of claim 6, wherein the organic electroluminescence device is a backlight panel.
